SEQUENCE LISTING (64 sequences)

SEQ ID NO: 1 - DNA, 23 bp, Artificial Sequence
  Primer
cagagaccccattactggagaca

SEQ ID NO: 2 - DNA, 19 bp, Artificial Sequence
  Primer
tgacaccatcctgggcatt

SEQ ID NO: 3 - DNA, 20 bp, Artificial Sequence
  Primer
ctccggcaagtaccaattgc

SEQ ID NO: 4 - DNA, 18 bp, Artificial Sequence
  Primer
atgtgcccagggctgtgt

SEQ ID NO: 5 - DNA, 22 bp, Artificial Sequence
  Primer
cttccctcctgtcctcagaggt

SEQ ID NO: 6 - DNA, 22 bp, Artificial Sequence
  Primer
caacccagggtacaggctagtc

SEQ ID NO: 7 - DNA, 18 bp, Artificial Sequence
  Primer
ccttgtcctccccaagcg

SEQ ID NO: 8 - DNA, 22 bp, Artificial Sequence
  Primer
gccgcttcttccaaagtctaca

SEQ ID NO: 9 - DNA, 22 bp, Artificial Sequence
  Primer
catccagctcctagaagccatt

SEQ ID NO: 10 - DNA, 18 bp, Artificial Sequence
  Primer
ttgaatgggccacaggct

SEQ ID NO: 11 - DNA, 21 bp, Artificial Sequence
  Primer
gcgcagtaaagaatggcattc

SEQ ID NO: 12 - DNA, 21 bp, Artificial Sequence
  Primer
tcgattccaggtcaccacttg

SEQ ID NO: 13 - DNA, 22 bp, Artificial Sequence
  Primer
tggcaaagtggagattgttgcc

SEQ ID NO: 14 - DNA, 21 bp, Artificial Sequence
  Primer
tcgttgcacaatgacctgatc

SEQ ID NO: 15 - DNA, 25 bp, Artificial Sequence
  Primer
acaccggtagtaaatcccataaagg

SEQ ID NO: 16 - DNA, 22 bp, Artificial Sequence
  Primer
tgtccttccctttctggatgag

SEQ ID NO: 17 - DNA, 26 bp, Artificial Sequence
  Primer
aacagggtggaactgtataggaagac

SEQ ID NO: 18 - DNA, 20 bp, Artificial Sequence
  Primer
ggcgtaactaggccaggctt

SEQ ID NO: 19 - DNA, 23 bp, Artificial Sequence
  Primer
ggtggctctggacaatgtatttc

SEQ ID NO: 20 - DNA, 19 bp, Artificial Sequence
  Primer
agggcatgttgactgccat

SEQ ID NO: 21 - DNA, 22 bp, Artificial Sequence
  Primer
cgtgtctctactcccggtttcc

SEQ ID NO: 22 - DNA, 25 bp, Artificial Sequence
  Primer
gggttgcaggaacttcttaattgta

SEQ ID NO: 23 - DNA, 20 bp, Artificial Sequence
  Primer
agcggactatggaggcgtag

SEQ ID NO: 24 - DNA, 22 bp, Artificial Sequence
  Primer
ctgtcttgaggatgtccacagc

SEQ ID NO: 25 - DNA, 29 bp, Artificial Sequence
  Primer
cacaggcaataacaatatatctgaaatct

SEQ ID NO: 26 - DNA, 21 bp, Artificial Sequence
  Primer
aagatggtgatgggcttcccg

SEQ ID NO: 27 - DNA, 34 bp, Artificial Sequence
  Primer
ccttggatccatggtgatgagcccaggttctttg

SEQ ID NO: 28 - DNA, 38 bp, Artificial Sequence
  Primer
cctttctagactacggactgataaaagactcatcgaag

SEQ ID NO: 29 - DNA, 722 bp, Mus sp.
gagcttgacaccgaaggaccctgtctccaggagcacacagctagactcgtccccagttggaggaaagctggccagctttggaatcactgttggaagagatgacaatgagcccatgtcctttgttgttggtcttcgtgctgggtctggttgtgattcctccaactctggctcagaatgaaaggtacgaaaaattcctacgtcagcactatgatgccaagccaaagggccgggacgacagatactgtgaaagtatgatgaaggaaagaaagctaacctcgccttgcaaagatgtcaacacctttatccatggcaccaagaaaaacatcagggccatctgtggaaagaaaggaagcccttatggagaaaacttcagaataagcaattctcccttccagatcaccacttgtacgcactcaagagggtctccctggcctccatgcgggtaccgagcctttaaagatttcagatatattgttattgcctgtgaagatggctggcctgtccacttcgatgagtcttttatcagtccgtagacagcaggcccctggcacagacctaggtctgttttctttttatctcccctcacagccatgatcactggttcaccgttcactgtcacgggccagaaaatgaattatctgaaatatacttctcctcatttataatgcacagaaataaagatatctcaaaamccataaaaaaaaaaaaaaaaaaaaaaaaaa

SEQ ID NO: 30 - DNA, 708 bp, Mus sp.
ctctagcttcacaccgcaggaccctgtctccaggagcacgaagctagacacatcccccgttggaggaaagctggccagctttggaatctctgttggaagagatggtgatgagcccaggttctttgttgttggtctttttgctgagtctggatgtgatccctcccactctggctcaggataactacaggtacataaaattcctgactcagcactatgatgccaagccaactggccgggattacagatactgcgaaagtatgatgaagaaaagaaagctaacctcgccttgcaaagaagtcaacacctttattcatgacaccaagaacaacatcaaggccatctgtggagagaatggaaggccttatggagtaaactttagaataagcaattctcgattccaggtcaccacttgcacgcacaaaggagggtctcccaggcctccatgccagtacaatgcctttaaagatttcagatatattgttattgcctgtgaagatggctggcctgtccacttcgatgagtcttttatcagtccgtagacagcaggcccctggcacagacctaggtctgttttctttttatctcccctcacagccatgatcactggttcagcattcactgtcagtggccagaaaatgaattatctgaaatatacttctcctgatttataatgcacagaaataaagatatctcaaaaaccaaaaaaaa

SEQ ID NO: 31 - protein, 144 aa, Mus sp.
MTMSPCPLLLVFVLGLVVIPPTLAQNERYEKFLREHYDAKPKGRDDRYCESMMKERKLTSPCKDVNTFIHGTKKNIRAICGKKGSPYGENFRISNSPFQITTCTHSRGSPWPPCGYRAFKDFRYIVIACEDGWPVHFDESFISP

SEQ ID NO: 32 - protein, 145 aa, Mus sp.
MAISPGPLFLIFVLGLVVIPPTLAQDDSRYTKFLTQHHDAKPKGRDDRYCERMMKRRSLTSPCKDVNTFIHGNKSNIKAICGANGSPYRENLRMSKSPFQVTTCKHTGGSPRPPCQYRASAGFRHVVIACENGLPVHFDESFFSL

SEQ ID NO: 33 - protein, 145 aa, Mus sp.
MVMSPGSLLLVFLLSLDVIPPTLAQDNYRYIKFLTQHYDAKPTGRDYRYCESMMKKRKLTSPCKEVNTFIHDTKNNIKAICGENGRPYGVNFRISNSRFQVTTCTHKGGSPRPPCQYNAFKDFRYIVIACEDGWPVHFDESFISP

SEQ ID NO: 34 - protein, 145 aa, Mus sp.
MAMSPGPLFLVFLLGLVVIPPTLSQDDSRYTKFLTQHYDAKPKGRDDRYCESMMVKRKLTSFCKDVNTFIHDTKNNIKAICGKKGSPYGRNLRISKSRFQVTTCTHKGRSPRPPCRYRASKGFRYIIIGCENGWPVHFDESFISP

SEQ ID NO: 35 - DNA, 25 bp, Mus sp.
ctctggctcagaatgtaaggtacga

SEQ ID NO: 36 - DNA, 24 bp, Mus sp.
gaaatctttaaaggctcggtaccc

SEQ ID NO: 37 - DNA, 26 bp, Mus sp.
ctggctcaggataactacaggtacat

SEQ ID NO: 38 - DNA, 19 bp, Mus sp.
gcctgggagaccctccttt

SEQ ID NO: 39 - DNA, 20 bp, Mus sp.
agcgaatggaagcccttaca

SEQ ID NO: 40 - DNA, 20 bp, Mus sp.
ctcatcgaagtggaccggca

SEQ ID NO: 41 - DNA, 25 bp, Mus sp.
ggtgaaaagaaagctaacctctttc

SEQ ID NO: 42 - DNA, 24 bp, Mus sp.
agacttgcttattcttaaatttcg

SEQ ID NO: 43 - DNA, 24 bp, Artificial Sequence
  Primer
aagtccttggtcgggaagtataca

SEQ ID NO: 44 - DNA, 24 bp, Artificial Sequence
  Primer
actccctcaaagtcatcacaaaca

SEQ ID NO: 45 - DNA, 25 bp, Artificial Sequence
  Primer
ttaaaaacaccgagatttccttcaa

SEQ ID NO: 46 - DNA, 16 bp, Artificial Sequence
  Primer
gggccccgccatctag

SEQ ID NO: 47 - DNA, 24 bp, Artificial Sequence
  Primer
cgggacatgtttgatgactatgtc

SEQ ID NO: 48 - DNA, 25 bp, Artificial Sequence
  Primer
catcccattgaaggattcaaataaa

SEQ ID NO: 49 - DNA, 21 bp, Artificial Sequence
  Primer
caatgccaaattgctccaatt

SEQ ID NO: 50 - DNA, 16 bp, Artificial Sequence
  Primer
tggccgtgggctcagt

SEQ ID NO: 51 - DNA, 22 bp, Artificial Sequence
  Primer
tggtgaattgtctccgaaaaga

SEQ ID NO: 52 - DNA, 21 bp, Artificial Sequence
  Primer
cacgttcatcacgaggtcatg

SEQ ID NO: 53 - DNA, 21 bp, Artificial Sequence
  Primer
cctctggtgaagcccaagatc

SEQ ID NO: 54 - DNA, 19 bp, Artificial Sequence
  Primer
tctgggtttccgccagttt

SEQ ID NO: 55 - DNA, 21 bp, Artificial Sequence
  Primer
caccttcctcttcccaaagct

SEQ ID NO: 56 - DNA, 19 bp, Artificial Sequence
  Primer
gcgtcggactcggtcttct

SEQ ID NO: 57 - DNA, 23 bp, Artificial Sequence
  Primer
atgtctcacaatgccatcaggtt

SEQ ID NO: 58 - DNA, 21 bp, Artificial Sequence
  Primer
gctcgcagatcagcagactct

SEQ ID NO: 59 - DNA, 19 bp, Artificial Sequence
  Primer
gcatgccatgggcaagtac

SEQ ID NO: 60 - DNA, 24 bp, Artificial Sequence
  Primer
ccacatagatctctgccagtgttg

SEQ ID NO: 61 - DNA, 435 bp, Mus sp.
atgacaatgagcccatgtcctttgttgttggtcttcgtgctgggtctggttgtgattcctccaactctggctcagaatgaaaggtacgaaaaattcctacgtcagcactatgatgccaagccaaagggccgggacgacagatactgtgaaagtatgatgaaggaaagaaagctaacctcgccttgcaaagatgtcaacacctttatccatggcaccaagaaaaacatcagggccatctgtggaaagaaaggaagcccttatggagaaaacttcagaataagcaattctcccttccagatcaccacttgtacgcactcaagagggtctccctggcctccatgcgggtaccgagcctttaaagatttcagatatattgttattgcctgtgaagatggctggcctgtccacttcgatgagtcttttatcagtccgtag

SEQ ID NO: 62 - DNA, 438 bp, Mus sp.
atggtgatgagcccaggttctttgttgttggtctttttgctgagtctggatgtgatccctcccactctggctcaggataactacaggtacataaaattcctgactcagcactatgatgccaagccaactggccgggattacagatactgcgaaagtatgatgaagaaaagaaagctaacctcgccttgcaaagaagtcaacacctttattcatgacaccaagaacaacatcaaggccatctgtggagagaatggaaggccttatggagtaaactttagaataagcaattctcgattccaggtcaccacttgcacgcacaaaggagggtctcccaggcctccatgccagtacaatgcctttaaagatttcagatatattgttattgcctgtgaagatggctggcctgtccacttcgatgagtcttttatcagtccgtag

SEQ ID NO: 63 - DNA, 438 bp, Mus sp.
atggcgataagcccaggcccgttgttcttgatcttcgtgctgggtctggttgtgatccctcccactctggctcaggatgactccaggtacacaaaattcctgactcagcaccatgacgccaagccaaagggccgggacgacagatactgtgaacgtatgatgaagagaagaagcctaacctcaccctgcaaagatgtcaacacctttatccatggcaacaagagcaacatcaaggccatctgtggagcgaatggaagcccttacagagaaaacttaagaatgagcaagtctcccttccaggtcaccacttgcaagcacacaggagggtctccccggcctccatgccagtaccgagcctctgcagggttcagacatgttgttattgcctgtgagaatggcttgccggtccacttcgatgagtcatttttcagtctatag

SEQ ID NO: 64 - DNA, 438 bp, Mus sp.
atggcgatgagcccaggtcctttgttcttggtcttcctgttgggtctggttgtgatccctcccactctgtctcaggatgactccaggtacacaaaattcctgactcagcactatgatgccaagccaaaaggccgggacgacagatactgcgaaagtatgatggtgaaaagaaagctaacctctttctgcaaagatgtcaacacctttatccatgacaccaagaacaacatcaaggccatctgtggaaagaaaggaagcccttatggacgaaatttaagaataagcaagtctcgcttccaggtcaccacttgcacacacaaaggaaggtctccccggcctccatgcaggtaccgagcctctaaagggttcagatatattattattggctgtgagaatggctggcctgtccactttgatgagtcttttatcagtccatag

(19) United States
(12) Patent Application Publication
(10) Pub. No.: US 2005/0239706 A1
(43) Pub. Date: Oct. 27, 2005

(54) Modulation of fiaf and the gastrointestinal microbiota as a means to control energy storage in a subject

(75) Inventors: Fredrik Backhed, St. Louis, MO (US); John Rawls, St. Louis, MO (US); Justin Sonnenburg, St. Louis, MO (US); Lora V. Hooper, Coppell, TX (US); Jeffrey I. Gordon, St. Louis, MO (US)

Correspondence Address: POLSINELLI SHALTON WELTE SUELTHAUS P.C., 700 W. 47TH STREET, SUITE 1000, KANSAS CITY, MO, 64112-1802, US

(73) Assignee: Washington University in St. Louis

(21) Appl. No.: 11/080,755

(22) Filed: Mar. 15, 2005

Related U.S. Application Data
(63) Continuation-in-part of application No. 10/432,819, filed on Oct. 31, 2003.
(60) Provisional application No. 60/591,313, filed on Jul. 27, 2004.

(51) Int. Cl. A61K 38/17

(52) U.S. Cl. 514/12

(57) ABSTRACT

The invention provides compositions and methods to modulate fat storage and weight loss in a subject. In certain aspects of the invention, fat storage (adiposity) and weight loss is modulated by altering the subject's gastrointestinal microbiota population. In other aspects of the invention, fat storage and weight loss is modulated by altering the amount of or the activity of the protein, fasting-induced adipocyte factor, in the subject.

### CROSS-REFERENCE TO RELATED APPLICATIONS

This application claims priority from Provisional Application Ser. No. 60/591,313 filed on Jul. 27, 2004, and is a continuation-in-part application of application Ser. No. 10/432,819 filed on Nov. 27, 2001, which claims priority from Provisional Application Ser. No. 60/252,901 filed on Nov. 27, 2000, all of which are hereby incorporated by reference in their entirety.

### FIELD OF THE INVENTION

The current invention generally relates to the effects of the gastrointestinal microbiota on the regulation of energy storage in a subject. In particular, the invention provides compositions and methods to modulate fat storage in a subject by increasing either the amount of or the activity of the fasting-induced adipose factor protein in the subject

### BACKGROUND OF THE INVENTION

According to the Center for Disease Control (CDC), over sixty percent of the United States population is overweight, and almost twenty percent are obese. This translates into 38.8 million adults in the United States with a Body Mass Index (BMI) of 30 or above. Obesity is also a world-wide health problem with an estimated 500 million overweight adult humans [body mass index (BMI) of 25.0-29.9 kg/m²] and 250 million obese adults (Bouchard, C (2000) N Engl J Med. 343, 1888-9). This epidemic of obesity is leading to worldwide increases in the prevalence of obesity-related disorders, such as diabetes, hypertension, as well as cardiac pathology, and non-alcoholic fatty liver disease (NAFLD; Wanless, and Lentz (1990) Hepatology 12, 1106-1110. Silverman, et al, (1990). Am. J Gastroenterol. 85, 1349-1355; Neuschwander-Tetri and, Caldwell (2003) Hepatology 37, 1202-1219).

According to the National Institute of Diabetes, Digestive and Kidney Diseases (NIDDK) approximately 280,000 deaths annually are directly related to obesity. The NIDDK further estimated that the direct cost of healthcare in the U.S. associated with obesity is $51 billion. In addition, Americans spend $33 billion per year on weight loss products. In spite of this economic cost and consumer commitment, the prevalence of obesity continues to rise at alarming rates. From 1991 to 2000, obesity in the U.S. grew by 61%.

Although the physiologic mechanisms that support development of obesity are complex, the medical consensus is that the root cause relates to an excess intake of calories compared to caloric expenditure. While the treatment seems quite intuitive, dieting is not an adequate long-term solution for most people; about 90 to 95 percent of persons who lose weight subsequently regain it. Although surgical intervention has had some measured success, the various types of surgeries have relatively high rates of morbidity and mortality.

Pharmacotherapeutic principles are limited. In addition, because of undesirable side effects, the FDA has had to recall several obesity drugs from the market. Those that are approved also have side-effects. Currently, two FDA-approved anti-obesity drugs are orlistat, a lipase inhibitor, and sibutramine, a serotonin reuptake inhibitor. Orlistat acts by blocking the absorption of fat into the body. An unpleasant side effect with orlistat, however, is the passage of undigested oily fat from the body. Sibutramine is an appetite suppressant that acts by altering brain levels of serotonin. In the process, it also causes elevation of blood pressure and an increase in heart rate. Other appetite suppressants, such as amphetamine derivatives, are highly addictive and have the potential for abuse. Moreover, different subjects respond differently and unpredictably to weight-loss medications.

In summary, current surgical and pharmacotherapy treatments are problematic. Novel non-cognitive strategies are needed to prevent and treat obesity and obesity-related disorders.

### SUMMARY OF THE INVENTION

The applicants have discovered novel treatment strategies that may be employed to treat obesity and to promote weight loss. Briefly, the present discovery was made by studying the impact of the gastrointestinal microbiota on energy storage in a subject. The human gut contains an immense number of microorganisms, collectively known as the microbiota. There are approximately 500 to 1000 species of microorganisms whose collective genomes (the “microbiome”) are estimated to contain more than 100 times more genes than the human genome. The microbiota is a metabolic organ that performs functions humans cannot. These finctions, for example, include the ability to process otherwise indigestible components of the human diet, such as plant polysaccharides.

By studying the impact of the microbiota on a subject's energy balance, the applicants have discovered that the microbiota acts through an integrated host-signaling pathway to regulate energy storage in the subject. In particular, the applicants have discovered that the microbiota suppresses a subject's transcription of Fiaf in the gastrointestinal tract. Moreover, the applicants have shown that microbial-mediated suppression of Fiaf causes a subject to store body fat. While Fiaf has previously been shown to inhibit lipoprotein lipase (LPL) in vitro, a direct in vivo causal connection between Fiaf's role in the regulation of energy storage in a subject has not been previously demonstrated. In particular, the role played by the gastrointestinal microbiota in this process has not been previously demonstrated.

Among the several aspects of the current invention, therefore, is the provision of compositions and methods that may be utilized to regulate energy storage in a subject. In certain aspects of the invention, fat storage and weight loss are modulated by altering the structure or finction of the subject's gastrointestinal microbiota, or by administering chemical entities that regulate (host) intestinal Fiaf expression.

Other aspects and embodiments of the invention are described in more detail herein.

### BRIEF DESCRIPTION OF THE FIGURES

FIG. 1 shows the results of real-time quantitative RT-PCR studies of colonization-associated changes in gene expression in laser capture microdissected (LMC) ileal cell populations of a colonized mouse. Also shown is the process of LCM of the ileum in a colonized mouse. Sections were stained with nuclear fast red. Bars=25 μm.

FIG. 2 shows the results of real-time quantitative qRT-PCR analyses of mRNA levels in isolated from laser-captured cell populations. Values are expressed relative to levels in germ-free mesenchyme using ΔΔCT analysis described below. Each gene product per sample was assayed in triplicate in 3-4 independent experiments. Representative results (mean +/−1 S.D.) from pairs of germ-free and colonized mice are plotted.

FIG. 3 shows the results of an experiment to illustrate the specificity of host responses to colonization with different members of the microbiota. Germ-free mice were inoculated with one of the indicated organisms, or with a complete ileal/cecal microbiota from conventionally raised mice (CONV-R microbiota) (J. M. Friedman, Nat Med 10, 563-9 (2004)). Ileal RNAs, prepared from animals colonized at 107 CFU/ml ileal contents 10 days after inoculation, were pooled, and levels of each mRNA shown were analyzed by real time quantitative RT-PCR (qRT-PCR). Mean values (mean +/−1 S.D.) for triplicate determinations are plotted.

FIG. 4 shows the nucleotide sequences of mouse angiogenin-4 and angiogenin-3 in alignment (SEQ ID NOS 29 and 30 respectively).

FIG. 5 illustrates the sequence alignment of the amino acid sequences of mouse angiogenin family members (SEQ ID NOS 31-34).

FIG. 6 shows the locations of primers specific for mouse angiogenin family members.

FIG. 7 is a graph illustrating tissue distribution of angiogenin-4 mRNA, together with the results of an agarose gel analysis.

FIG. 8 is a graph illustrating tissue distribution of angiogenin-1 mRNA.

FIG. 9 is a graph illustrating tissue distribution of angiogenin-3 mRNA following quantitative real-time RT-PCR analysis.

FIG. 10 shows the results of RT-PCR analysis showing the absence of angiogenin-related protein gene expression.

FIG. 11 is a set of graphs showing the results of experiments on the microbial regulation of angiogenin-4 expression in the small intestine.

FIG. 12 is a graph showing the regulation of angiogenin-4 expression during postnatal development.

FIG. 13 is a graph showing cellular localization of angiogenin-4 expression in small intestine: qRT-PCR analysis of cells isolated from the crypt base.

FIG. 14 depicts a series of graphs detailing phenotype characteristics of wild-type gnotobiotic mice. Three groups of 8 week-old adult male C57B1/6J mice (abbreviated B6)- those raised in a germ-free state (GF), those allowed to acquire a microbiota from birth to adulthood (conventionally-raised; CONV-R) and those raised GF until adulthood and then colonized for 2 weeks with an unfractionated cecal microbiota harvested from CONV-R donors (conventionalized; CONV-D) were analyzed for:
14(A) total body fat content by DEXA (n=21-25/group);
14(B) epididymal fat weight (n=10-20/group);
14(C) chow consumption (average daily value over the 3 day period prior to termination of the experiment; n=10/group); and
14(D) oxygen consumption (VO₂; defined by open circuit calorimetry just prior to sacrifice; n=10/group). Mean values±SEM are plotted.

FIG. 15 depicts a series of graphs detailing the impact of a 14-day conventionalization of wild-type GF B6 mice. Sera were obtained after a 4-hour fast and analyzed for:
15(A) leptin, insulin, and glucose (n=8 animals/group). Numbers represent mean values±SEM.
15(B, C) Glucose- and insulin-tolerance tests were performed after a 4 hour fast (n=8 mice/group). Mean values i SEM are plotted

FIG. 16 depicts a series of graphs and images detailing the impact of conventionalization on hepatic lipogenesis and nuclear import of the bHLH transcription factor, ChREBP.
16(A) Oil-red O stains of paraformaldehyde-fixed liver sections prepared from 8 week-old, wild-type, male GF and CONV-D B6 mice.
16(B) Liver triglyceride levels.
16(C) qRT-PCR assays of livers from GF and CONV-D mice [n=15/group; mean values±SEM are expressed relative to levels in GF animals (GF set at 100%)].
16(D) Immunohistochemical study of paraformaldehyde-fixed sections of liver from GF or CONV-D mice. Sections were stained with rabbit polyclonal antibodies to mouse CHREBP (green). Nuclei are labeled dark blue with 4′,6-diamidino-2-phenylindole. Bars, 25 μm.

FIG. 17 depicts a series of graphs and images detailing the impact of conventionalization on adipocyte hypertrophy and Fiaf expression in the intestine.
17(A) Epididymal fat pads (left half of the panel) from 8 week-old wild-type male GF, CONV-D, and CONV-R B6 mice. The corresponding hematoxylin- and eosin-stained sections are shown in the right half of the panel.
17(B) qRT-PCR assays of epididymal fat pad RNAs harvested from wild-type mice reveal that conventionalization does not produce significant changes in expression of mediators or biomarkers of lipogenesis and adipogenesis in white fat tissue.
17(C) LPL activity is increased upon colonization in both epididymal fat pads and heart.
17(D) qRT-PCR assays of Fiafexpression in wild-type animals.
17(E) Generation of Fiafknockout mice. Structures are shown for the wild-type Fiaf locus, the targeting vector, and the mutated locus with exons 1-3 replaced by a βgeopA cassette. The desired disruption was verified by Southern blot analysis. Northern blots of adipocyte RNA establish the absence of detectable Fiaf mRNA in Fiaf−/−animals.
17(F) The absence of Fiaf markedly attenuates the increase in total body fat content following a 14 day conventionalization.

FIG. 18 is a diagram illustrating the impact of the gastrointestinal microbiota on a subject's energy storage.

FIG. 19 is graph depicting the distribution of the 10 most abundant microbial genera in the cecal microbiota of conventionalized B6 mice.

FIG. 20 is a graph depicting developmental regulation of Fiaf expression in the small intestine of germn-free (GF) and conventionally-raised (CONV-R) mice.

FIG. 21 depicts transcription factor binding sites conserved in orthologous mouse, rat, human, zebrafish and fugu Fiaf genes.
21 (A) depicts two motifs that are predicted by PhyloCon, together with the closest matches in the TRANSFAC database;
21(B) depicts selected TRANSFAC motifs, including fork head boxes, E-boxes and inferon responsive elements.

FIG. 22 depicts a series of graphs detailing the impact of a 14 day conventionalization on Ppara +/+ and Ppara −/— littermates.
22(A) is a graph depicting the expression levels of the transcription factor Ppar-α that were examined by qRT-PCR in various tissues from GF and conventionalized CONV-D CB57/B6J animals;
22(B) is a graph depicting DEXA measurements of total body fat content in Ppara +/+ and Ppara −/− mice (n=8/group); and
22(C) is a graph depicting qRT-PCR assays of Fiaf expression in Ppara +/+ and Ppara −/− mice (n=8/group). Values in panels A and C are expressed as percentages of GF (mean±SEM).

FIG. 23 depicts a series of graphs showing that zebrafish ortholog of mouse and human Fiaf/Angptl4 is suppressed by a soluble microbial factor.
23(A) is a graph showing the phylogenetic comparison of Angptl4/Fiaf and Angpt13 protein sequences in Zebrafish (Danio rerio), Fugu (Fugu rubripes), Mouse (Mus musculus), and Human (Homo sapiens). The closely related Angptl4/Fiaf and Angpt13 protein families are shown with Human ANGPTL1 used as a root (all other Angiopoietin-like and Angiopoietin proteins cluster with ANGPTL1; data not shown). Sequences were aligned with ClustalW using the BLOSUM matrix, then a parsimony tree was constructed. Numbers at each branch point indicate the subset of 1000 bootstrap replicates of heuristic searches in which this topology was supported. Branch points with bootstrap support of >700 out of 1000 are considered statistically robust. The zebrafish Fiaf ortholog is indicated by an asterisk.
23(B) is a graph showing the impact of colonization of 3 dpf germ-free zebrafish with a microbiota harvested from conventionally-raised zebrafish (CONV), or with A. hydrophila (A. h.), P. aeruginosa (P. a.), or E. coli (E. c.). The downregulation of Fiaf in the digestive tracts of colonized 6 dpf compared to GF controls shows microbial specificity.
23(C) is a graph depicting the effects of fasting on Fiaf expression. GF (black bars) and CONV-D (white bars) zebrafish were either fed beginning on 3 dpf (fed) or not fed (fasted). Fiaf mRNA levels in their digestive tracts assessed on 6 dpf.
23(D) is a graph depicting the effect of mono-association with E. coli causes mono-associated downregulation of Fiaf compared to GF the same result occurs when GF fish are separated from live E. coli by a 0.4 μm membrane, or are inoculated with heat-killed E. coli.

In panels B and D, the Y-axis indicates FiafmRNA fold-change relative to a GF baseline (note inverted scale). In panel C, the Y-axis indicates percent Fiaf mRNA levels relative to fed GF larvae. Quantitative RT-PCR assays of digestive tract RNA in panels B-D were performed in triplicate with biological duplicate pools (5-10 animals/pool) for each treatment, and normalized to 18S rRNA levels. Error bars indicate standard error of the mean.

FIG. 24 depicts a series of photographic images detailing the results of morphologic studies of CONV-R, CONV-D, and GF zebrafish.

24(A-C) are photographic images of whole-mount preparations of 6 dpf zebrafish. Rostral is to the left, dorsal is to the top. Panel (A) shows the position of the swim bladder (SB) and the boundary of intestinal segment 2 (red bracket). Segments 1 and 3 lie rostral and caudal to segment 2, respectively.

24(D-F) are photographic images of whole mounts of the caudal regions of 9 dpf CONV-R, GF, and CONV-D (conventionalized at 3 dpf) animals, showing onset of epidermal degeneration phenotype in GF fish. This phenotype is manifested by loss of transparency and integrity of the epidermis in fin folds (the edges of these fin folds are highlighted with open arrowheads in E). CONV-R and CONV-D fin folds remain transparent (edges indicated by filled black arrowheads in D and F).

24(G, H, J and K) are photographic images depicting hematoxylin- and eosin-stained transverse sections showing intestinal segment 1 (G and J) and segment 2 (H and K) in 6-dpf CONV and GF zebrafish. There are no detectable epithelial abnormalities in intestinal segment 1, whether judged by light microscopy (G and J) or by transmission EM (data not shown). In contrast, enterocytes in segment 2 contain prominent supranuclear vacuoles filled with eosinophilic material in CONV-D (and CONV-R) fish (e.g., black arrowheads in H). These vacuoles appear clear in GF animals (e.g., open arrowheads in K). Pigmented melanocytes (m) lie adjacent to the intestine in Hand K.

24(I and L) are photographic images depicting EM study of 6-dpf intestines, showing electron-dense material in the supranuclear vacuoles (v) of segment 2 CONV-D enterocytes, and electron-lucent material in GF enterocytes. The filled black arrowhead in I points to a bacterium in the intestinal lumen. (Bars: 500 μm in A-F; 100 μm in G and J; 20 μm in H and K; 5 μm in I and L.).

FIG. 25 depicts a series of photographic and graph images detailing microbiota-stimulated intestinal epithelial proliferation in zebrafish.

25(A and B) are photographic images showing sections prepared from the intestines of 6-dpf CONV-D and GF zebrafish after a 24-h exposure to bromodeoxyuridine in their environmental water. Sections were incubated with antibodies to bromodeoxyuridine (magenta) and the nuclear stain bisbenzimide (blue). The mesenchyme and muscle surrounding the intestinal epithelium are outlined in white.

25(C) is a graphic quantitation of S-phase cells in the intestinal epithelium and mesenchyme. The percentage of cells in S phase in GF intestinal epithelium is significantly lower than in CONV-R or CONV-D animals (P<0.0001, indicated by brackets with three asterisks). Data are expressed as the mean of two independent experiments±SEM (n=19-31 sections scored per animal; >6 animals per experiment). Bars, 25 μm in A and B.

FIG. 26 is a series of graphs showing real-time quantitative RT-PCR studies of the microbial species specificity of selected evolutionarily conserved zebrafish responses to the digestive tract microbiota. Expression levels of serum amyloid A1 (Saal; A), complement component 3 (C3; B), fasting-induced adipose factor (Fiaf, C), and solute carrier family 31 member 1 (Slc3lal; D) in digestive tracts from 6-dpf conventionalized (CONV-D), A. hydrophila-monoassociated (A.h.), and P. aeruginosa-monoassociated (P.a.) larvae are shown relative to 6-dpf GF larval digestive tracts. Assays were performed in triplicate (n>4 assays per gene). Data were normalized to 18S ribosomal RNA and results are expressed as mean log₂ values±SEM.

FIG. 27 depicts a series of photographic images showing the distribution of B. thetaiotaomicron within its intestinal niche.

27(A) is a low power view of the distal small intestine of B. thetaiotaomicron mono-associated gnotobiotic mouse showing a villus (arrow) viewed from above.

27(B-D) depicts progressively higher power views showing B. thetaiotaomicron associated with luminal contents (food particles, shed mucus) (arrows), and embedded in the mucus layer overlying the epithelium (boxed region in C, and panel D). Bars: A, 50 μm; B, C, 5 μm; D, 0.5 μm.

FIG. 28 depicts a series of graphs showing carbohydrate foraging by B. thetaiotaomicron.

28(A) B. thetaiotaomicron gene expression during growth from log to stationary phase in minimal medium containing 0.5% glucose or 0.5% maltotriose (a simplified starch composed of three a 1-4 linked glucose residues) versus the ceca of mono-associated gnotobiotic mice fed a polysaccharide-rich diet. Predicted operons are shown together with their component gene products. All genes listed were significantly upregulated in vivo relative to MM-G. Note that during growth in MM-G versus MM-M only 13 of the 4719 genes queried exhibit a ≧10-fold difference in their expression. Eight of these genes comprise a starch utilization system (Sus) operon: its three Sus alpha-amylases are the only ones among 241 B. thetaiotaomicron glycoside hydrolases and polysaccharide lyases whose expression change ≧10-fold as a result of exposure to maltotriose, underscoring the specificity of the organism's induced responses to the glycosidic linkages that it must process (e.g., compare alpha- and beta-glucosidases in panel B plus data in panel C).

28(B, C) Selective induction of glycoside hydrolases in vivo. Panel B, induction of expression of groups of glycoside hydrolases in the cecum compared to MM-G and MM-M (see Table S4 for a list of genes; the number of genes in each group is indicated in parenthesis; summed GeneChip signals for B. thetaiotaomicron transcripts called “Present” for individual samples within an experimental group were averaged to calculate the aggregate mean signal±S.E.M.). (C) Biochemical evidence of B. thetaiotaomicron's “preparedness” for degrading glycans. Lysates were generated from bacteria during late-log phase growth in MM-G. The organism produces a portfolio of hydrolases capable of processing a wide variety of glycosides, even when exposed to a single fermentable monosaccharide. Mean values±S.D. of triplicate assays are plotted.

28(D) GC-MS of neutral and amino sugars in cecal contents from germ-free versus B. thetaiotaomicron-colonized mice.

FIG. 29 depicts a schematic showing diet-associated changes in the in vivo expression of B. thetaiotaomicron glycoside hydrolases and polysaccharide lyases. Unsupervised hierarchical clustering yields the following groups of genes upregulated an average of >2.5-fold in vivo compared to their average level of expression at all growth phases in MM-G: Group 1, highest expression on a simple sugar diet, includes activities required for degradation of host glycans; Group 2, equivalent expression on both diets; Group 3, highest on a polysaccharide-rich standard chow diet; includes enzymes that degrade plant glycans.

FIG. 30 depicts a graph showing growth of B. thetaiotaomicron in a chemostat under various nutrient conditions. Curves show the average OD600 of duplicate B. thetaiotaomicron cultures during growth in minimal medium plus 0.5% glucose (MM-G), minimum medium plus 0.5% maltotriose (MM-M), or a control rich medium (TYG; 1% tryptone, 0.5% yeast extract, 0.2% glucose). Bacteria were harvested at the time points noted by open symbols.

FIG. 31 is a schematic showing the hierarchical clustering of B. thetaiotaomicron transcriptional profiles in vitro and in vivo.

31(A) The quality of replicates was assessed using unsupervised clustering (centroid linkage method) of samples using 4014 of 4823 probe sets that were (i) called “Present” by dChip and (ii) had signal values ≧100 in at least 1 of 16 samples. MM-G samples represent the time points shown in FIG. 30 (A and B refer to samples taken from independent vessels in the chemostat). Each of the in vivo samples was prepared from the cecal contents of a gnotobiotic mouse after a 10 day colonization (numbers refer to individual animals, all of which were maintained on a high polysaccharide standard chow diet).

31(B) Unsupervised clustering (centroid linkage method) using expression values of 98 B. thetaiotaomicron genes from the “replication, recombination and repair” COG that satisfy the same criteria used in panel A above. The 42 B. thetaiotaomicron samples consist of 12 cecal populations [nine from mice fed a polysaccharide-rich standard chow (purple), three from mice fed a simple sugar diet (ochre)], plus five time points during growth in MM-G, MM-M, or TYG (each time point assayed in duplicate cultures, designated A and B). The results reveal that all of the cecal bacterial populations cluster most closely to log phase cells irrespective of diet.

FIG. 32 depicts schematics showing COG categorization of B. thetaiotaomicron genes with increased expression in the cecum.

32(A) Genes exhibiting significantly different expression during growth in the cecum of mice fed a standard polysaccharide-rich chow diet compared to growth ex vivo in MM-G. Three groups of genes with assignable COGs are considered: 442 of the 1237 (36%) genes showing higher expression in vivo (designed as Up and shown in blue); 278 of 519 (54%) genes showing lower expression in vivo (Down; yellow) and 1845 of the 4779 genes in the genome (green). The x-axis plots the percentage of each group that falls within a given COG. Note that the largest group of genes upregulated in vivo belongs to the “carbohydrate transport and metabolism” COG, while the largest group of genes downregulated in vivo are members of the “amino acid transport and metabolism” COG.

32(B) COG comparisons of genes upregulated in the ceca of mice fed a standard polysaccharide-rich chow or high sugar diet compared to MM-G. The largest group of genes upregulated in all three in vivo experiments belong to the “carbohydrate transport and metabolism” COG.

FIG. 33 depicts a schematic showing components of B. thetaiotaomicron's polysaccharide acquisition and degradation machinery upregulated in the ceca of gnotobiotic mice fed a standard polysaccharide-rich chow diet. B. thetaiotaomicron contains 106 SusC paralogs postulated to be conserved components of a series of multifunctional outer membrane porins, and 57 SusD paralogs thought to function as specificity elements. Thirty-seven SusC and 16 SusD homologs exhibited >10-fold higher levels of expression in the cecum compared to MM-G (range 11-to 2523-fold; panel A). Each induced SusD gene is physically linked to a SusC paralog in the B. thetaiotaomicron genome: 13 adjacent pairs of upregulated SusC-SusD paralogs are members of predicted operons. Thirty-seven glycoside hydrolases and polysaccharide lyases were upregulated ≧10-fold in vivo (Panel B). Fold differences in average level of expression in vivo compared to all phases of growth in MM-G are indicated.

FIG. 34 depicts schematics detailing an example of B. thetaiotaomicron expression data placed on KEGG metabolic pathways.

34(A) “Pentose and Glucuronate Interconversions” KEGG map showing average fold difference in expression of B. thetaiotaomicron genes in the mouse cecum compared to growth in MM-G.

34(B) Higher power view of boxed region in panel A, highlighting in vivo upregulation of genes encoding putative enzymes required for metabolism of arabinose and xylose (solid arrows) to intermediates that enter the pentose phosphate pathway (open arrow).

FIG. 35 depicts a schematic detailing diet-associated changes in the in vivo expression of B. thetaiotaomicron SusC/D paralogs. Unsupervised hierarchical clustering yields two distinct groups of genes upregulated an average of ≧2.5-fold in vivo compared to their average level of expression at all growth phases in MM-G: Group 1, highest expression on a simple sugar diet; Group 2, highest expression on a polysaccharide-rich standard chow diet. An average fold difference in expression is given for each gene in each of the two groups (defined by white boxes) relative to MM-G.

FIG. 36 depicts a schematic showing diet-regulated operons. Candidate SusC/D paralogs were checked for proximity in the B. thetaiotaomicron genome to a chow or host glycan-directed glycoside hydrolase. If a Sus gene A lay within the same “directon” (defined as all intervening genes transcribed on the same strand) of a glycoside hydrolase gene B, then B. thetaiotaomicron operon predictions were checked to see whether A and B were likely part of a common operon. Operon associations between glycoside hydrolases (left column) and SusC/D paralogs (right column) are shown for genes upregulated in mice fed a simple sugar-rich diet (green box) or a polysaccharide-rich diet (brown box).

FIG. 37 depicts a schematic illustrating relative expression levels of CPS loci genes showing differential expression in B. thetaiotaomicron grown in vitro and in vivo. Differential expression relative to MM-G is defined using the following criteria: (i) fold difference ≧1.2 using lower 90% confidence bound; (ii) signal difference ≧100; and (iii) upregulated genes (transcripts) called “Present” in ≧66% GeneChip datasets generated from cecal samples or in ≧20% of samples harvested during in vitro growth in a given medium (i.e., at least one of the time points).

FIG. 38 depicts a schematic view of adaptive foraging of glycans by B. thetaiotaomicron. Bacterial consortia assemble on nutrient scaffolds composed of partially digested plant glycans, shed mucus, or exfoliated epithelial cells. These scaffolds interact with one another, and with the intact mucus layer, serve to oppose bacterial washout from the gut bioreactor, and enhance nutrient harvest and exchange with other members of the microbiota. Insets: bacterial attachment to nutrient scaffolds is promoted by glycan-specific outer membrane binding proteins (SusC/D paralogs), induced depending upon the glycan landscape encountered in the gut micro-habitat. If dietary polysaccharides are unavailable, B. thetaiotaomicron forages on mucus glycans.

### DETAILED DESCRIPTION OF THE PREFERRED EMBODIMENTS

I. Methods for Determining Modulation in Gene Expression Resulting from Colonization of the Mammalian Intestine with Components of the Gut Microbiota

Mammals generally, and humans in particular, are home to an incredibly complex and abundant ensemble of microbes. Assembly of components of this microbiota begins at birth. The adult human intestine is home to an almost inconceivable number of micro-organisms. The size of the population—up to 100 trillion—far exceeds that of all other microbial communities associated with our body's surfaces, and is 10-fold greater than the total number of our somatic and germ cells. Thus, it seems appropriate to view ourselves as a composite of many species and our genetic landscape as an amalgam of genes embedded in our H. sapiens genome and in the genomes of our affiliated microbial partners (the ‘microbiome’).

The human gut microbiota can be pictured as a microbial organ placed within a host organ: it is composed of different cell lineages with a capacity to communicate with one another and the host; it consumes, stores and re-distributes energy; it mediates physiologically important chemical transformations; and it can maintain and repair itself through self-replication. The gut microbiome, which may contain ≧100 times the number of genes as the human genome, endows humans with functional attributes we have not had to evolve on our own.

Our relationship with components of this microbiota is often described as ‘commensal’ (one partner benefits, the other is apparently unaffected), as opposed to mutualistic (both partners experience increased fitness). However, use of the term commensal generally reflects our lack of knowledge, or at least an agnostic (noncommittal) attitude about the contributions of most citizens of this microbial society to the fitness of other community members, or ourselves.

The guts of ruminants and termites are well-studied examples of bioreactors ‘programmed’ with anaerobic bacteria charged with the task of breaking down ingested polysaccharides, the most abundant biological polymer on our planet, and fermenting the resulting monosaccharide soup to short chain fatty acids. In these mutualistic relationships, the hosts gain carbon and energy, while their microbes are provided with a rich buffet of glycans and a protected anoxic environment (A. Brune, et al, Curr Opin Microbiol 3, 263 (2000)). The human distal intestine is also an anaerobic bioreactor that harbors the majority of our gut microorganisms: they degrade a varied menu of otherwise indigestible polysaccharides, including plant-derived pectin, cellulose, hemicellulose, and resistant starches.

The adult human GI tract contains all three domains of life - Archaea, Eukarya, and Bacteria. Bacteria living in the human gut achieve the highest cell densities recorded for any ecosystem (W. B. Whitman, et al, Proc. Natl. Acad. Sci. USA. 95, 6578 (1998)). Nonetheless, diversity at the division-level (superkingdom, or deep evolutionary lineage) is among the lowest (P. Hugenholtz, et al, J Bact 180, 4765 (1998)): only 8 of the 55 known bacterial divisions have been identified to date (Fig IA), and of these, five are rare. The divisions that dominate—the Cytophaga-Flavobacterium-Bacteroidetes (CFB, e.g., the genus Bacteroides), and the Firmicutes (e.g., the genera Clostridium and Eubacterium) each comprise ˜30% of bacteria in feces and mucus overlying the intestinal epithelium. Proteobacteria are common but usually not dominant (P. Seksik, et al., Gut 52, 237 (2003)). In comparison, soil, the terrestrial biosphere's GI tract where degradation of organic matter occurs, can contain 20 or more bacterial divisions (J. Dunbar, et al., Appl Environ Microbiol, 68, 3035 (2002)).

Although the effects of pathogenic or other potentially harmful invasive microorganisms have been studied (see for example L. Eckmann, et al, J Biol. Chem., 275, 14084 (2000);. D. A. Relman, Science, 284,1308 (1999); D. A. Relman, Curr. Opin. Immunol., 2, 215 (2000)) little is known about how gut bacteria shape normal human development and physiology. This is due partly to a paucity of defined, experimentally tractable in vivo model systems for examining how nonpathogenic microorganisms regulate host biology.

A mouse model using adult germ-free animals, colonized with Bacteroides thetaiotaomieron, has previously been used to show that this prominent member of the normal distal human and mouse intestinal microbiota regulates production of distal small intestinal (ileal) epithelial fucosylated glycans after it is introduced into germ-free mice, and to delineate how the microbe controls production of these glycans for its own nutritional benefit (L. Bry, et al., Science 273, 1380. (1996); L. V. Hooper, et al, Proc. Natl. Acad. Sci. USA, 96, 9833 (1999)).

Virtually nothing else is known about how indigenous bacteria modulate intestinal gene expression and how this impacts the host's digestive process. It has been discovered that components of the microbiota make significant contributions to nutrient digestion, and to other aspects of gut physiology and maturation. The present invention encompasses (i) methods for testing the impact of components of an animal's gut microbiota on intestinal gene expression, including the effects of specific components of this microbiota on nutrient harvest and uptake, and the pathways used to regulate host storage of energy extracted from the diet; (ii) the discovery that Fiaf, a microbiota-modulated host gene product, is a regulator of host energy storage and it, or its derivatives, or activators of Fiaf gene expression, can be used to promote leanness in various mammalian species, including humans; and (iii) manipulation of the composition of the microbiota can be used to modulate host energy balance.

In order to study the changes in intestinal gene expression orchestrated by members of the microbiota bacteria, germ-free mice were colonized with various bacterial species including Bacteroides thetaiotaomicron. Global intestinal transcriptional responses to colonization were delineated using high-density oligonucleotide arrays and the cellular origins of specific responses established by laser capture microdissection and real-time quantitative RT-PCR. A similar approach has been used in germ-free zebrafish to discover host responses to the microbiota that have been conserved between vertebrate species during the course of evolution, including the response of Fiaf

The results illustrated hereinafter, reveal that components of the human gut microbiota modulate expression of a large number of genes. The genes involved participate in diverse and fundamental physiological functions of the gut, including nutrient absorption, mucosal barrier fortification, and xenobiotic metabolism. The microbial species-selectivity of some of the colonization-associated changes in gene expression emphasizes how human physiology can be impacted by changes in the composition of indigenous microbiota. Furthermore, changes associated with the suckling-weaning transition were elicited in adult mice by B. thetaiotaomicron, suggesting that indigenous intestinal bacteria play an instructive role in postnatal gut development. Coupling defined in vivo models with comprehensive genome-based analyses thus provides a powerful approach for identifying the critical contributions of resident microbes to host biology.

Bacteroides thetaiotaomicron is a genetically-manipulatable anaerobe and was chosen for initial study to define the impact of resident bacteria on intestinal (and host) biology because it is a prominent member of both the adult mouse and human gut microbiota and because it is able to breakdown otherwise indigestible polysaccharides which are prominent components of the human diet, and of the diets of many animal species, including domestic animals. Bacteroides thetaiotaomicron's prodigious capacity for digesting otherwise indigestible dietary polysaccharides is reflected in the fully sequenced 6.3 Mb genome of the type strain (ATCC 29148; originally isolated from the feces of a healthy adult human) (J. Xu, et al., Science 299, 2074 (2003)). Its ‘glycobiome’ contains the largest ensemble of genes involved in acquiring and metabolizing carbohydrates yet reported for a sequenced bacterium, including 163 paralogs of two outer membrane proteins (SusC, SusD) that bind and import starch (J. A. Shipman, et al, J Bacteriol 182, 5365 (2000)), 226 predicted glycoside hydrolases, and 15 polysaccharide lyases (http://afinb.cnrs-mrs.fr/CAZY/). By contrast, our 2.85 Gb human genome only contains 98 known or putative glycoside hydrolases, and is deficient in the enzyme activities required for degradation of xylan, pectin, and arabinose-containing polysaccharides that are common components of dietary fiber.

Colonization of adult GF mice with B. thetaiotamicron produced a prominent decrease in expression of fasting-induced adipose factor (Fiaf), previously known to be expressed in liver and fat (S. Kersten, et al., J. Biol. Chem. 275, 28488 (2000)) but not known to be regulated by microbes in any tissue, or to be selectively regulated by microbes in the host intestine. Moreover, qRT-PCR analysis of RNA isolated from laser capture microdissected villus epithelium and villus mesenchyme revealed that Fiaf suppression by B. thetaiotamicron occurred in the epithelium. Microbial regulation of intestinal and villus epithelial expression of Fiaf has not been described previously. In addition, qRT-PCR analysis of intestinal Fiaf expression during postnatal period disclosed that the gene is induced in GF mice during the suckling-weaning transition. Induction does not occur in CONV-R animals, resulting in significantly lower levels of Fiaf mRNA in adult CONV-R versus GF (see FIG. 20). During the suckling-weaning transition, the diet switches from lipid/lactose-rich mother's milk to low fat/polysaccharide-rich chow, with coincident expansion of the microbiota and a shift from facultative to obligate anaerobes (e.g., Bacteroides). These developmental studies suggested that Fiaf could provide a signal that links the microbiota with a change in host energy partitioning. The significant repression of Fiaf found following colonization of adult GF mice with B. thetoaiotaomicron illustrated further hereinafter are indicative of a previously unappreciated mechanism by which a resident gut bacterium, contributes to energy homeostasis.

Additionally, the applicants have found that B. thetaiotamicron colonization elicited a concerted response involving enhanced expression of four genes involved in the breakdown and processing of dietary lipids. mRNAs encoding pancreatic lipase related protein-2 (PLRP-2) and colipase increased an average of 4- and 9-fold, respectively (Tables 1 and 2). PLRP-2 hydrolyzes tri- and diacylglycerols, phospholipids and galactolipids. Colipase augments the activity of PLRP-2 as well as triglyceride lipase (M. E. Lowe, et al., J. Biol. Chem. 273, 31215 (1998)). In addition, there was (i) a 4-6-fold increase in L-FABP mRNA, which encodes an abundant cytosolic protein involved in fatty acid trafficking within enterocytes, and (ii) an induction of apolipoprotein AIV, a prominent component of triglyceride-rich lipoproteins (chylomicrons, VLDL) secreted from the basolateral surfaces of enterocytes (Table 1 below).

TABLE 1
Colonization-associated changes in distal small intestinal gene expression

| | | GenBank/TIGR | average |
|---|---|---|---|
| Gene | function | Reference | fold Δ |
| Nutrient Uptake and | | | |
| Metabolism carbohydrates | | | |
| Na+/glucose cotransporter | glucose uptake | AF163846 | +2.4 |
| (SGLT1) | | | |
| lactase phlorizin-hydrolase | lactose hydrolysis | AA521747 | −2.2 |
| lipids | | | |
| pancreatic lipase-related | lipid metabolism | M30687 | +4.1 |
| protein 2 | | | |
| colipase | lipid metabolism | AA611440 | +9.4 |
| liver fatty acid binding protein | lipid metabolism | Y14660 | +4.0, +5.6 |
| apolipoprotein A-IV | lipid metabolism | M13966 | +2.2 |
| fasting-induced adipose factor | regulation of lipid metabolism | AF278699 | −9.0 |
| phospholipase B | lipid metabolism | TC38683 | −2.2 |
| CYP27 | cholesterol 27-hydroxylation | TC25974 | −2..2 |
| metals | | | |
| high-affinity copper | copper uptake | AA190119 | +2.6 |
| transporter | | | |
| metallothionein I | Cu/Zn sequestration | V00835 | −4.6, −6.1 |
| metallothionein II | Cu/Zn sequestration | K02236 | −5.7, −6.3 |
| ferritin heavy chain | iron sequestration | M24509 | −4.5 |
| cellular energy production | | | |
| isocitrate dehydrogenase | citric acid cycle | U68564 | +2.4 |
| subunit | | | |
| cytochrome c oxidase subunit 1 | mitochondrial electron transport | TC106691 | +2.4 |
| succinyl CoA transferase | ketone body utilization | TC18674 | +2.0 |
| transketolase | Pentose phosphate pathway | u05809 | +2.4 |
| phosphogluconate | Pentose phosphate pathway | C81475 | +2.8 |
| dehydrogenase | | | |
| malate oxidoreductase | malate-asparate shuttle | J02652 | +6.0 |
| asparate aminotransferase | malate-asparate shuttle | J02623 | +2.5 |
| hormonal/maturational | | | |
| responses | | | |
| adenosine deaminase | adenosine inactivation | M10319 | +2.3 |
| omithine decarboxylase | regulation of polyamine levels | U52823 | +2.4 |
| antizyme | | | |
| 15-hydroxyprostaglandin | prostaglandin inactivation | U44389 | −3.2 |
| dehydrogenase | | | |
| GARG-16 | response to glucocorticoid | U43084 | −4.0, −4.5 |
| | production | | |
| FKBP51 | component of steroid receptor | U16959 | −3.8 |
| | complex | | |
| androgen-regulated vas | steroidogenesis | J05663 | −3.3, −3.4 |
| deferens protein | | | |
| short chain dehydrogenase | steroid/retinoid metabolism | AF056194 | −2.2, −2.8 |
| heat-stable antigen | hematopoietic differentiation | X53825 | +3.0 |
| | marker | | |
| Mucosal barrier function | | | |
| decay-accelerating factor | complement inactivation | D63679 | +5.2 |
| polymeric Ig receptor | transepithelial IgA transport | U06431 | +2.3 |
| small proline-rich protein 2a | crosslinking protein | AJ005559 | +10.6, |
| | | | +102 |
| serum amyloid A protein | acute phase response | U60437 | +2.8, +5.4 |
| CRP-ductinα (MUCLIN) | mucin-like protein | U37438 | +2.4 |
| zeta proteasome chain | antigen presentation | AF019661 | +2.8 |
| anti-DNA IgG light chain | | U55583 | +2.5 |
| Detoxification/drug resistance | | | |
| glutathione S-transferase | GSH conjugation to | L06047 | −2.4 |
| | electrophiles | | |
| P-glycoprotein (mdrla) | export of GSH-conjugated | M33581 | −4.6 |
| | compounds | | |
| CYP2D2 | 4-hydroxylase | TC36686 | −2.6 |
| Enteric nervous system/ | | | |
| muscular layers | | | |
| L-glutamate transporter | glutamate uptake | U73521 | +4.4 |
| L-glutamate decarboxylase | GABA production | M55253 | +2.2 |
| vesicle-associated protein-33 | neurotransmitter release | AF157497 | +2.2 |
| cysteine-rich protein 2 | cGMP kinase I target | AA028770 | +3.2 |
| smooth muscle (enteric) | contractility | M26689 | +2.3 |
| gamma actin | | | |
| SM-20 | growth-factor responsive gene | TC33445 | +4.8 |
| Calcium channel5 subunit | calcium channel regulation | AJ272046 | −2.2 |
| angiogenesis | | | |
| angiogenin-4 | unknown | SEQ ID NO. 29 | +10.9 |
| angiogenin-related protein | unknown | U22519 | +6.4 |
| angiogenin family¹ | | | +2.4, +6.0, |
| | | | +7.0 |
| cytoskeleton/extra-cellular | | | |
| matrix | | | |
| gelsolin | actin binding protein | J04953 | +7.9 |
| destrin | actin depolymerizing factor | W17549 | +3.0 |
| alpha cardiac actin | contractility | M15501 | +3.4 |
| endoB cytokeratin | intermediate filament protein | m11686 | +3.0 |
| fibronectin | extracellular matrix protein | M18194 | +2.9, +3.2 |
| proteinase inhibitor 6 | serine protease inhibitor | U25844 | +2.6 |
| mpgc60 | serine protease inhibitor | Y11505 | +2.5 |
| alpha 1 type 1 collagen | extracellular matrix protein | X06753 | +2.2, +4.7 |
| signal transduction | | | |
| Pten | protein/lipid phosphatase | U92437 | +3.2 |
| gp106 (TB2/DP1) | unknown | U28168 | +6.9 |
| rac2 | ras-related GTP-binding protein | X53247 | +7.0 |
| Semcap2 | SemaF-associated protein | AF061262 | −2.9 |
| serum and glucocorticoid- | serine/threonine protein kinase | AF139638 | −2.6 |
| regulated kinase | | | |
| STE20-like protein kinase | serine/threonine protein kinase | AA154321 | +2.6 |
| B-cell myeloid kinase | unknown | J03023 | +2.1 |
| general cellular functions | | | |
| glutathione reductase | maintenance of reduced | X76341 | +2.9 |
| | glutathione | | |
| calmodulin | calcium homeostasis | M27844 | +2.2 |
| e1F3 subunit | translation initiation | U70736 | +2.7 |
| hsc70 | stress response | U73744 | +2.9 |
| oligosaccharyl transferase | protein N-glycosylation | U84211 | +3.4 |
| subunit | | | |
| fibrillarin | ribosomal RNA processing | Z22593 | +2.4 |
| H+-transporting ATPase | intracellular organelle | AA108559 | +2.9 |
| | acidification | | |
| Msec23 | component of the COPII | AA116735 | +2.8 |
| | complex | | |
| vacuolar protein sorting 35 | membrane protein recycling | U47024 | +2.4 |

Additionally, the applicants have found that colonization produces changes in expression of four genes involved in dietary metal absorption. A high affinity epithelial copper transporter (CRT1) mRNA was increased, while metallothionein-I, metallothionein-II, and ferritin heavy chain mRNAs were decreased (Table 1). These changes suggest that colonization engenders increased capacity to absorb heavy metals (e.g., via CRT1) and a concomitant decreased capacity to sequester them within cells (MT-I/II, ferritin). This implies greater host demand for these compounds, either due to increased utilization by the host's own metabolic pathways or to competition with the microbe. These changes in gene expression (plus those of several other mRNAs discussed below), were independently validated by qRT-PCR (C. A. Heid, et al., Genome Res. 6, 986 (1996) (see Table 2 below).

TABLE 2
Real-time quantitative RT-PCR studies of colonization-
associated changes in gene expression

| | Fold - difference |
|---|---|
| | (relative to germ- |
| Gene | free) |
| Na+/glucose cotransporter (SGLT1) | 2.6 ± 0.9 |
| colipase | 6.6 ± 1.9 |
| liver fatty acid binding protein (L-FABP) | 4.4 ± 1.4 |
| metallothionein I (MT-I) | −5.4 ± 0.7 |
| polymeric immunoglobulin receptor (pIgR) | 2.6 ± 0.7 |
| decay accelerating factor (DAF) | 5.7 ± 1.5 |
| small proline-rich protein 2a (sprr2a) | 205 ± 64 |
| multi-drug resistance protein (mdrla) | −3.8 ± 1.0 |
| glutathione S-transferase (GST) | −2.1 ± 0.1 |
| lactase-phlorizin hydrolase | −4.1 ± 0.6 |
| adenosine deaminase (ADA) | 2.6 ± 0.6 |
| angiogenin-4 | 9.1 ± 1.8 |

Additionally, the applicants have found that B. thetaiotaomicron colonization produces effects that enhance intestinal barrier function. An intact mucosal barrier is critical for accommodating the vast population of resident intestinal microbes. Its disruption can provoke an immune response that is deleterious to the host and to the stability of microbiota, leading to pathologic states such as inflammatory bowel disease (reviewed in, for example, P. G. Falk, et al, Microbiol. Mol. Biol. Rev. 62, 1157 (1998); P. J. Sansonetti, Nat Rev Immunol., 4, 953 (2004)).

B. thetoaiotaomicron produces no detectable inflammatory response, as judged by histologic surveys (L. Bry, et al., Science 273, 1380 (1996)) and no discernible induction (or repression) of the many genes, represented on the DNA microarrays, that are involved in these types of inflammatory responses. An influx of IgA-producing B-cells does occur in the ileal mucosa 10 days after introduction of B. thetaiotaomicron; similar commensal-induced IgA responses have been shown to be T-cell independent and to enforce barrier integrity (A. J. Macpherson, et al., Science 288, 2222 (2000)).

Genes involved in barrier function account for 10% (7/71) of the changes in gene expression observed with B. thetaiotaomicron colonization. DNA microarray and qRT-PCR analyses revealed that the influx of IgA producing B-cells is accompanied by increased expression of the polymeric immunoglobulin receptor (pIgR) that transports IgA across the epithelium (Tables 1, 2). There is also augmented expression of the CRP-ductin gene, encoding both a component of the protective mucus layer overlying the epithelium (MUCLIN; R. C. DeLisle, et al., Am. J Physiol. 275, G219 (1998)) and a putative receptor for trefoil peptides that participate in fortification/healing of the intestinal mucosa (L. Thim, et al., Regul. Pept. 90, 61 (2000)). Additionally, there is increased expression of decay accelerating factor (DAF), an apical epithelial surface protein that inhibits complement-mediated cytolysis (M. E. Medof, et al, J. Exp. Med. 165, 848 (1987)). Coincident enhancement of pIgR, MUCLIN, and DAF expression should not only help prevent bacteria from crossing the epithelial barrier, but should also prevent mucosal damage that may ensue from microbial activation of complement components present in intestinal secretions.

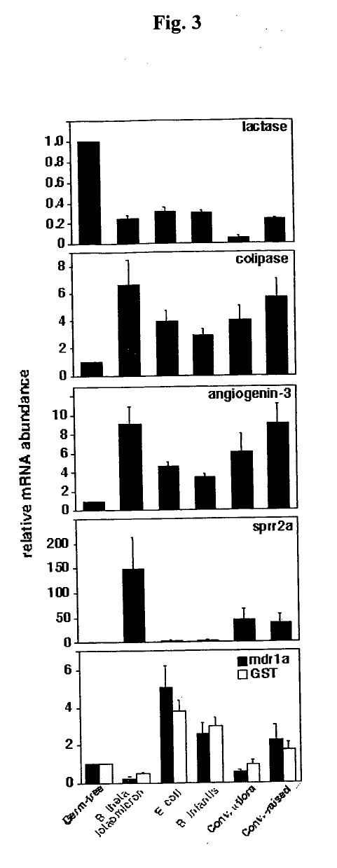

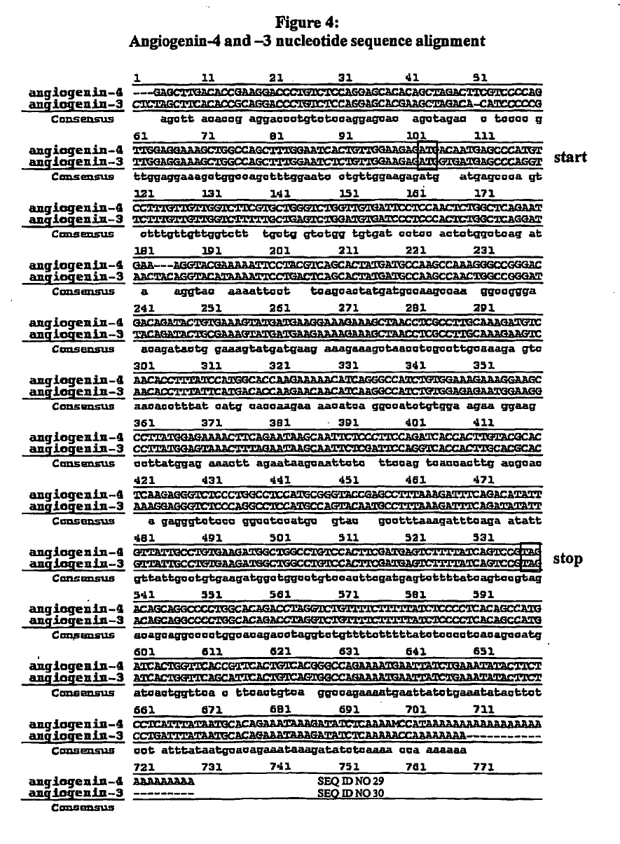

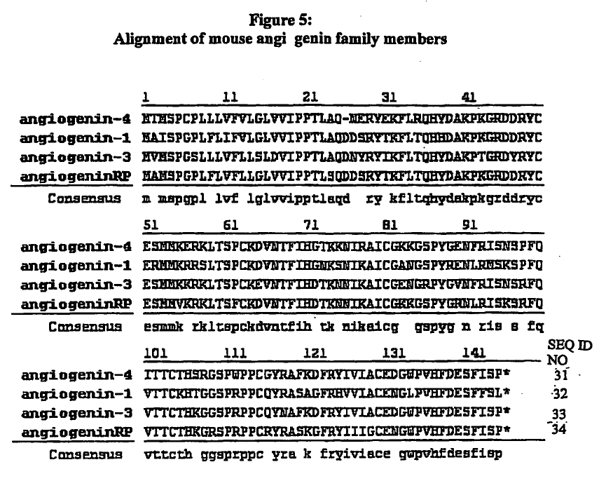

The most pronounced response to B. thetaiotaomicron was an increase in small proline-rich protein-2 (sprr2a) mRNA (Table 1). qRT-PCR analysis established that there wass a 205±64-fold elevation in this mRNA with colonization (Table 2), and that this response had microbial specificity (FIG. 3). Sprr2a is a member of a family of proteins associated with terminal differentiation of squamous epithelial cells. Sprrs contribute to the barrier functions of squamous epithelia, both as a component of the comified cell envelope, and as cross-bridging proteins linked to desmosomal desmoplakin, a prominent desmosomal constituent (P. M. Steinert, et al., Mol. Biol. Cell 10. 4247 (1999)). Colonization did not produce a notable change (i.e. two-fold or more), in the expression of genes encoding other proteins linked to desmosomes (desmoplakin, plakoglobin, plakophilin, plectin), or tight junctions (ZO-1, occludin).

Sprr2a expression in the intestine and its microbial regulation are novel findings. The critical contribution of Sprr2a to the squamous epithelial barrier and the dramatic response of sprr2a expression to B. thetaiotaomicron together suggest that this protein plays an important role in intestinal barrier function. It is therefore a particularly suitable target for further investigation in accordance with the invention, in particular by evaluating the biochemical pathway in which Sprr2a participates in intestinal barrier functions, the mechanism by which B. thetaiotaomicron regulates Sprr2a expression and the utility of using B. thetaiotaomicron as a probiotic to enhance intestinal barrier function.

Using the method of the invention, it has been found that colonization results in increased expression of angiogenin-4 which resembles angiogenin-3, a secreted protein with demonstrated angiogenic activity (X. Fu, et al., Mol. Cell Biol. 17, 1503 (1997), X. Fu, et al., Growth Factors 17, 125 (1999)). The 11-fold increase in expression of the angiogenesis factor recognizable by amplification using primers of SEQ ID NO 12 and SEQ ID NO 25, which is angiogenin-4 (Table 1, 2) upon B. thetaiotaomicron colonization represents a novel mode of regulation for this or other new putative angiogenesis factors, and so may be the subject of further investigation in accordance with the invention. Laser capture microdissection (LCM) experiments described below have delineated the cellular origins of this response.

The gut is the site of first contact of innumerable ingested toxins and xenobiotics. The relative contributions of luminal bacteria and the epithelium to detoxification and metabolism of these compounds has been difficult to delineate in conventionally-raised mammals. It has been found that colonization of germ-free mice with B. thetaiotaomicron results in reduced expression of several genes involved in these processes (Table 1). There is a decrease in the host mRNA encoding glutathione S-transferase, which detoxifies a variety of electrophiles, and a corresponding decrease in multi-drug resistance protein-1 (Mdr-1), which exports glutathione-conjugated compounds from the epithelium (R. W. Johnstone, et al., Trends Biochem. Sci. 25, 1 (2000)). Expression of CYP2D2 (debrisoquine hydroxylase) involved in oxidative drug metabolism in humans (M. lngelman-Sundberg, et al., Trends Pharmacol. Sci. 20, 342 (1999)), also declines with colonization. A genetic polymorphism that produces a deficiency in this cytochrome P-450 is common in humans and associated with altered oxidative drug metabolism (M. Ingelman-Sundberg, et al., Trends Pharmacol. Sci. 20, 342 (1999)). The reduced expression of these three host genes suggests that components of the microbiota, such as B. thetaiotaomicron, contribute to the detoxification of compounds that could be deleterious to the host. This indicates that a component of the normal intestinal microbiota can modulate host genes involved in drug metabolism, and underscore how variations in such metabolism between individuals may arise from differences in the composition of their resident intestinal microbial communities. Consequently, evaluation of the effect of indigenous gut bacterial species on expression of these genes using the method of the invention may be helpful—both as means for testing the role of the microbiota in metabolism of drugs, and for identifying novel microbial biotransformation activities that could be used to develop more or less active forms of drugs.

The motility of the intestine is regulated by its enteric nervous system (ENS). The relative contributions of intrinsic and extrinsic factors to ENS activity are poorly understood, despite the fact that irritable bowel syndrome, which involves dysregulated motor activity, is a major health problem. The impact of components of the microbiota, such as B. thetaiotaomicron, on gut physiology extends to genes expressed in the enteric nervous system (ENS) and in the muscular layers. mRNAs encoding the L-glutamate transporter and L-glutamate decarboxylase, which converts glutamate to GABA, are both increased, suggesting a colonization-associated effect on the glutamatergic neurons of the ENS (M. T. Liu, et al., J. Neurosci. 17, 4764 (1997)). Enhanced expression of vesicle-associated protein-33, a synaptobrevin-binding protein involved in neurotransmitter release (P. A. Skehel, et al., Proc. Natl. Acad. Sci. U.S.A. 97, 1101 (2000)) is also observed. There is a concomitant increase in two muscle-specific mRNAs: enteric y-actin and cysteine-rich protein 2. Previous electrophysiological studies of germ-free and conventionally-raised animals have suggested that the microbiota plays a role in gut motility (E. Husebye, et al., Dig. Dis. Sci. 39, 946 (1994)). The method of the invention can provide molecular details about how resident gut microbes, such as B. thetaiotaomicron, may act to modulate intestinal motility.

Expression profiling revealed surprisingly that colonization of adult germ-free mice with B. thetaiotaomicron elicits other responses that mimic changes that normally occur in the maturing intestine of conventionally-reared animals. Expression of lactase, which hydrolyzes the principal milk sugar (lactose), normally declines during the weaning period (S. D. Krasinski, et al., Am. J Physiol. 267, G584 (1994)). Colonization of adult germ-free mice with B. thetaiotaomicron produces a decrease in ileal lactase mRNA (Table 1, 2). Adenosine deaminase (ADA) and polyamines (spermine, spermidine) play important roles in postnatal intestinal maturation (G. D. Luk, et al., Science 210, 195 (1980); J. M. Chinsky, et al., Differentiation 42, 172 (1990)). It has been found that B. thetaiotaomicron colonization produces an increase in mRNAs encoding ADA and ornithine decarboxylase (ODC) antizyme. The antizyme, whose expression is affected by polyamine levels, is a critical regulator of ODC turnover (J. Nilsson, et al., Eur. J Biochem. 250, 223 (1997)); an increase in antizyme mRNA levels therefore suggests that colonization influences ileal polyamine synthesis. These data demonstrate that genes controlling synthesis of two classes of regulators of gut maturation, adenosine and polyamines, are themselves modulated by a component of the microbiota, leading to the idea that bacteria serve as upstream effectors of a cascade that affects gut maturation. Some changes in gut maturation associated with the suckling-weaning transition are thought to be regulated by increases in glucocorticoids (S. J. Henning, et al., in Physiology of the Gastrointestinal Tract, L. R. Johnson, Ed. (Raven Press, New York. 1994), pp. 584-586)). B. thetaiotaomicron colonization as described hereinafter was accompanied by reduced expression of two genes whose transcription is known to be suppressed by glucocorticoids: I5-hydroxyprostaglandin dehydrogenase (M. D. Mitchell, et al., Prostaglandins Leukot. Essent. Fatty Acids 62, 1 (2000)) and glucocorticoid-attenuated response gene-16 (J. B. Smith, et al., J. Biol. Chem 270, 16756 (1995)). Furthermore, there was reduced expression of another gene whose product interacts with nuclear hormone receptor family members, the immunophilin FKBP5I (S. C. Nair, et al., Mol. Cell. Biol. 17. 594 (1997)).

As mentioned above, the applicants have found that a particular member of the angiogenin family, whose gene is amplifiable using primers of SEQ ID NO 12 and 25 (Table 3 below) and is expressed in mouse intestine, is novel. Thus, this protein and the gene encoding it forms a further aspect of the invention.

TABLE 3

| | | | | |
|---|---|---|---|---|
| | | SEQ | | SEQ |
| | | ID | | ID |
| gene name | forward primer | NO | reverse primer | NO |

| | | | | |
|---|---|---|---|---|
| Na+/glucose | 5′-CAGAGACCCCATTACTGGAG | 1 | 5′-TCGTTGCACAATGACCTGATC | 14 |
| cotransporter | ACA | | | |
| (SGLT1) | | | | |
| colipase | 5-TGACACCATCCTGGGCATT | 2 | 5′-ACACCGGTAGTAAATCCCATAA | 15 |
| | | | AGG | |
| liver fatty acid | 5′-CTCCGGCAAGTACCAATTGC | 3 | 5′-TGTCCTTCCCTTTCTGGATGAG | 16 |
| binding protein | | | | |
| (L-FABP) | | | | |
| metallothioneinI | 5′-ATGTGCCCAGGGCTGTGT | 4 | 5′-AACAGGGTGGAACTGTATAGGA | 17 |
| (MT-I) | | | AGAC | |
| polymeric immunoglobulin | 5′-CTTCCCTCCTGTCCTCAGAGGT | 5 | 5′-GGCGTAACTAGGCCAGGCTT | 18 |
| receptor (pIgR) | | | | |
| decay accelerating | 5′-CAACCCAGGGTACAGGCTAGTC | 6 | 5′-GGTGGCTCTGGACAATGTAT | 19 |
| factor (DAF) | | | TTC | |
| small proline-rich | 5′-CCTTGTCCTCCCCAAGCG | 7 | 5′-AGGGCATGTTGACTGCCAT | 20 |
| protein 2a (sprr2a) | | | | |
| multi-drug resistance | 5′-GCCGCTTCTTCCAAAGTCTACA | 8 | 5′-CGTGTCTCTACTCCCGGTTTCC | 21 |
| protein (mdrla) | | | | |
| glutathione S-transferase | 5′-CATCCAGCTCCTAGAAGCCATT | 9 | 5′-GGGTTGCAGGAACTTCTTAATT | 22 |
| (GST) | | | GTA | |
| lactase-phlorizin | 5′-TTGAATGGGCCACAGGCT | 10 | 5′-AGCGGACTATGGAGGCGTAG | 23 |
| hydrolase | | | | |
| adenosine deaminase | 5′-GCGCAGTAAAGAATGGCATTC | 11 | 5′-CTGTCTTGAGGATGTCCACAGC | 24 |
| (ADA) | | | | |
| angiogenin-4 | 5′-TCGATTCCAGGTCACCACTTG | 12 | 5′-CACAGGCAATAACAATATATCT | 25 |
| | | | GAAATCT | |
| glyceraldehyde | 5′-TGGCAAAGTGGAGATTGTTGCC | 13 | 5′-AAGATGGTGATGGGCTTCGCG | 26 |
| 3-phosphate | | | | |
| dehydrogenase | | | | |

A further aspect of the invention provides a protein of SEQ ID NO 29 as shown in FIG. 4 hereinafter, or an allelic variant thereof or a protein which has at least 85% amino acid sequence identity with SEQ ID NO 29. In particular, the invention provides a protein of SEQ ID NO. 29. In yet a further aspect, the invention provides a nucleic acid that encodes a protein as described above. These proteins are useful as a target for the screening process of the invention.

II. Modulation of Fiaf and the Gastrointestinal Microbiota as a Means to Control Energy Storage in a Subject

The applicants have discovered, as detailed in section I, that B. thetaioatomicron alone, or a more complex microbiota, modulates expression of a subject's Fiaf. It has further been discovered, as detailed in the examples below, that the microbiota regulates a subject's energy storage in part by selectively suppressing a subject's gastrointestinal transcription of Fiaf Referring to FIG. 18, the gut microbiota effects a subject's energy storage through Fiaf by coordinating increased digestion of dietary polysaccharides, increased hepatic lipogenesis and increased LPL activity in adipocytes, thereby promoting storage of calories harvested from the diet to fat. Taking advantage of these discoveries, the present invention provides compositions and methods that may be employed for decreasing body fat and for promoting weight loss in a subject.

(A) Modulation of Fiaf

One aspect of the present invention provides a method to regulate fat storage and weight loss in a subject by modulating the amount of or the activity of Fiaf. To decrease body fat and promote weight loss, the amount of or the activity of Fiaf is increased in the subject.

In one embodiment, Fiaf may be increased by administering a suitable Fiaf polypeptide to the subject. Typically, a suitable Fiaf polypeptide is one that can substantially inhibit LPL when administered to the subject. A number of Fiaf polypeptides known in the art are suitable for use in the present invention. Generally speaking, the Fiaf polypeptide is from a mammal. By way of non limiting example, suitable Fiaf polypeptides and nucleotides are delineated in Table Z

TABLE Z

| Species | PubMed Ref. |
|---|---|
| Homo sapiens | NM_139314 |
| | NM_016109 |
| Mus musculus | NM_020581 |
| Rattus norvegicus | NM_199115 |
| Sus scrofa | AY307772 |
| Bos taurus | AY192008 |
| Pan troglodytes | AY411895 |

In certain aspects, a polypeptide that is a homolog, ortholog, mimic or degenerative variant of a Fiaf polypeptide is also suitable for use in the present invention. In particular, the subject polypeptide will typically inhibit LPL when administered to the subject.

A number of methods may be employed to determine whether a particular homolog, mimic or degenerative variant possesses substantially similar biological activity relative to a Fiaf polypeptide. Specific activity or finction may be determined by convenient in vitro, cell-based, or in vivo assays, such as measurement of LPL activity in white adipose tissue or in the heart. In order to determine whether a particular Fiaf polypeptide inhibits LPL, the procedures detailed in lo the examples may be followed.

In addition to having a substantially similar biological function, a homolog ortholog, mimic or degenerative variant suitable for use in the invention will also typically share substantial sequence similarity to a Fiaf polypeptide. In addition, suitable homologs, ortholog, mimic or degenerative variants preferably share at least 30% sequence homology with a Fiaf polypeptide, more preferably, 50%, and even more preferably, are greater than about 75% homologous in sequence to a Fiaf polypeptide. Alternatively, peptide mimics of Fiaf could be used that retain critical molecular recognition elements, although peptide bonds, side chain structures, chiral centers and other features of the parental active protein sequence may be replaced by chemical entities that are not native to Fiaf protein yet, nevertheless, confer activity.

In determining whether a polypeptide is substantially homologous to a Fiaf polypeptide, sequence similarity may be determined by conventional algorithms, which typically allow introduction of a small number of gaps in order to achieve the best fit. In particular, “percent homology” of two polypeptides or two nucleic acid sequences is determined using the algorithm of Karlin and Altschul [(Proc. Natl. Acad. Sci. USA 87, 2264 (1993)]. Such an algorithm is incorporated into the NBLAST and XBLAST programs of Altschul, et al. (J. Mol. Biol. 215, 403 (1990)). BLAST nucleotide searches may be performed with the NBLAST program to obtain nucleotide sequences homologous to a nucleic acid molecule of the invention. Equally, BLAST protein searches may be performed with the XBLAST program to obtain amino acid sequences that are homologous to a polypeptide of the invention. To obtain gapped alignments for comparison purposes, Gapped BLAST is utilized as described in Altschul, et al. (Nucleic Acids Res. 25, 3389 (1997)). When utilizing BLAST and Gapped BLAST programs, the default parameters of the respective programs (e.g., XBLAST and NBLAST) are employed. See http://www.ncbi.nlm.nih.gov for more details.

Fiaf polypeptides suitable for use in the invention are typically isolated or pure and are generally administered as a composition in conjunction with a suitable pharmaceutical carrier, as detailed below. A pure pplypeptide constitutes at least about 90%, preferably, 95% and even more preferably, at least about 99% by weight of the total polypeptide in a given sample.

The Fiaf polypeptide may be synthesized, produced by recombinant technology, or purified from cells using any of the molecular and biochemical methods known in the art that are available for biochemical synthesis, molecular expression and purification of the Fiaf polypeptides [see e.g., Molecular Cloning, A Laboratory Manual (Sambrook, et al. Cold Spring Harbor Laboratory), Current Protocols in Molecular Biology (Eds. Ausubel, et al., Greene Publ. Assoc., Wiley-Interscience, New York)].

Expression vectors that may be effective for the expression of Fiaf polypeptides include, but are not limited to, the PCDNA 3.1, EPITAG, PRCCMV2, PREP, PVAX, PCR2-TOPOTA vectors (Invitrogen, Carlsbad Calif.), PCMV-SCRIPT, PCMV-TAG, PEGSHIPERV (Stratagene, La Jolla Calif.), and PTET-OFF, PTET-ON, PTRE2, PTRE2-LUC, PTK-HYG (Clontech, Palo Alto Calif.). Fiaf polypeptides may be expressed using (i) a constitutively active promoter, (e.g., from cytomegalovirus (CMV), Rous sarcoma virus (RSV), SV40 virus, thymidine kinase (TK), or P β-actin genes), (ii) an inducible promoter (e.g., the tetracycline-regulated promoter (Gossen, et al., Proc. Natl. Acad. Sci. USA, 89, 5547 (1992); M. Gossen, et al., Science, 268, 1766 (1995); F. M., Rossi, et al., Curr. Opin. Biotechnol. 9, 451 (1998), commercially available in the T-REX plasmid (Invitrogen)); the ecdysone-inducible promoter (available in the plasmids PVGRXR and PIND; Invitrogen); the FK506/rapanmycin inducible promoter; or the RU486/mifepristone inducible promoter (F.M. Rossi, et aL, supra)), or (iii) a tissue-specific promoter or the native promoter of the endogenous gene encoding Fiaf from a normal individual.

Commercially available liposome transformation kits (e.g., the PERFECT LIPID TRANSFECTION KIT, available from Invitrogen) allow one with ordinary skill in the art to deliver Fiaf polynucleotides to target cells in culture, and require minimal effort to optimize experimental parameters. Alternatively, transformation is performed using the calcium phosphate method (F. L. Graham, et al., Virology, 52, 456 (1973), or by electroporation (E. Neumann, et al., EMBO J, 1, 841 (1982)).

A Fiaf peptide can be synthesized using traditional solid-phase methods.

In another alternative of this embodiment, an agent can be delivered that specifically activates Fiaf expression: this agent could represent a natural or synthetic compound that directly activates Fiaf gene transcription, or indirectly activates expression through interactions with components of host regulatory networks that control Fiaftranscription. For example, such an agent could be identified by screening natural product and/or chemical libraries using the gnotobiotic zebrafish model described below as a bioassay.

In another embodiment, a chemical entity could be used that interacts with Fiaf targets such as LPL to reproduce the effects of Fiaf (e.g., in this case inhibition of LPL activity).

In another alternative of this embodiment, Fiaf expression and/or activity may be increased by administering a Fiaf agonist to the subject. In one preferred embodiment, the Fiaf agonist is a peroxisome proliferator-activated receptor (PPARs) agonist. Suitable PPARs include PPARα, PPARβ/δ, and PPARγ. Fenofibrate is another suitable example of a Fiaf agonist. Additional suitable Fiaf agonists and methods of administration are further described in Manards, et al., J. Biol Chem, 279, 34411 (2004), and U.S. Patent Publication No. 2003/0220373, which are both hereby incorporated by reference in their entirety.

In yet another a further alternative of this embodiment, Fiaf is increased in a subject by altering the microbiota population in the subject's gastrointestinal tract such that the microbial-mediated suppression of Fiaf in the subject is decreased. Suitable methods for altering the microbial population are described in detail in section II (B).

(B) Alteration of the Gastrointestinal Microbiota Population

Another aspect of the present invention provides a method to regulate fat storage and weight loss in a subject by altering the microbial population in the subject's gastrointestinal tract. To decrease body fat and promote weight loss, the microbiota is altered such that at least one microbial-mediated signaling pathway in the subject that regulates energy storage is either substantially inhibited or stimulated, whereby stimulating or inhibiting the signaling pathway causes a decrease in body fat or promotes weight loss in the subject. In one embodiment, the microbiota population may be altered such that microbial-mediated transcriptional suppression of a LPL inhibitor, such as Fiaf, is decreased in the subject and results in a decrease of triglyceride storage in the adipocytes of the subject. In a certain embodiment, Fiaf is selectively increased only in the gastrointestinal tract of the subject. In yet another embodiment, the microbiota population may be altered such that a signaling pathway that regulates hepatic lipogenesis is substantially inhibited, thereby resulting in a decrease of triglyceride storage in the adipocytes of the subject. In one embodiment, hepatic lipogenesis is substantially inhibited as a result of a decrease in microbial processing of dietary polysaccharides.

Accordingly, in one embodiment, the subject's gastrointestinal microbial population is altered so as to decrease body fat and promote weight loss in the subject. In one alternative of this embodiment, the presence of microbes that suppress Fiaf transcription may be decreased. In one alternative of this embodiment, the presence of saccharolytic microbes, such as Bacteroides, is decreased. (Saccharolytic microbes typically degrade complex, otherwise indigestible dietary polysaccharides that the subject cannot.) In another alternative embodiment, the presence of microbes that ferment sugars to short chain fatty acids is decreased. In still another embodiment, the presence of microbes that increase the uptake of microbial and diet-derived monosaccharides (e.g., glucose, fructose and galactose) by the host is decreased.

To decrease the presence of any of the microbes detailed above, methods generally known in the art may be utilized. In one embodiment, a suitable probiotic is administered to the subject. Generally speaking, suitable probiotics include those that alter the representation or biological properties of microbiota populations that are involved in a subject's uptake of energy. By way of non-limiting example, suitable probiotics include Lactobacillus, Acidophillus and Bifidobacteria, each of which is commercially available from several sources. In another embodiment, microbes that induce Fiaf expression in the subject's gastrointestinal tract may be administered to the subject. In yet another embodiment, selective reduction in the representation of components of the microbiota, such as saccharolytic bacteria, is achieved by administering an antibiotic to the subject. In yet another embodiment, selective reduction in the representation of components of the microbiota, such as saccharolytic bacteria, is achieved with antibiotics.

In yet another embodiment, a subject may be administered a diet that alters the microbiota population so as to decrease body fat and promote weight loss in the subject.

(C) Combination Therapy

Another aspect of the invention encompasses a combination therapy to regulate fat storage and weight loss in a subject. In one embodiment, the invention encompasses a composition for decreasing body fat or for promoting weight loss. Typically, the composition comprises a Fiaf polypeptide and an agent that alters the microbiota population in a subject's gastrointestinal tract such that microbial-mediated transcriptional suppression of a LPL inhibitor in the subject is decreased. Suitable Fiaf polypeptides and agents that alter the microbiota population are detailed above.

In other embodiments, any of the proteins or polypeptides, agonists, of the invention as detailed in section II may be administered in combination with other appropriate therapeutic agents. Selection of the appropriate agents for use in combination therapy may be made by one of ordinary skill in the art, according to conventional pharmaceutical principles. Generally speaking, agents will include those that decrease body fat or promote weight loss by a mechanism other the mechanisms detailed herein. In one embodiment, acarbose may be administered with any compound described herein. Acarbose is an inhibitor of α-glucosidases and is required to break down carbohydrates into simple sugars within the gastrointestinal tract of the subject. In another embodiment, an appetite suppressant such as an amphetamine or a selective serotonin reuptake inhibitor such as sibutramine may be administered with any compound described herein. In still another embodiment, a lipase inhibitor such as orlistat or an inhibitor of lipid absorption such as Xenical may be administered with any compound described herein. The combination of therapeutic agents may act synergistically to decrease body fat or promote weight loss. Using this approach, one may be able to achieve therapeutic efficacy with lower dosages of each agent, thus reducing the potential for adverse side effects.

An additional embodiment of the invention relates to the administration of a composition that generally comprises an active ingredient formulated with a pharmaceutically acceptable excipient. Excipients may include, for example, sugars, starches, celluloses, gums, and proteins. Various formulations are commonly known and are thoroughly discussed in the latest edition of Reminton's Pharmaceutical Sciences (Maack Publishing, Easton Pa.). Such compositions may consist of a Fiaf polypeptide or Fiaf peptidomimetic.

The compositions utilized in this invention may be administered by any number of routes including, but not limited to, oral, intravenous, intramuscular, intra-arterial, intramedullary, intrathecal, intraventricular, pulmonary, transdermal, subcutaneous, intraperitoneal, intranasal, enteral, topical, sublingual, or rectal means.

The actual effective amounts of compound described herein can and will vary according to the specific composition being utilized, the mode of administration and the age, weight and condition of the subject. Dosages for a particular individual subject can be determined by one of ordinary skill in the art using conventional considerations. Those skilled in the art will appreciate that dosages may also be determined with guidance from Goodman & Gilman's The Pharmacological Basis of Therapeutics, Ninth Edition (1996), Appendix II, pp. 1707-1711 and from Goodman & Gilman's The Pharmacological Basis of Therapeutics, Tenth Edition (2001), Appendix II, pp. 475-493.

(D) Methods for Treating Weight-Related Disorders

A further aspect of the invention encompasses the use of the methods to regulate fat storage and weight loss gain in a subject as a means to treat weight-related disorders. In one embodiment, weight-related disorders are treated by modulating the amount of or the activity of Fiaf, as detailed in II(A). In another embodiment, weight-related disorders are treated by altering a subject's gastrointestinal microbial population, as detailed II(B). In still another embodiment, weight-related disorders are treated by administering the combination therapy, as detailed II (C).

In one particularly preferred embodiment, the weight-related disorder is obesity or an obesity-related disorder. A subject in need of treatment for obesity is diagnosed and is then administered any of the treatments detailed herein, such as in sections II (A), (B), or (C). Typically, a subject in need of treatment for obesity will have at least one of three criteria: (i) BMI over 30; (ii) 100 pounds overweight; or (iii) 100% above an “ideal” body weight. In addition, obesity-related disorders that may be treated by the methods of the invention include metabolic syndrome, type II diabetes, hypertension, cardiovascular disease, and nonalcoholic fatty liver disease.

(E) Biomarkers and Screenine for Compounds that Modulate Fiaf Expression or Activity

A further aspect of the invention provides biomarkers that may be utilized in predicting whether a subject is at risk for becoming obese or suffering from an obesity-related condition. In one embodiment, the biomarker is serum Fiaf levels. In a further embodiment, the biomarker is gastrointestinal levels of microbiota that suppress Fiaf transcription.

Yet another aspect of the invention encompasses methods to identify microbial produced compounds that modulate Fiaf transcription or activity and non microbial produced compounds that modulate Fiaf transcription or activity. Generally speaking, methods generally known in the art, such as those described in section I, may be utilized to identify compounds that modulate Fiaf transcription or activity. In one embodiment, a method for screening for a compound that is effective in altering expression of a polynucleotide encoding a Fiaf polypeptide is provided, such as in gnotobiotic zebrafish as shown in Example 10.

In one embodiment, a method for screening for a compound that is effective in altering expression of a polynucleotide (gene) encoding a Fiaf polypeptide is provided. Effective compounds may alter polynucleotide expression by acting on transcriptional or translational regulators of Fiaf expression.

At least one, and up to a plurality, of test compounds may be screened for effectiveness in altering expression of a specific Fiaf polynucleotide. A test compound may be obtained by any method commonly known in the art, including but not limited to selection from an existing, commercially-available or proprietary library of naturally-occurring or non-natural chemical compounds; selection from a library of chemical compounds created combinatorially or randomly, or purification from a natural product, such as extracts of gut microbes grown in vitro or from conditioned medium harvested after culture of a gut microbe or collection of gut microbes. Alterations in the expression of a polynucleotide encoding a Fiaf polypeptide may be assayed by a number of methods commonly known in the art including but not limited to qRT-PCR, as described above. Detection of a change in the expression of a Fiaf polynucleotide, or its protein product, indicates that the test compound is effective in altering Fiaf gene expression. Another embodiment is to observe changes in expression of a transgene containing Fiaf transcriptional regulatory elements responsive to microbial signals, linked to an open reading frame encoding a fluorescent protein reporter, in gnotobiotic zebrafish.

Another embodiment is to test the activity of Fiaf peptides, peptidomimetics or related compounds in germ-free Fiaf-l- mice to determine whether they reduce their high fat content.

Another aspect of the invention encompasses the use of a Fiaf polypeptide to screen for compounds that modulate the activity of the Fiaf polypeptide. Such compounds may include agonists as detailed above. In one embodiment, an assay is performed under conditions permissive for Fiaf polypeptide activity, wherein the Fiaf polypeptide is combined with at least one test compound, and the activity of the subject polypeptide in the presence of a test compound is compared with the activity of the Fiaf polypeptide in the absence of the test compound. Activity could, for example, be defined as the capacity to inhibit LPL-catalyzed biochemical reactions in vitro. A change in the activity of Fiaf in the presence of the test compound is indicative of a compound that modulates the activity of Fiaf polypeptides. At least one and up to a plurality of test compounds may be screened.

In another embodiment, a transgene consisting of transcriptional regulatory elements that are constitutively active in the intestinal epithelium (e.g. nucleotides −1178 to +28 of the rat intestinal fatty acid binding protein gene) linked to Fiaf could be introduced into Fiaf-l-mice so the effects of Fiaf activation can be studied and additional targets for pharmacologic manipulation of Fiaf-related pathways that lead to reduced adiposity can be performed.

A variety of protocols for measuring Fiaf polypeptides, including ELISAs and RIAs, and may be used in any of the screening methods delineated above.

### DEFINITIONS

Acc1 stands for acetyl-CoA carboxylase.

The term “antagonist” refers to a molecule that inhibits or attenuates the biological activity of a Fiaf polypeptide and in particular, the ability of Fiaf to inhibit LPL. Antagonists may include proteins such as antibodies, nucleic acids, carbohydrates, small molecules, or other compounds or compositions that modulate the activity of a Fiaf polypeptide either by directly interacting with the polypeptide or by acting on components of the biological pathway in which Fiaf participates.

The term “agonist” refers to a molecule that enhances or increases the biological activity of a Fiaf polypeptide and in particular, the ability of Fiaf to inhibit LPL. Agonists may include ptoteins, peptides, nucleic acids, carbohydrates, small molecules (e.g., such as metabolites), or other compounds or compositions that modulate the activity of a Fiaf polypeptide either by directly interacting with the polypeptide or by acting on components of the biological pathway in which Fiaf participates.

The term “altering” as used in the phrase “altering the microbiota population” is to be construed in its broadest interpretation to mean a change in the representation of microbes in the gastrointestinal tract of a subject. The change may be a decrease or an increase in the presence of a particular microbial species.

“BMI” as used herein is defined as a human subject's weight (in kilograms) divided by height (in meters) squared.

CHREBP stands for carbohydrate response element binding protein.

CONV-D stands for conventionalization of germ free animals with a gut microbiata harvested from conventionally-raised donor animals.

CONV-R stands for conventionally raised, i.e., aquiring microbes beginning at birth. “Conservative amino acid substitutions” are those substitutions that are predicted to least interfere with the properties of the original protein, i.e., the structure and especially the function of the protein is conserved and not significantly changed by such substitutions.

A “detectable label” refers to a reporter molecule or enzyme that is capable of generating a measurable signal and is covalently or noncovalently joined to a polynucleotide or polypeptide.

An “effective amount” is a therapeutically-effective amount that is intended to qualify the amount of agent that will achieve the goal of a decrease in body fat, or in promoting weight loss. Fas stands for fatty acid synthase.

Fiaf stands for fasting-induced adipocyte factor.

A “gene” is a hereditary unit that has one or more specific effects upon the phenotype of the organism, and that can mutate to various allelic forms.

GF stands for germ free.

LPL stands for lipoprotein lipase.

A “nucleic acid” is a nucleotide polymer of DNA or RNA, it consists of purine or pyrimidine base, e.g. with associated pentose sugars, and phosphate groups.

PPAR stands for peroxisome proliferator-activator receptor.

“Peptide” is defined as a compound formed of two or more amino acids, with an amino acid defined according to standard definitions.

The term “pharmaceutically acceptable” is used adjectivally herein to mean that the modified noun is appropriate for use in a pharmaceutical product; that is the “pharmaceutically acceptable” material is relatively safe and/or non-toxic, though not necessarily providing a separable therapeutic benefit by itself. Pharmaceutically acceptable cations include metallic ions and organic ions. More preferred metallic ions include, but are not limited to appropriate alkali metal salts, alkaline earth metal salts and other physiologically acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include without limitation hydrochloric acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid, oxalacetic acid, fumaric acid, propionic acid, aspartic acid, glutamic acid, benzoic acid, and the like.

A “polypeptide” is a polymer made up of less than 350 amino acids.

“Protein” is defined as a molecule composed of one or more polypeptide chains, each composed of a linear chain of amino acids covalently linked by peptide bonds. Most proteins have a mass between 10 and 100 kilodaltons. A protein is often symbolized by its mass in kDa.

SREBP-1 stands for sterol response element binding protein 1.

“Subject” as used herein typically is a mammalian species. Non-limiting examples of subjects that may be treated by the methods of the invention include a human, a dog, a cat, a cow, a horse, a rabbit, a pig, a sheep, a goat, as well as non-mammalian species including an avian species and a fish species.

A “vector” is a self-replication DNA molecule that transfers a DNA segment to a host cell.

As various changes could be made in the above compounds, products and methods without departing from the scope of the invention, it is intended that all matter contained in the above description and in the examples given below, shall be interpreted as illustrative and not in a limiting sense.

### EXAMPLES

The following examples illustrate the invention.

Part I. Examples 1-4 correspond to section I of the detailed description. EXAMPLE 1

Age-matched groups of 7-15 week-old germ-free NMRI/KI mice were maintained in plastic gnotobiotic isolators on a 12 hour light cycle, and given free access to an autoclaved chow diet (B&K Universal). Males were inoculated with wild-type B. thetaiotaomicron (strain VPI-5482) (L. Hooper, et al. (1999) supra). Mice were sacrificed 10 days later, 2 hours after lights were turned on. The distal 1 cm of the small intestine was used to define the number of colony forming units per ml of extruded luminal contents.

Ileal RNA was isolated from mice with >107 colony forming units (CFU) of bacteria per ml of luminal contents. [Earlier studies had shown that 10 days was sufficient to produce robust colonization of the ileum and that =10⁷ CFU/ml were necessary for full induction of fucosylated glycan production in the ileal epithelium (L. Hooper, et al., (1999) supra; L.Bry, et al., Science 273, 1380 (1996))].

Total ileal RNA samples were prepared from the 3 cm of intestine adjacent the distal 1 cm of the small intestine of 4 mice from 3 independent colonizations, and from age- and gender-matched germ-free mice (n=8), using a RNA (Qiagen RNeasy kit). Ileal RNAs from each treatment group were pooled, in equal amounts, for generation of biotinylated cRNA targets. Two targets were prepared, independently, from 30 μg of each total cellular RNA pool, using the method outlined by C. K. Lee, et al., Science 285, 1390 (1999)).

SYBR green-based real-time quantitative RT-PCR studies (N. Steuerwald, et al., Mol. Hum. Reprod., 5, 1034 (1999)) were performed using the gene-specific primers listed in Table 3 above and DNAse-treated RNAs. Control experiments established that the signal for each amplicon was derived from cDNA and not from primer dimers or genomic DNA. Signals were normalized to an internal reference mRNA (glyceraldehyde 3-phosphate dehydrogenase). The normalized data were used to quantitate the levels of a given mRNA in germ-free and colonized ileums (AACT analysis; Bulletin #2, ABI Prism 7700 Sequence Detection System).

Each cRNA was hybridized to Affymetrix Mu11K and Mu19K chip sets representing about −25,000 unique mouse genes from Unigene Build 4 and the TIGR cluster databases, according to Affymetrix protocols. Data collected from each chip were scaled so that the overall fluorescence intensity across each chip was equivalent (target intensity =150). Pairwise comparisons of ‘germ-free’ versus ‘colonized’ expression levels were performed.

A 2-fold or more difference was recorded if three criteria were met: the GeneChip software returned a difference call of “increased” or “decreased,” the mRNA was called ‘present’ by GeneChip software in either germ-free or colonized cRNA, and the difference was observed in duplicate microarray hybridizations.

mRNAs represented by 118 probe sets changed by at least 2-fold with colonization, as defined by duplicate microarray hybridizations.

It was found that transcripts represented by 95 probe-sets were increased, while those lo represented by 23 probe-sets were decreased. The genes represented by 84 of these probe sets (71 unique genes) were assigned to fumctional groups and these are set out in Table 1. In this table, results are presented as the fold-difference in mRNA levels between colonized and germ-free ileum and represent average values from duplicate microarray hybridizations. The average fold-changes for genes represented by 2 or more independent probe sets are listed separately.

Importantly, a large of number of the genes identified using these criteria are involved in modulating fundamental intestinal functions: 20 of the 71 genes (28%) were grouped under nutrient uptake and metabolism. There was also a concerted rise in expression of several components of the host's lipid absorption/export machinery, including pancreatic lipase-related protein-2 (PLRP-2), colipase, liver fatty acid binding protein (L-FABP), and apolipoprotein A-IV (Table 1). As noted above, there was a prominent decrease in expression of Fiaf, a novel PPARy target known to be induced with fasting (S. Kersten, et al., J. Biol. Chem. 275. 28488 (2000)).

Additionally, there were changes in expression of four genes involved in dietary metal absorption. A high affinity epithelial copper transporter (CRTI) mRNA was increased, while metallothionein-I, metallothionein-II, and ferritin heavy chain mRNAs were decreased (Table 1). These changes suggest that colonization engenders increased capacity to absorb heavy metals (e.g., via CRT1) and a concomitant decreased capacity to sequester them within cells (MT-I/II, ferritin). This implies greater host demand for these compounds, either due to increased utilization by the host's own metabolic pathways or to competition with the microbe. The changes in SGLT-1, colipase, L-FABP, and MTI (plus 8 other mRNAs discussed below), were independently validated by qRT-PCR (C.A. Heid, et al., Genome Res., 6, 986 (1996) (Table 2).

Of these, genes which were found to have a difference in expression levels of 5-fold or more as a result of B. thetaiotaomicron colonization were colipase, liver fatty acid binding protein, fasting-induced adipose factor, metallothionein I and metallothionein II, malate oxidoreductase, Sprr2a, angiogenin-4, angiogenin-related protein, gelsolin, gp106(TB2/DP1) and rac 2. Of these, colipase, Fiaf, angiogenin-4 and Sprr2a genes showed a difference in expression levels of 9-fold or more.

A notable feature of the host response to B. thetaiotaomicron was the absence of detectable or changed expression of the many genes involved in immuno-inflammatory processes that are represented on the microarrays. These include genes involved in the NF-κB-regulated processes that are critical regulators of host responses to invasive pathogens (D. Elewaut, et al., J. Immunol. 163, 1457 (1999)). The absence of these responses can be contrasted to results obtained in a recent cDNA microarray analysis of the response of a human intestinal epithelial cell line to Salmonella, an invasive gut pathogen (L. Eckmann, et al. , J. Biol. Chem. 275. 14084 (2000)). The lack of evidence for an evoked in vivo immuno-inflammatory response is consistent with the host's need to accommodate resident gut microbes, such as B. thetaiotaomicron, for its entire lifespan.

### EXAMPLE 2

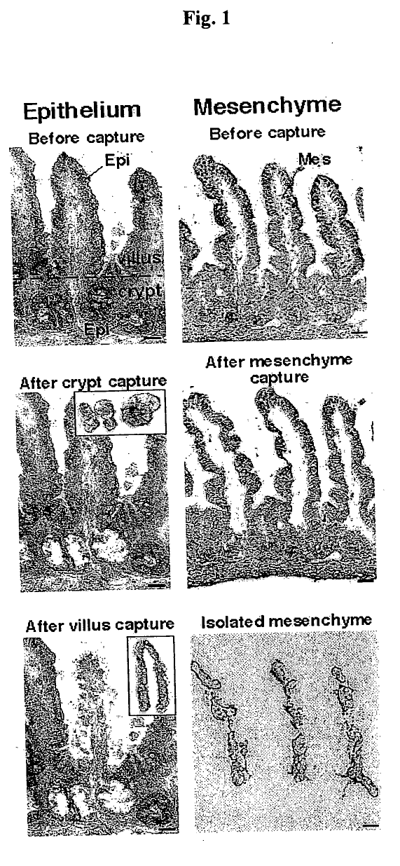

In a further analysis two techniques were combined. First, laser-capture microdissection (LCM) was used to recover three cell populations from frozen sections of ileum harvested immediately after sacrifice of germ-free and colonized mice. The three populations are (i) epithelium present in crypts (the proliferative compartment of the intestine containing undifferentiated cells as well as differentiated members of the Paneth cell lineage); (ii) epithelium overlying villi (containing post-mitotic, differentiated members of the intestine's other three lineages); and (iii) mesenchyme underlying crypt-villus units (FIG. 1).

LCM was performed on groups of mice independent of those used to generate RNA for the microarray analysis. 7 μm-thick sections were cut from frozen ileums and LCM conducted using the PixCell II system from Arcturus (7.5 μm diameter laser spot). RNA was prepared from dissected cell populations using the RNA Micro-Isolation Kit (Strategene) and standard histochemical protocols. (LCM was carried out using conventional methods as described by M. R. Emmert-Buck, et al., Science, 274, 998 (1996) and R. F. Bonner, et al., Science, 278, 1203 (1997).)

The results are shown in FIG. 1.

Second, real-time RT-PCR was used to quantitate levels of specific mRNAs in the laser captured cell populations. The LCM/qRT-PCR analysis was performed using germ-free and colonized mice from three experiments that were independent of those used for microarray profiling.

Each sample was analyzed in triplicate in four-independent experiments. Mean values for the independent determinations ±1 S. D. are shown in Table 2.

Therefore, LCM and real-time RT-PCR analysis were employed to delineate the cellular origins of its response to B. thetaiotaomicron.

The results show that Sprr2a mRNA is confined to the epithelium where its concentration is 7-fold higher on the villus compared to the crypt (FIG. 1B). B. thetaiotaomicron elicits a 280-fold increase in the villus epithelium. This value is in good agreement with the increase documented in total ileal RNA (Table 2). The cellular origin of the Sprr2a response supports the hypothesis that it participates in fortifying the intestinal epithelial barrier in response to bacterial colonization.

Colipase is produced by the exocrine acinar cells of the pancreas. Expression in the intestine had not been reported previously. LCM/qRT-PCR revealed that colipase mRNA is also present in the ileal crypt epithelium, where it increases 10-fold upon B. thetaiotaomicron colonization (FIG. 1B). This accounts for the increase detected by microarray and qRT-PCR analyses of total ileal RNA (Tables 1, 2). Colipase plays a critical role in dietary lipid metabolism by stimulating the activity of both pancreatic triglyceride lipase and PLRP-2 (M. E. Lowe, etal., J. Biol. Chem. 273, 31215 (1998)).

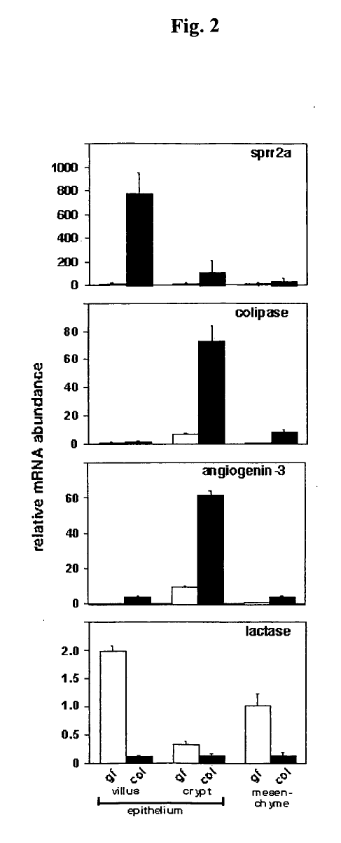

LCM and qRT-PCR revealed that the crypt epithelium is the predominant location of a gene, amplifiable using primers such as SEQ ID NO 12 and 25 (see Table 3 hereinbefore), which encodes a new protein, angiogenin-4 (see example 4 below). However, LCM and real-time RT-PCR analysis revealed that in colonized ileum, the levels of this mRNA are highest in crypt epithelium (values in the ileal villus epithelium and mesenchyme are 14- and 15-fold lower, respectively; FIG. 2).

The LCM/qRT-PCR studies of Sprr2a colipase and angiogenin-4 establish the feasibility of assigning an in vivo host response to a particular cell population in a complex tissue, and of describing the cellular response in quantitative terms. In recovering a responding cell population and expressing its reaction to a microorganism in quantitative terms, the applicants' results demonstrate how it is possible to move beyond in vitro models and use in vivo systems to study the impact of a microbe on host cell gene expression.

Colonization of germ-free mice with B. thetaiotaomicron produces a decrease in ileal LPH mRNA levels (Table 1, 2). Analysis of RNA isolated from laser-captured epithelial and mesenchymal cell populations established that the colonization-induced reduction in LPH mRNA levels occurs primarily within the villus epithelium (FIG. 2).

Comparison of transcript levels between germ-free and B. thetaiotaomicron-associated mice revealed a colonization-associated increase in expression of angiogenin-4.

### EXAMPLE 3

The concept that microbes such as B. thetaiotaomicron may help legislate changes in expression of a given gene in the intestine, raises the question of whether some or many components of the microbiota can elicit these changes.

In order to examine this, age-matched groups (n=4-8 mice/group) of 7-15 week-old germ-free NMR1/KI mice were maintained in plastic gnotobiotic isolators on a 12 hour light cycle, and given free access to an autoclaved chow diet (B&K Universal). Males were inoculated with one of the following groups.

(i) Nothing--Germ-free control,

(ii) B. thetaiotaomicron strain VPI-5482 (L. V. Hooper, et al., Proc. Natl, Acad. Sci. U.S.A. 96.9833 (1999)).

(iii) E. coli K12 which was originally recovered from a normal human fecal flora,

(iv) Bifidobacterium infantis (ATCC 15697), a prominent component of the pre-weaning human and mouse ileal flora and a commonly used probiotic.

(v) a ‘complete’ ileal/cecal microbiota harvested from conventionally-raised mice (L. Bry, et al., Science 273, 1380 (1996)).

A further control group comprised mice conventionally raised since birth.

Mice were sacrificed 10 days later, 2 hours after lights were turned on. The distal 1 cm of the small intestine was used to define CFU/ml ileal contents. The 3 cm of intestine just proximal to this segment was used to isolate total ileal RNA (Qiagen RNeasy kit).

qRT-PCR was used to compare ileal lactase mRNA levels in each group (all animals had=10⁷ CFU/ml ileal contents). The results are shown in FIG. 3.

Colonization with any of the three gram-negative anerobes elicited an equivalent decline in lactase expression relative to germ-free controls (FIG. 3). This decline was also observed after inoculation of a complete ileal/cecal flora. qRT-PCR of the same RNAs revealed that ileal expression of colipase and angiogenin-4 was induced after colonization of all three organisms, and by the ileal/cecal flora (FIG. 3).

The levels of colipase and angiogenin-4 mRNAs achieved in the ileums of these ex-germ-free mice were comparable to those of age-matched mice that have been conventionally-raised since birth (FIG. 3).

In contrast to these findings, the response of sprr2a to colonization was dependent upon the colonizing species. While B. thetaiotaomicron produced a pronounced rise in Sprr2a mRNA that recapitulates the response to a 10 day colonization with the ileal/cecal flora, colonization with B. infantis and E. coli produce only negligible increases in mRNA levels (FIG. 3).

Mdrla and glutathione-S-transferase, which act in concert to metabolize xenobiotics and electrophiles, also exhibited species-specific (and concerted) responses. Unlike B. thetaiotaomicron, which suppresses expression, E. coli and B. infantis both elicit increases in these mRNAs. In contrast, the multi-component ileal/cecal flora did not produce a significant (i.e., =2-fold) change in levels of either mRNA when compared to germ-free controls,

The Mdr1 a/GST responses provide direct evidence that components of the normal microflora can modulate host genes involved in drug metabolism, and suggest that variations in drug metabolism between individuals may arise, in part, from differences in their resident gut microbiota.

### EXAMPLE 4

Following the observation that a 10 d colonization was associated with a 11-fold increase in ileal expression of a mRNA detected by an Affymetrix-designed probe-set designed from the published sequence of angiogenin-3, we designed primers specific for the 3′ and 5′ ends of the mouse angiogenin-3. They were:

ORF forward primer:

(SEQ ID NO 27)

5′-CCTTGGATCCATGGTGATGAGCCCAGGTTCTTTG

which incorporates a BamHI site at the 5′ end;

reverse primer:

(SEQ ID NO 28)

5′-CCTTTCTAGACTACGGACTGATAAAAGACTCATCGAAG

which incorporates an XbaI site at the 5′ end.

These primers were used together with RT-PCR to amplify a 438 bp sequence from RNA prepared from the ileums of ex-germ-free NMRI mice. These mice had been colonized for 10 d with a complete ileal/cecal flora harvested from conventionally-raised animals belonging to the same inbred strain. We subcloned the PCR product into BamHI/XbaI digested pGEX-KG and sequenced it using vector-specific primers.

Surprisingly, the nucleotide sequence of the ORF was only 90% identical to that of mouse angiogenin-3. Since the primer sequences used in the PCR reaction (specific for angiogenin-3) were incorporated into the product, we used 5′- and 3′-RACE to (a) obtain accurate sequence at the 5′ and 3′ ends of the ORF of this new angiogenin, and (b) characterize the 5′- and 3′ untranslated regions of its mRNA. The results revealed only 88.3% nucleotide sequence identity with angiogenin-3 mRNA.

The nucleotide sequence that encodes the angiogenin-4 protein, aligned with the angiogenin-3 sequence is shown hereinafter in FIG. 4 as SEQ ID NO 29 and 30, respectively.

Angiogenin-4 has 74 to 81% amino acid sequence identity to the other 3 members of the mouse angiogenin family (FIG. 5). It was found that the 5′ and 3′-untranslated regions of angiogenin-4 are closely related to the corresponding regions of angiogenin-3 mRNA (FIG. 4).

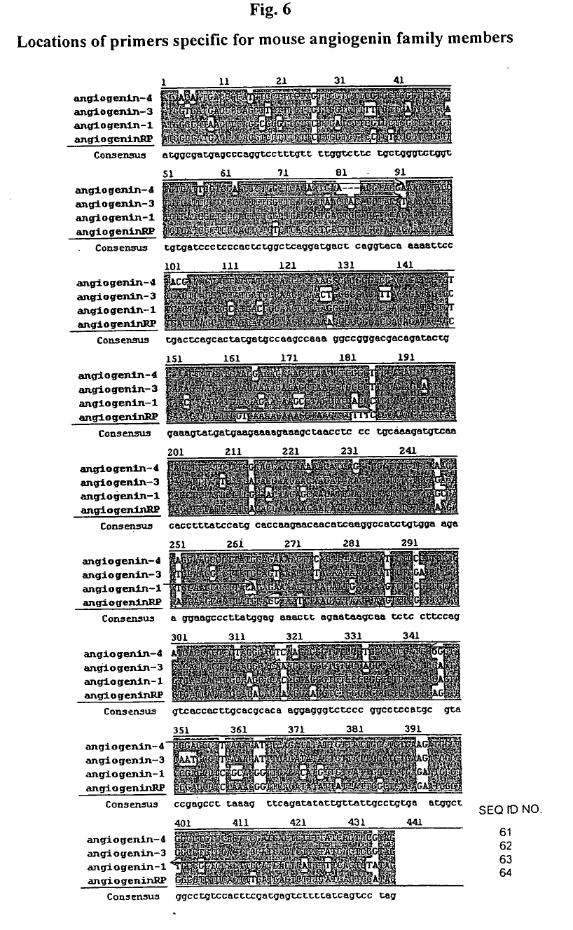

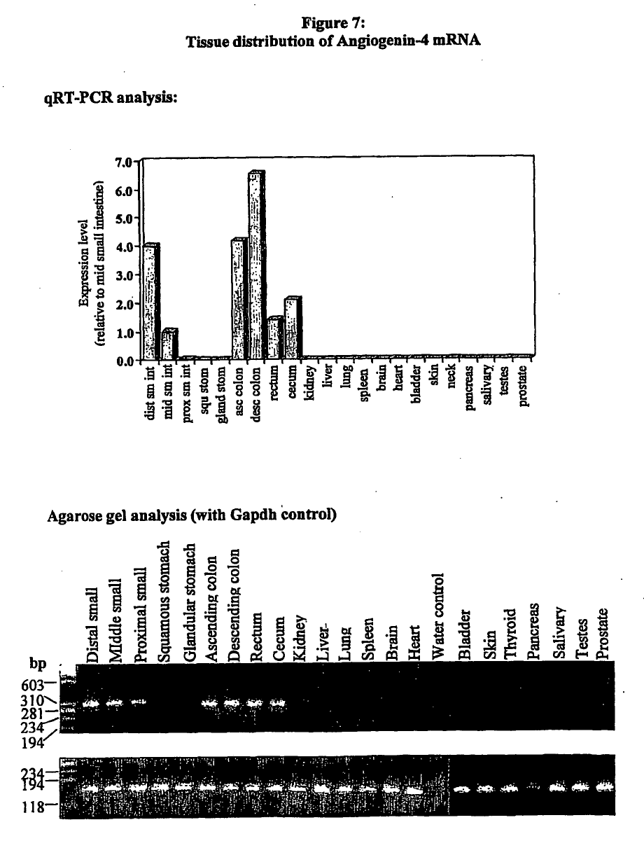

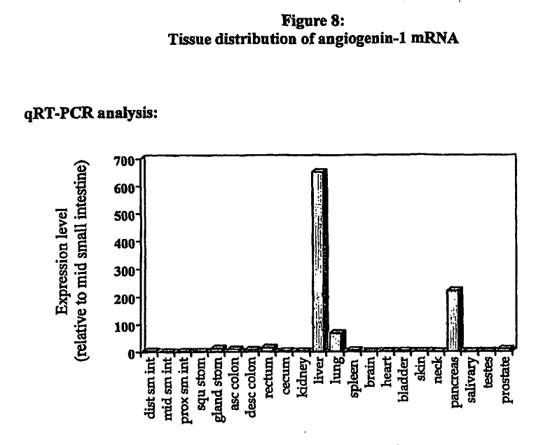

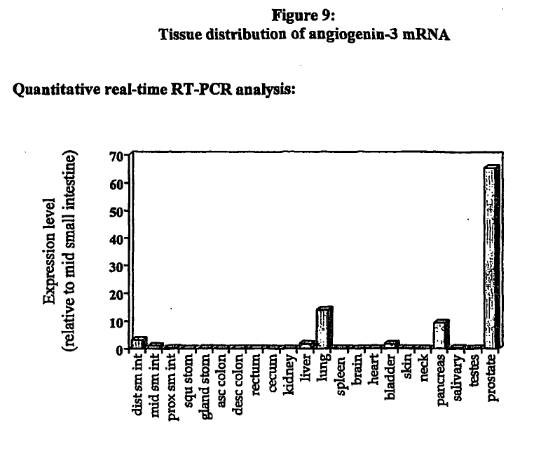

Subsequently a comparative analysis of the tissue distribution of the various mouse angiogenin mRNAs, was conducted. cDNA was synthesized from RNAs isolated from tissues harvested from conventionally raised adult (12-14 week old) male and female NMRI mice (25 tissues/mouse). To quantitate relative levels of expression of each gene, we designed primer sets specific for each of the four mouse angiogenin family members (FIG. 6; Table 4 below) and used them for SYBR-Green-based real-time quantitative RT-PCR (qRT-PCR) analyses.

TABLE 4

| | | SEQ ID | |
|---|---|---|---|
| Gene | Primer | NO. | Sequence |
| angiogenin-4 | forward | 35 | 5′ CTCTGGCTCAGAATGTAAGGTACGA |
| | reverse | 36 | 5′ GAAATCTTTAAAGGCTCGGTACCC |
| angiogenin-3 | forward | 37 | 5′ CTGGCTCAGGATAACTACAGGTACAT |
| | reverse | 38 | 5′ GCCTGGGAGACCCTCCTTT |
| angiogenin-1 | forward | 39 | 5′ AGCGAATGGAAGCCCTTACA |
| | reverse | 40 | 5′ CTCATCGAAGTGGACCGGCA |
| angiogenin | forward | 41 | 5′ GGTGAAAAGAAAGCTAACCTCTTTC |
| related protein | reverse | 42 | 5′ AGACTTGCTTATTCTTAAATTTCG |

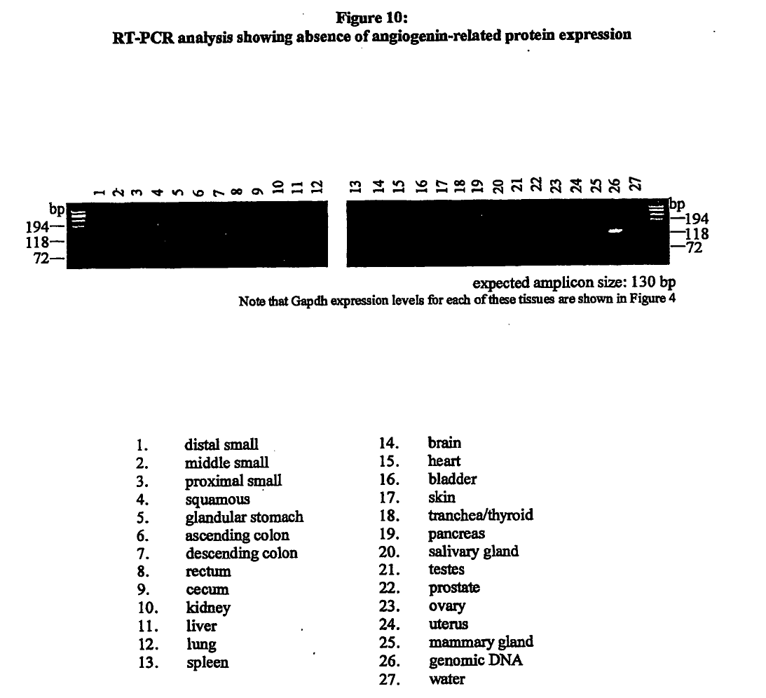

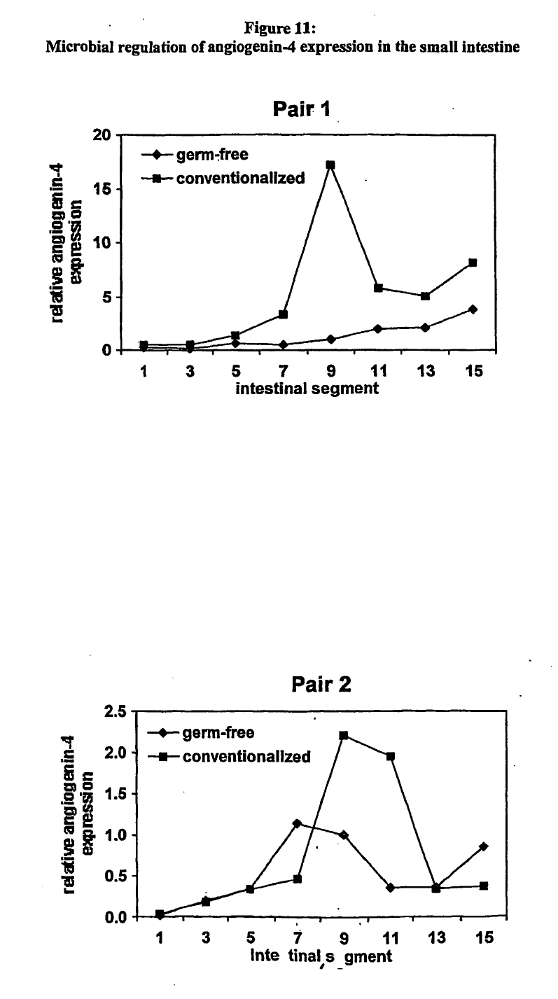

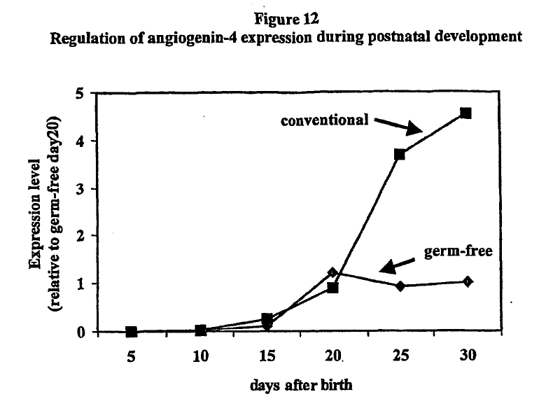

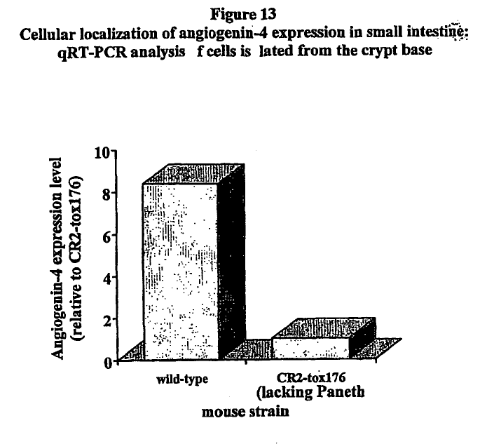

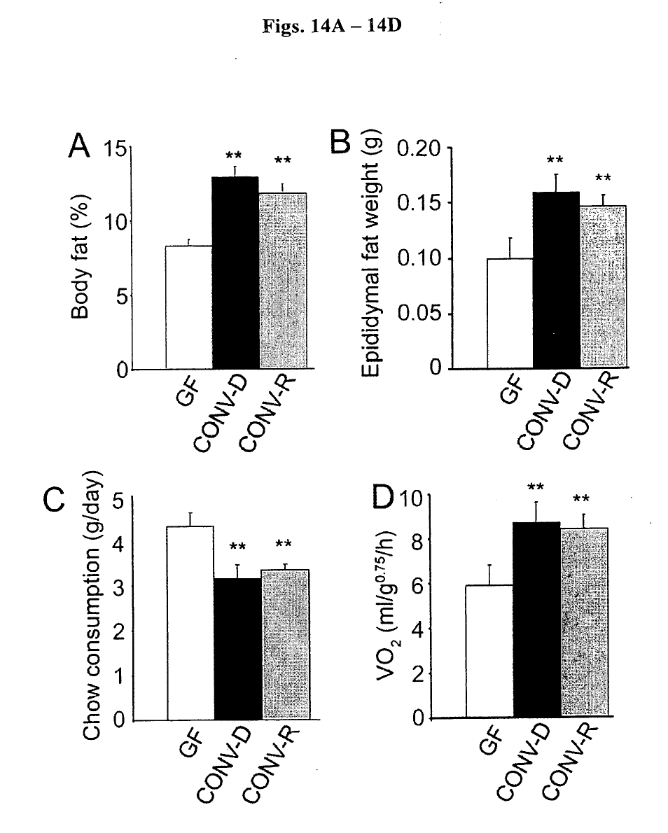

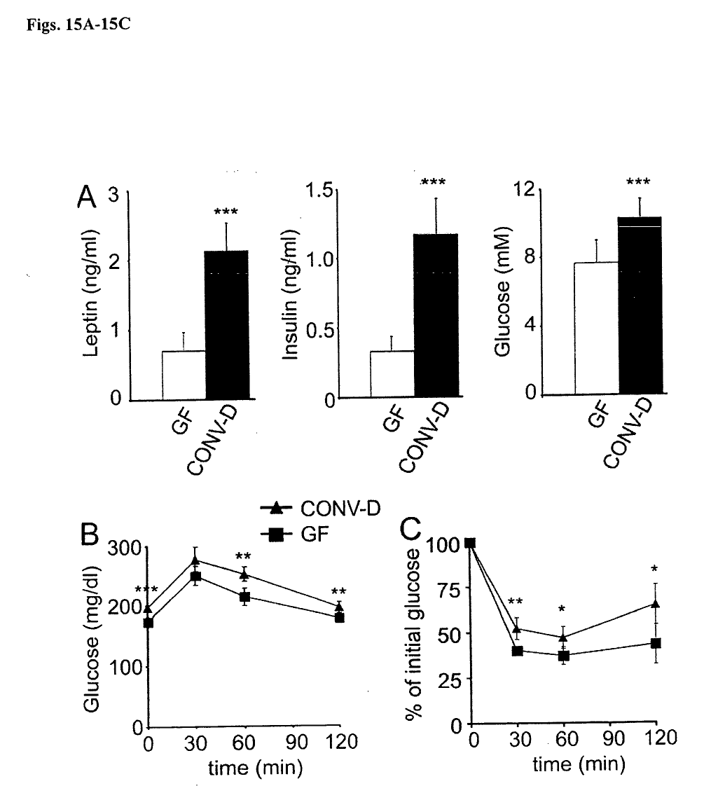

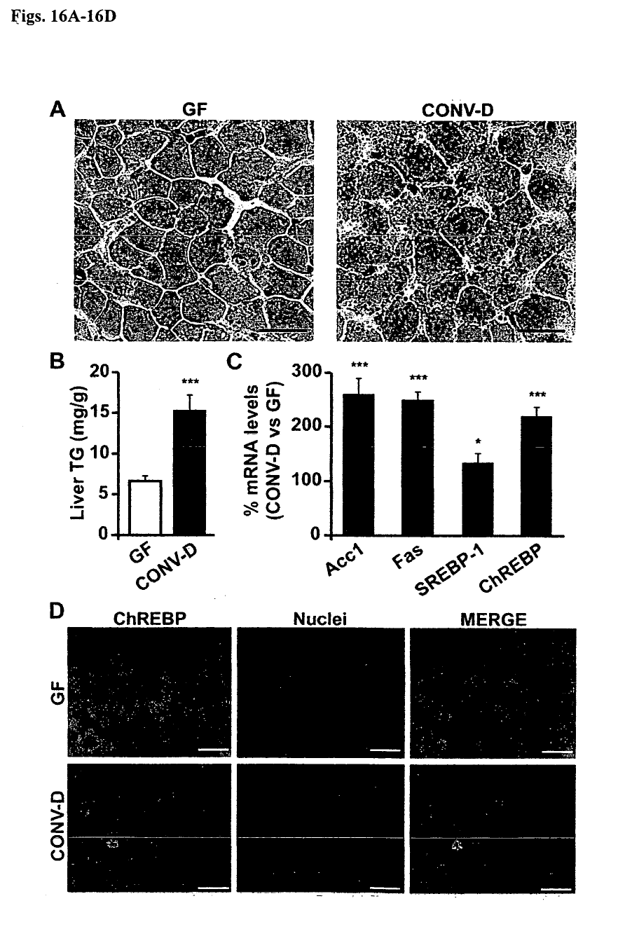

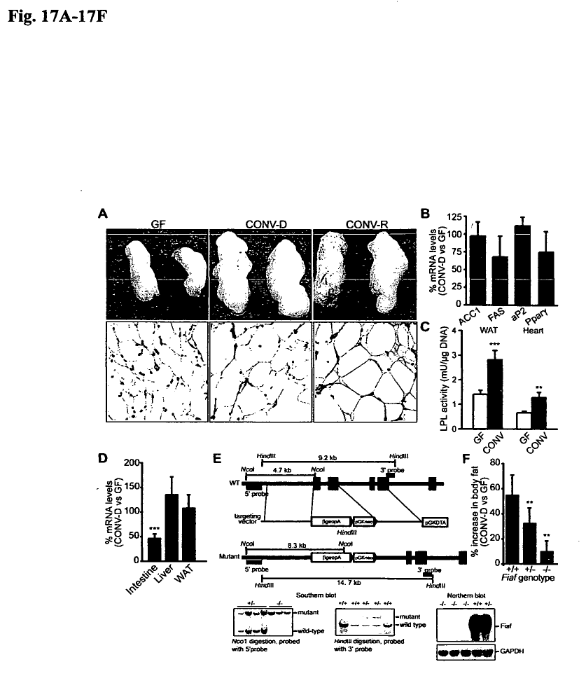

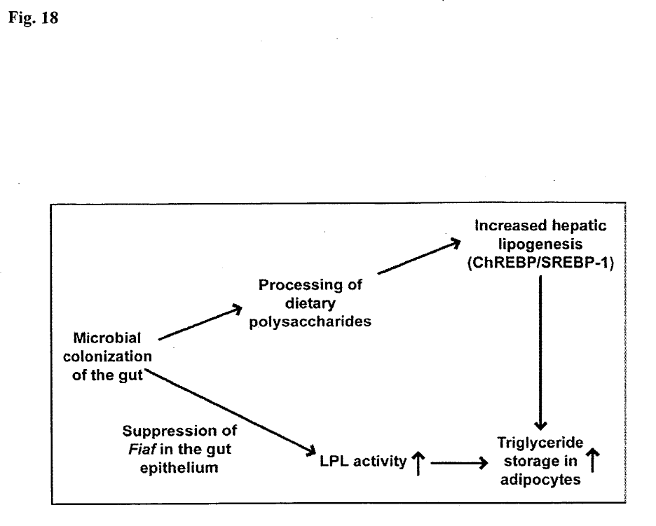

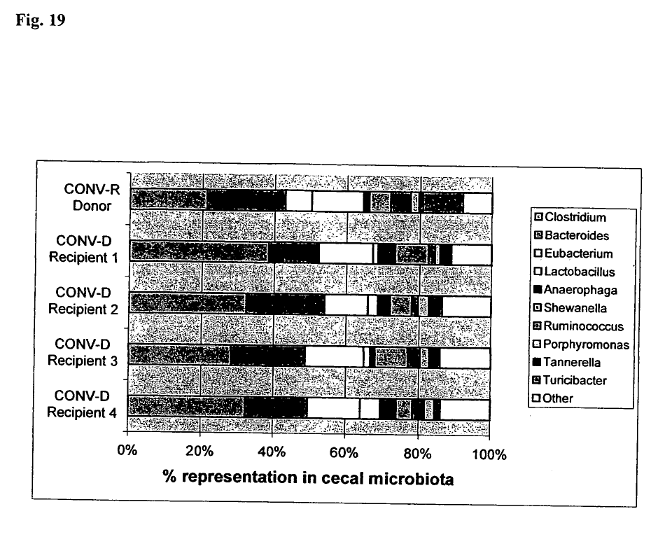

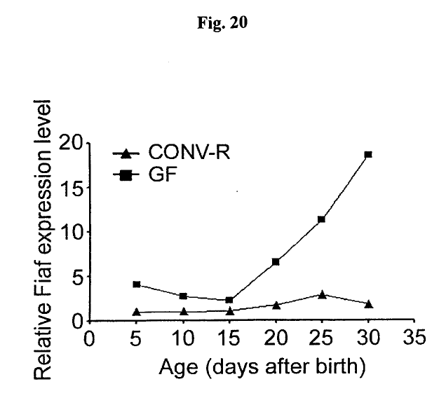

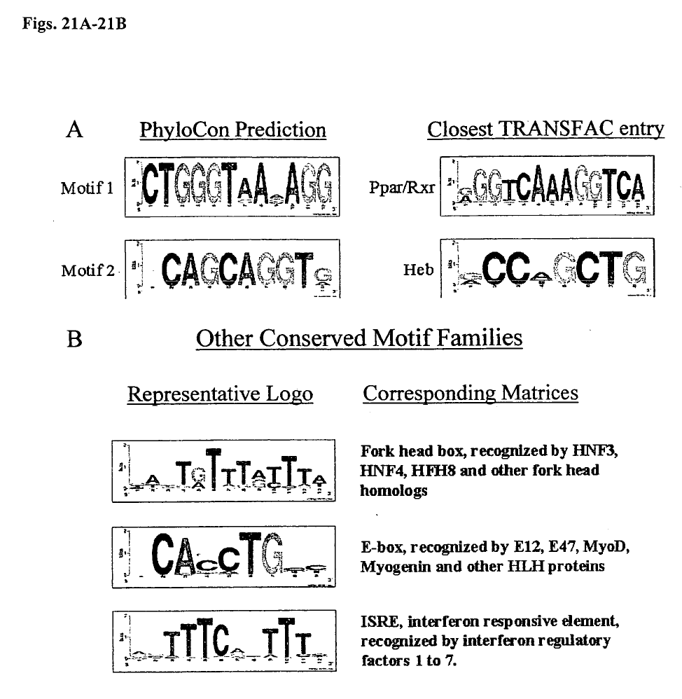

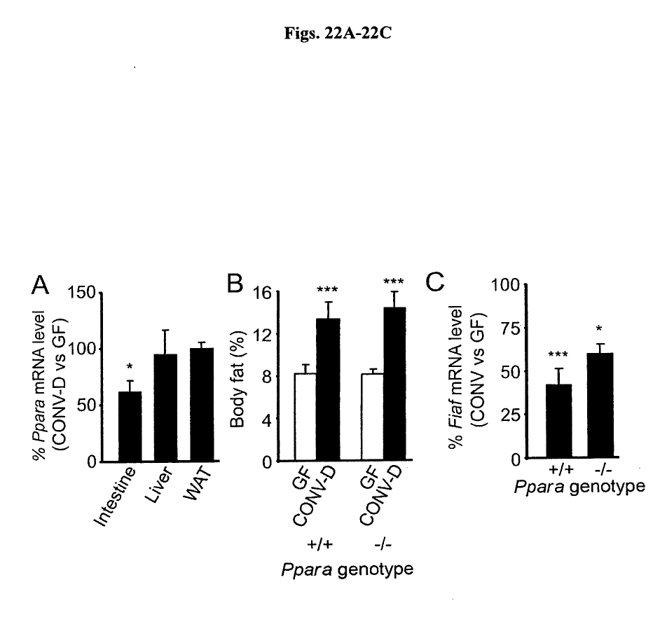

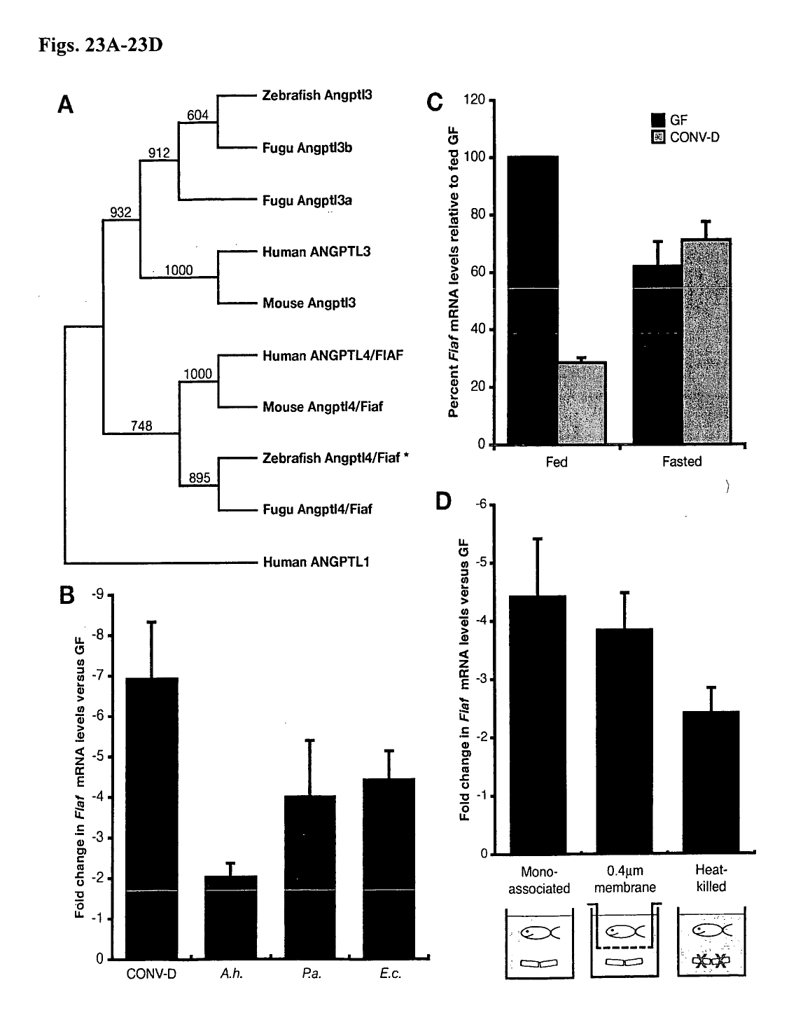

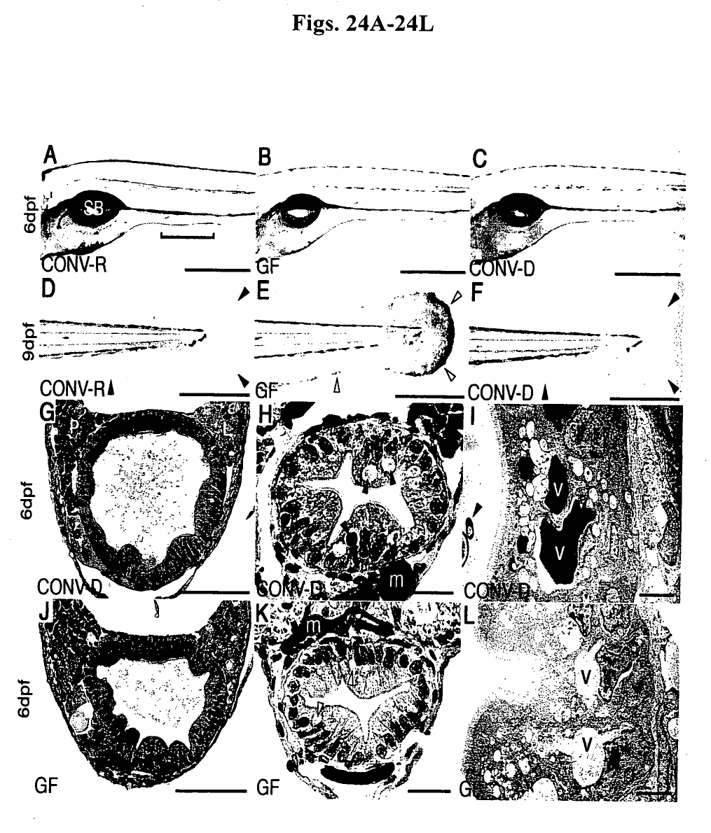

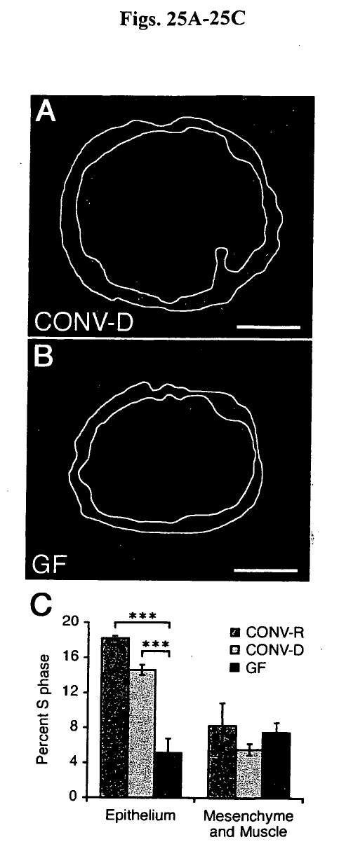

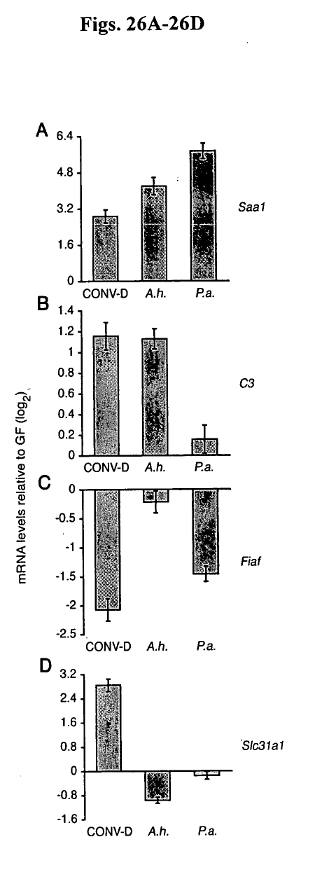

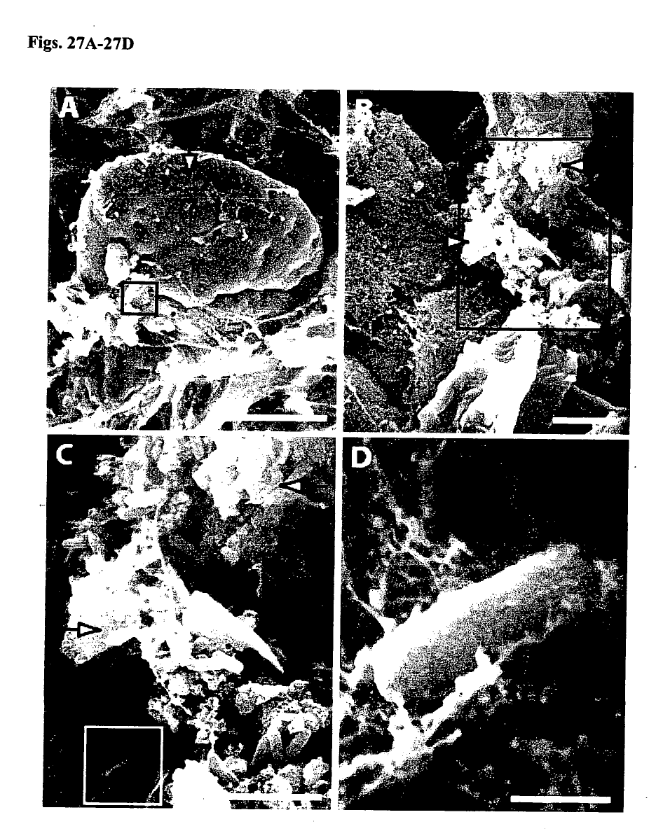

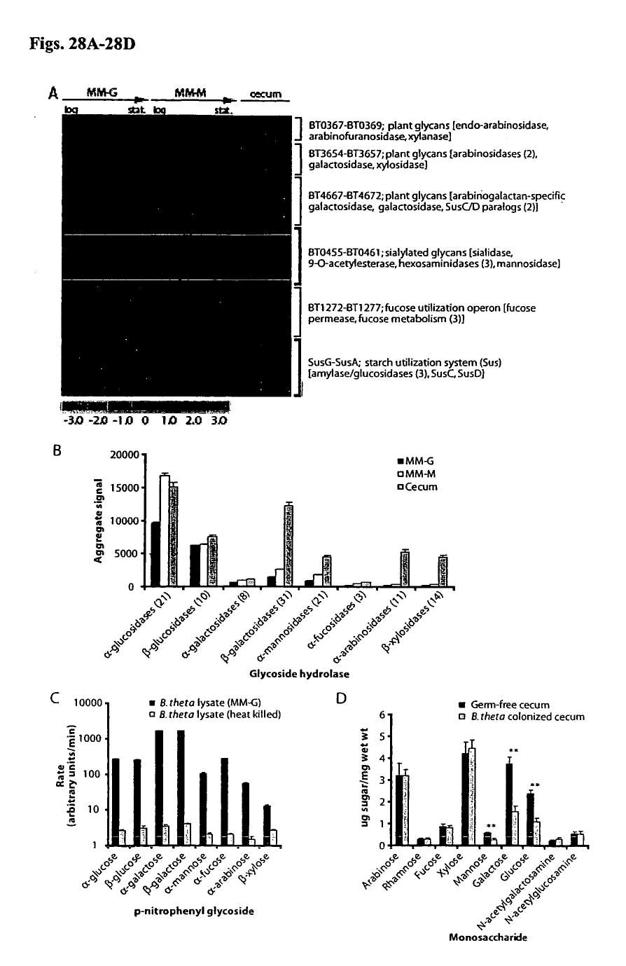

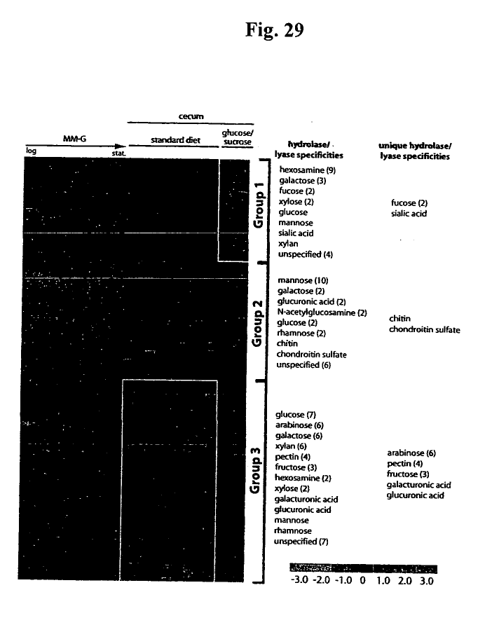

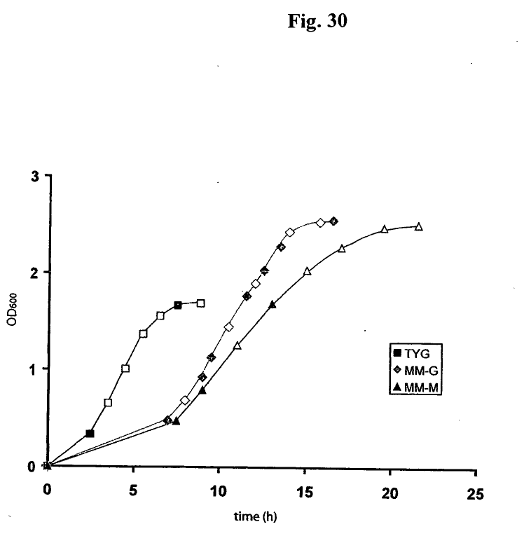

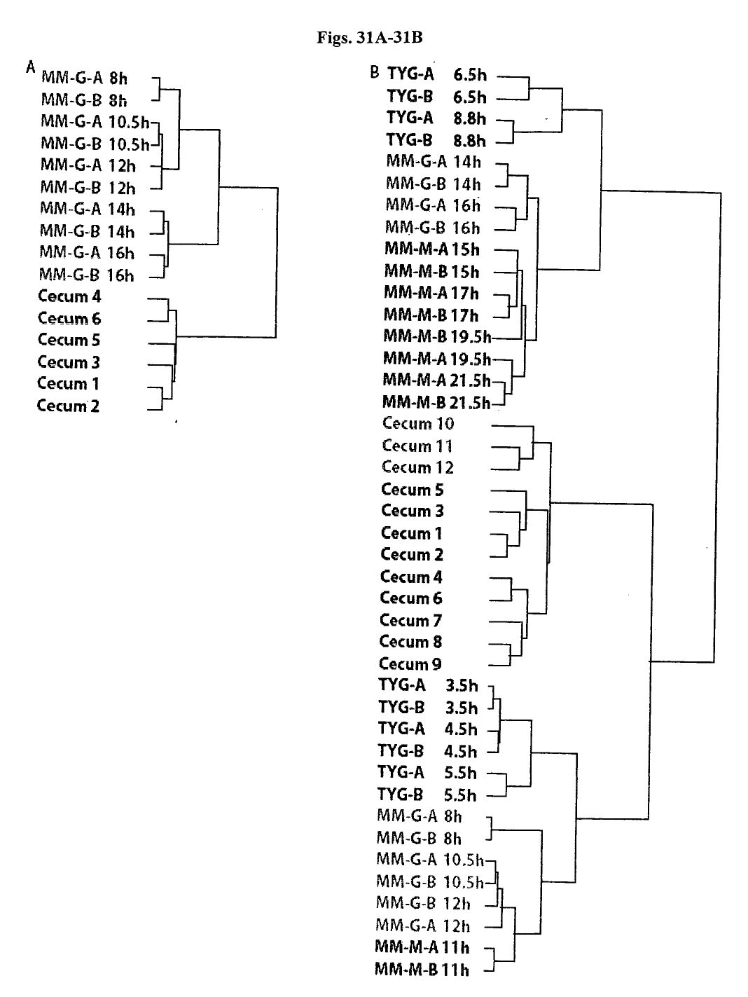

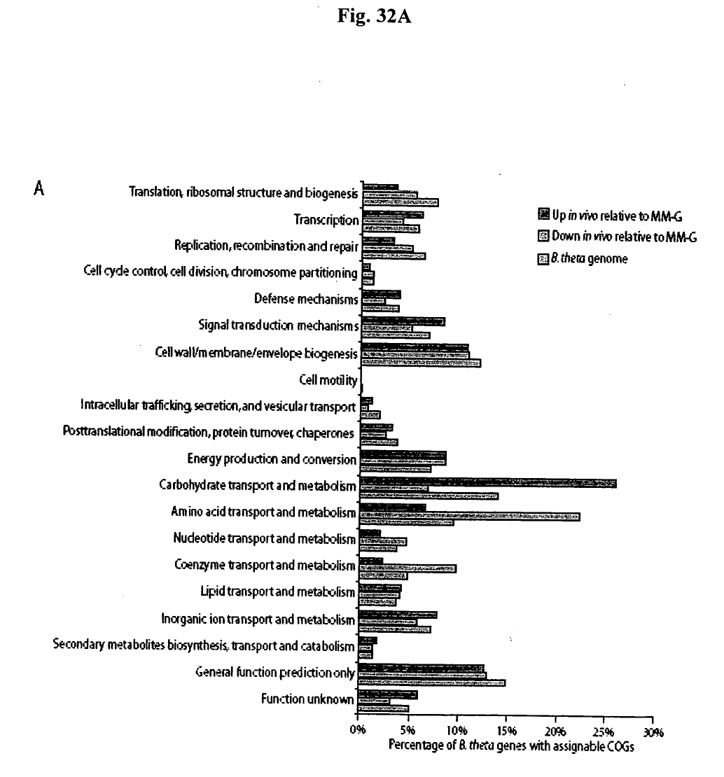

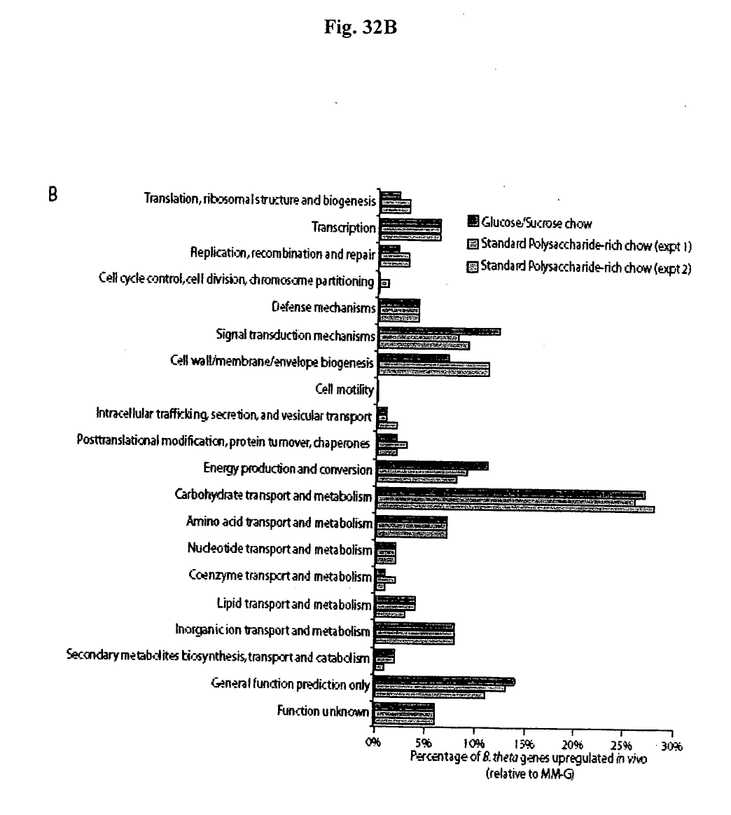

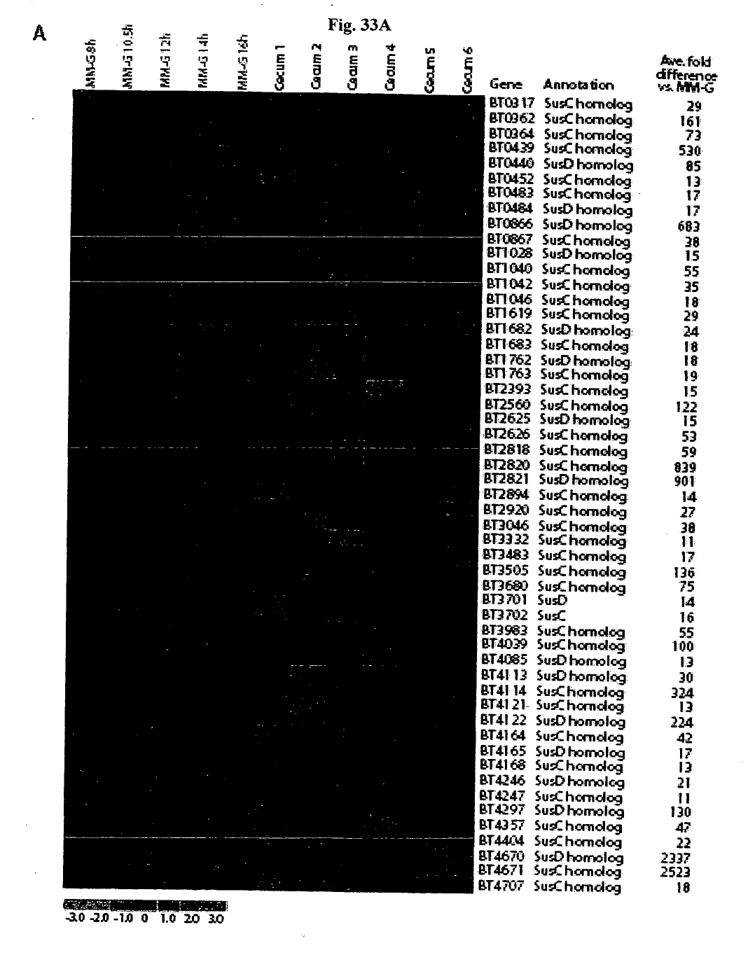

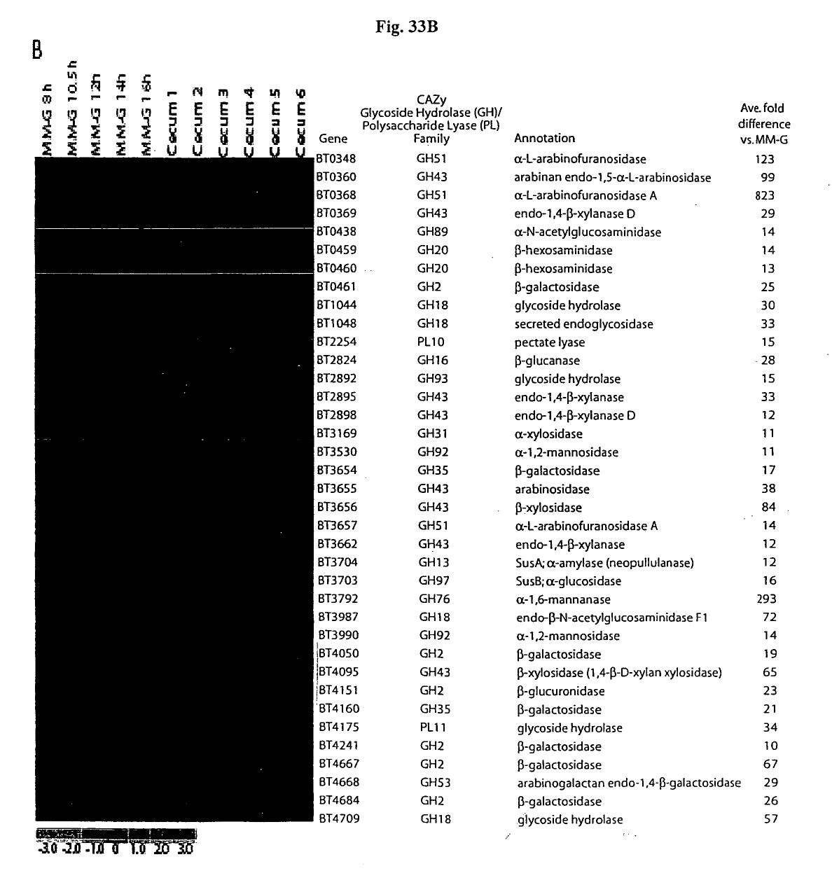

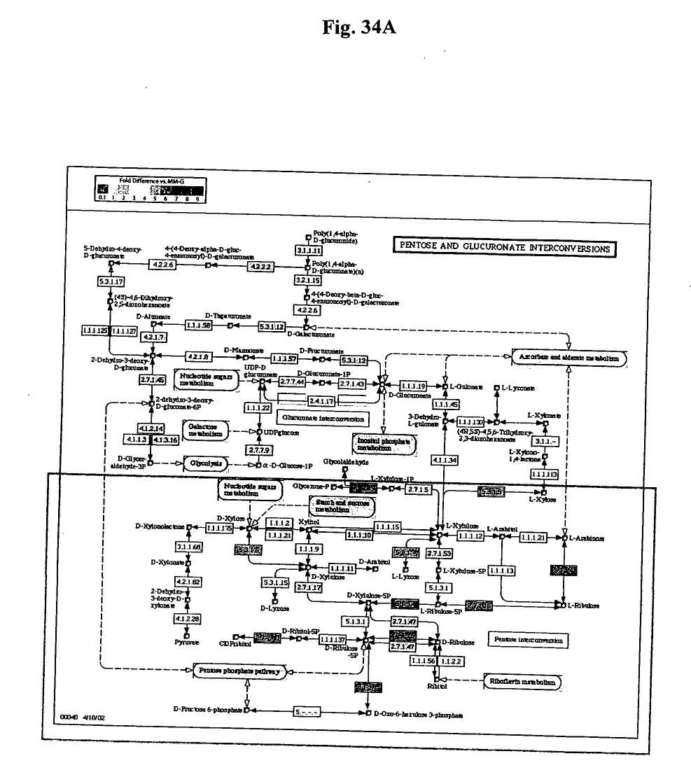

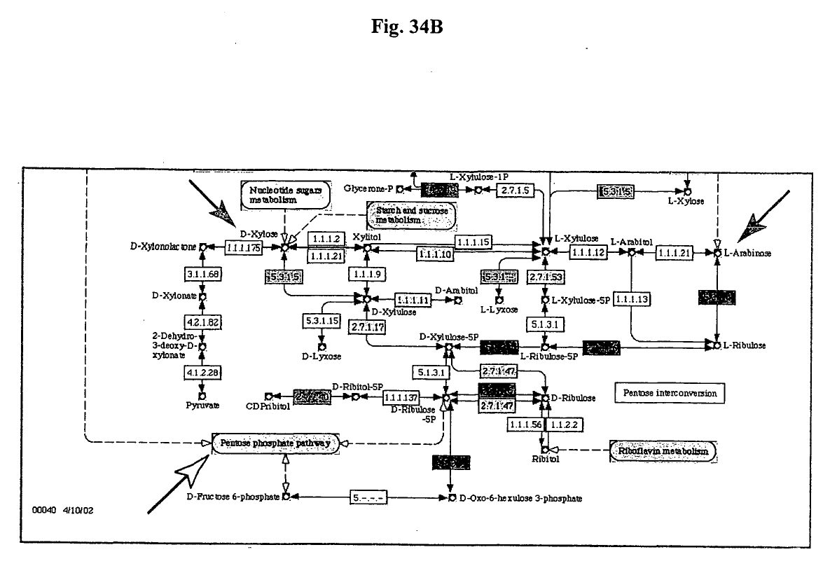

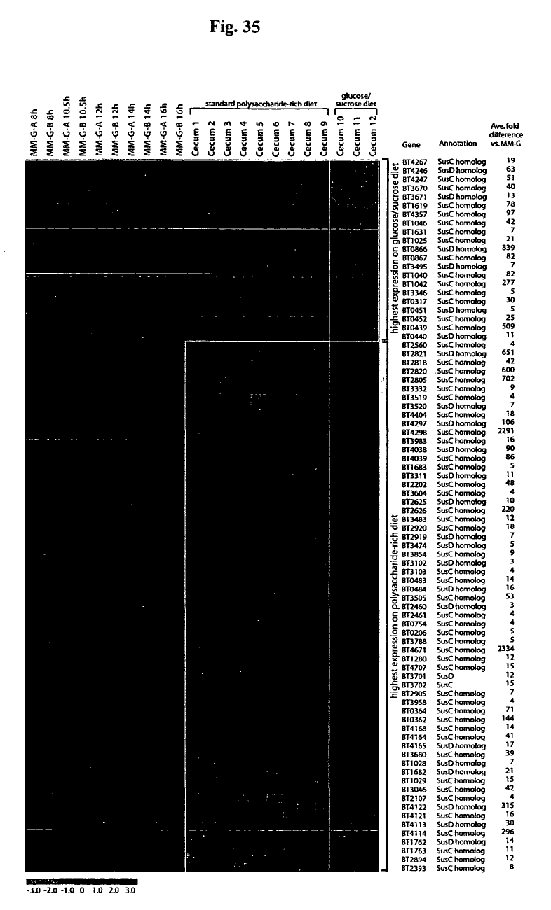

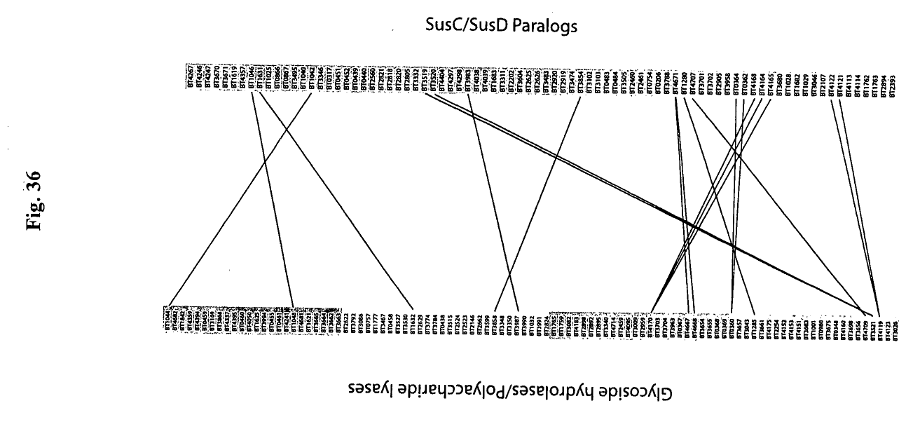

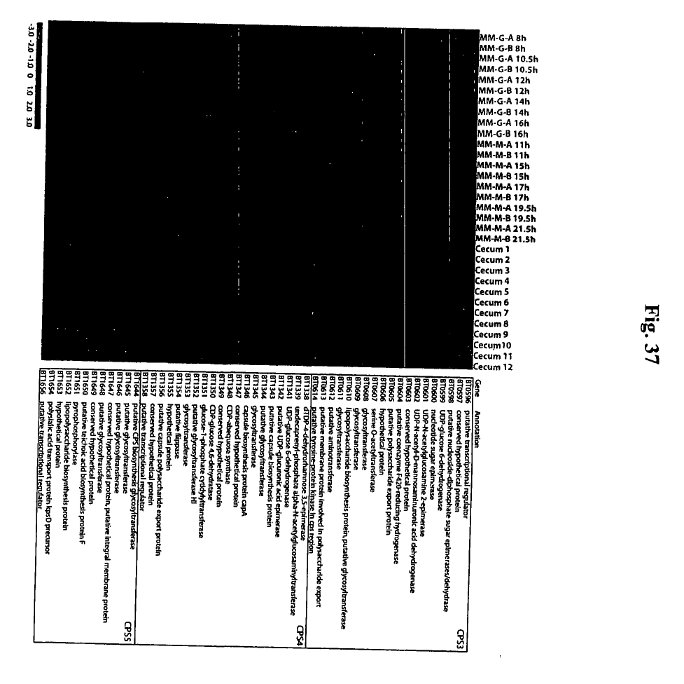

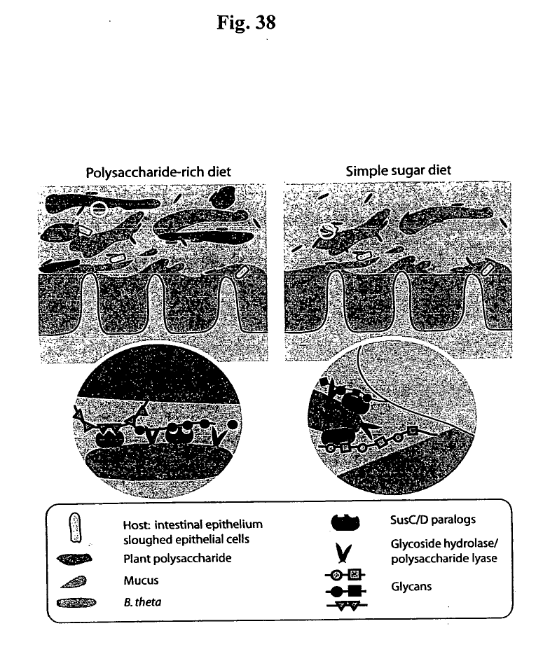

Remarkably, angiogenin-4 mRNA was restricted the intestine where it is expressed from the duodenum to the rectum (FIG. 7). In contrast, angiogenin-1 expression is highest in liver, lung, and pancreas (FIG. 8), while angiogenin-3 is expressed primarily in liver, lung, pancreas, and prostate (FIG. 9). Angiogenin-related protein mRNA was undetectable in all tissues surveyed even after 40 cycles of PCR (FIG. 10).

Thus, the highly restricted, intestine-specific pattern of angiogenin-4 expression makes it unique among mouse angiogenin family members.

These findings indicated that there was microbial-regulation of angiogenin-4 rather than angiogenin-3 expression in the intestine. To test this hypothesis directly, angiogenin-4-specific primers and qRT-PCR were used to compare angiogenin-4 mRNA levels along the length of the small intestine of germ-free NMRI mice and germ-free mice colonized for 10 d with an ileal/cecal flora harvested from conventionally raised NMRI animals. Pair-wise comparisons revealed that expression of angiogenin-4 is highest in the jejunum of colonized mice, and that conventionalization induces up to a 17-fold increase in angiogenin-4 expression in this region (FIG. 11). Mono-association of germ-free NMRI mice with B. thetaiotaomicron for 10 d resulted in a comparable induction of angiogenin-4 expression (data not shown). Regulation of Angiogenin-4 Expression During Postnatal Development is Consistent with its Microbial Regulation

The developmental patterns of angiogenin-4 expression in postnatal day 5 (P5) to P30 germ-free and conventionally raised NMRI mice (n=3 mice per time point per group) was then assessed (FIG. 9). Relative levels of the angiogenin-4 transcript remained relatively low until P20 in both groups of mice. Expression rose slightly (2-3 fold) in germ-free animals after this time point. In contrast, angiogenin-4 expression increased more than 20-fold between P15 and P30 in conventionally-raised animals. These results indicate that angiogenin-4 is induced during the suckling/weaning transition -coincident with a major shift in the gut microbiota. The lack of angiogenin-4 induction in postnatal germ-free mice is also consistent with the conclusion that components of the microbiota play an important role in regulating angiogenin-4 expression.

Cellular Localization of Angiogenin-4

The previous laser capture microdissection (LCM)/qRT-PCR study of the cellular origins of angiogenin protein expression (Example 2) used primers that recognize both angiogenin-3 and angiogenin-4, and RNAs that had been isolated from captured crypt epithelium, villus epithelium, or mesenchymal populations from the villus core. The qRT-PCR analysis indicated that the microbially-regulated ‘angiogenin’ was produced in epithelial cells located at the base of crypts of Lieberkuhn (Hooper, et al., Science, 291, 881 (2001); and Hooper, et al., Nature Immunol, 4, 269 (2003)).

To test the hypothesis that angiogenin-4 expression occurs in Paneth cells, we used LCM to isolate cells located at the base of jejunal crypts from (a) germ-free adult (12 week old) transgenic mice with an attenuated diphtheria toxin-A fragment (tox 176)-mediated Paneth cell lineage ablation (CR2-toxl76 mice) (Garabedian, et al., J. Biol. Chem., 272, 23729 (1997), and (b) their age and gender-matched germ-free normal littermates. qRT-PCR using angiogenin-4-specific primers revealed that angiogenin-4 mRNA levels are 10-fold higher in RNA purified from crypt base epithelial cells of normal mice compared to CR2-tox176 littermates (FIG. 10).

A follow-up study was conducted using conventionally raised NMRI mice. Three cellular pools were harvested by LCM: Paneth cells alone; epithelial cells from the upper crypt and villus (a Paneth cell-minus fraction); and mesenchyme retrieved from the villus core and the peri-cryptal region. The distribution of angiogenin-4 mRNA closely paralleled the distribution of phospholipase A2-the product of the Mom-1 locus and a well-known Paneth cell-specific gene product (data not shown).

Part II—Examples 5-13 correspond to section II of the detailed description and utilize the following materials and methods:

Materials and Methods

Animals. C57BL/6J (B6) WT and Ragl−/−mice were purchased from The Jackson Laboratory. B6 peroxisome proliferator-activator receptor-α (Ppara) -/- mice were kindly provided by F. J. Gonzales (National Institutes of Health, Bethesda). Fasting-induced adipocyte factor (Fiaj)+/− heterozygotes on a mixed B6: 129/Sv background were generated as described below, and Fiaf+/+, Fiaf +/−, and Fiaf−/−littermates, obtained from crosses of Fiaf+/− heterozygotes were compared. Animals were genotyped by using PCR in accordance with methods known in the art.

Conventionally raised (CONV-R) wild-type and knockout mice were rederived as germ-free (GF) as described (L. V. Hooper, et al., Methods in Microbiology, 31, 559 (2002)). GF animals were maintained in gnotobiotic isolators, under a strict 12-h light cycle (lights on at 0600 hours), and fed an autoclaved chow diet (B & K Universal, East Yorkshire, U.K.) ad libitum. All manipulations of mice were performed by using protocols approved by the Washington University Animal Studies Committee.

Colonization of GF Mice—The cecal contents of each 8-week-old CONV-R mouse were resuspended in 10 ml of sterile PBS, and 2-ml aliquots were spread on the fur of 7- to 10-week-old GF recipients. The resulting conventionalized (CONV-D) mice were housed in gnotobiotic isolators for 10-28 d under the same conditions and fed the same diet as their GF counterparts.

CONV-R animals were maintained in microisolator cages in a specified pathogen-free state in a barrier facility on the autoclaved B & K diet. They were transferred to gnotobiotic isolators 2 weeks before they were killed at 8-10 weeks of age to mimic the housing conditions of GF and CONV-D mice.

Eight- to 10-week-old GF mice were orally gavaged with 10⁹ Bacteroides thetaiotaomicron strain VPI-5482. Colonization density in the distal intestine, cecum, and colon ranged from 10⁸ to 10¹¹ colony-forming units/ml luminal contents, as defined by culturing samples of luminal contents on BHI blood agar for 2-3 d at 37° C. under anaerobic conditions.

Measurement of Total Body Fat Content and Metabolic Rate (Oxygen Consumption)—Total body fat content was determined 5 min after mice were anesthesized with an i.p. injection of ketamine (10 mg/kg body weight) and xylazine (10 mg/kg). The protocol used for dual-energy x-ray absorptiometry (Lunar PIXImus Mouse, GE Medical Systems, Waukesha, Wis.) has been described in C. Bernard Mizrachi, et al., Arterioscler. Thromb. Vasc. Biol., 22, 961 (2002).

Oxygen consumption was determined in conscious, individually caged mice, in a fed state, by using open-circuit indirect calorimetry (single-chamber small-animal Oxymar system, Columbus Instruments, Columbus, OH). Animals were allowed to adapt to the metabolic chamber for 20 min before VO₂ was measured every 30 s for 1 h.

SYBR-Green-Based Real-Tie Quantitative RT-PCR (qRT-PCR). RNA was isolated as described in the art and reverse-transcribed by using SuperScript II and dT₁₅ primers lo (Invitrogen). qRT-PCR assays were performed 25-μl reactions that contained cDNA corresponding to 1 ng of total RNA and 900 nM gene-specific primers (Table 1). All assays were performed in triplicate with an ABI Prism 7700 Sequence Detector (Applied Biosystems). Data were normalized to L32 RNA (ΔΔ_{T} analysis).

Analysis of Lipoprotein Lipase (LPL). LPL activity in epididymal fat pads was determined according to P. H. Iverius and A. M. Ostlund-Lindquist Methods Entynol, 129, 691 (1986).

Statistically significant differences were determined by using Student's t tests. Comparisons between more than two groups of mice were made by a one-way ANOVA followed by Tukey's post hoc multiple comparison test.

### EXAMPLE 5

Comparisons of 8-10 week old male C57B1/6J (B6) GF mice raised in the absence of any microorganisms (germ-free; GF; with mice that harbored a microbiota beginning at birth revealed that the latter contain 42% more total body fat, as defined by dual energy X-ray absorptiometry (DEXA; FIG. 14A). Epididymal fat pad weights were also significantly greater (47%; FIG. 14B). The increase in body fat observed in CONV-R animals is intriguing given that their daily consumption of a standard rodent chow diet (57% carbohydrates, 5% fat) was 29% less than their GF counterparts (FIG. 14C).

A 14d colonization of 8-10 week old male GF B6 recipients with an unfractionated microbiota harvested from the distal intestines (cecums) of adult CONV-R donors, a process known as ‘conventionalization’, produced a dramatic 57% increase in their total body fat content (FIG. 14A), and a 61% increase in epididymal fat weight (FIG. 14B). The increase in body fat was associated with a 7% decrease in lean body mass resulting in no significant differences in total body weight between the two groups (23.5±2.6 g (GF) versus 23.4±2.6 g (CONV-D); n=21; p>0.05). Fasting serum triglyceride values were similar (p>0.05) in both GF and CONVentionalizeD (CONV-D) mice (data not shown).

A similar increase in total body fat content was observed after a shorter, 10d conventionalization (66%; p>0.05 compared to 14d). A more prolonged conventionalization (28d) did not produce further increments in total body fat content, or in epididymal fat pad weight (data not shown). The increased fat storage produced by a 14d conventionalization also occurred in the face of decreased chow consumption (27% lower than GF; FIG. 14C).

These effects were not unique to males: CONV-D B6 females exhibited increases in body fat (85%) and reductions in lean body mass (9%) that were not significantly different from age- matched males (p>0.05). In addition, the fat storage phenotype was not limited to the C57B1/6J inbred strain: a 14d conventionalization of 8 week-old male NMRI mice produced a 90% increase in total body fat content (p<0.01) and a 31% decrease in chow consumption (p<0.05).

Sequence-based 16S rDNA enumeration studies of the cecal microbiota revealed great similarities in the fractional representation of the predominant species in CONV-R donors and CONV-D B6 recipients (FIG. 19; Table S1). As in many humans, Bacteroides and Clostridium were the most prevalent genera. We colonized B6 mice for 2 weeks with the sequenced B. thetaiotaomicron strain (VPI-5482), to determine whether a single saccharolytic bacterial species could, by itself, effect host fat storage. A two-week colonization of the adult B6 GF mouse gut produced a statistically significant increase in total body fat content, although the magnitude of the increase was less than that obtained with an unfractionated mouse cecal microbiota (23% versus 57%, respectively; n=10 mice/group; p<0.01).

### EXAMPLE 6

Because the microbiota-mediated increase in body fat content was not due to increased chow consumption, open-circuit indirect calorimetry was performed to determine whether it reflected decreased energy expenditure. This explanation was excluded when we found that the leaner GF mice had a metabolic rate (VO₂) that was 27% lower than age- and gender-matched (male) B6 mice conventionalized for 14d (p<0.01; FIG. 14D). CONV-D mice had VO₂ values that were not significantly different from age- and gender-matched CONV-R animals (FIG. 14D).

The increase in VO₂ observed with conventionalization could reflect increased metabolic rate in the host and/or the metabolic contribution of their recently acquired microbial community. There are no available methods for measuring the metabolic activity of the microbiota in vivo. However, microanalytic biochemical assays of freeze-clamped gastrocnemius muscle and liver revealed significant increases in the steady state levels of TCA cycle intermediates in CONV-D versus GF animals. Despite this evidence of increased cycle activity, there were no significant alterations in tissue high-energy phosphate stores (n=5 animals/group). Increasing oxygen consumption without increasing high-energy phosphate stores implies the presence of futile cycles, a biochemical correlate of inefficient metabolism in the host.

Leptin is an adipocyte-derived hormone whose expression correlates with adipocyte lipid content (M. Maffei et al., Proc Natl Acad Sci USA, 92, 6957 (1995)). Moreover, leptin is known to reduce food intake and increase energy expenditure in mice (M. A. Pelleymounter, et al., Science, 269, 540 (1995)). Fourteen days after colonization, CONV-D animals had 3-fold higher circulating levels of leptin compared to their GF counterparts (FIG. 15A). This increase in leptin was proportional to the increase in body fat (r²=0.977), and provides one potential explanation for the higher oxygen consumption and reduced food intake observed after a two-week colonization.

The increase in fat content was also accompanied by statistically significant elevations in fasting glucose and insulin levels (FIG. 15A), and an insulin-resistant state, as defined by glucose- and insulin-tolerance tests (FIG. 15B, C).

### EXAMPLE 7

Glucose and insulin are known to induce expression of lipogenic enzymes in the liver (H. C. Towle, Proc Natl Acad Sci USA, 98, 13476 (2001)). A 14d conventionalization of GF mice produced a 2.3-fold increase in liver triglyceride content (FIG. 16A, B), but no appreciable changes in total liver free fatty acids or cholesterol (p>0.05; data not shown). qRT-PCR assays confirmed that conventionalization was accompanied by statistically significant elevations in liver mRNAs encoding two key enzymes in the de novo fatty acid biosynthetic pathway, acetyl-CoA carboxylase (AccI) and fatty acid synthase (Fas) (FIG. 16C).

Sterol response element binding protein 1 (SREBP-1) and carbohydrate response element binding protein (ChREBP), two basic helix-loop-helix/leucine zipper transcription factors, mediate hepatocyte lipogenic responses to insulin and glucose, respectively, and appear to act synergistically (R. Dentin et al., J Biol Chem, 279, 20314 (2004)). Both Accl and Fas are known targets of ChREBP and SREBP-1 (H. C. Towle, supra. qRT-PCR assays of liver RNAs revealed that conventionalization increases liver ChREBP mRNA, and to a lesser extent SREBP-1 mRNA levels (FIG. 16C).

ChREBP is translocated from the cytoplasm to the nucleus after it is dephosphorylated by the serine/threonine phosphatase PP2A (H. Yamashita et al., Proc Natl Acad Sci USA, 98, 9116 (2001); T. Kawaguchi et al., Proc Natl Acad Sci USA, 98, 13710 (2001)). PP2A, in turn, is activated by xylulose-5-phosphate (Xu5P) (T. Kabashima et al., Proc Natl Acad Sci USA, 100, 5107 (2003)), an intermediate in the hexose mono-phosphate shunt. Mice colonized with a microbiota had elevated levels of liver Xu5P compared to their GF counterparts (1.6±0.4 versus 2.6±0.3 μmol/g wet weight of liver; p<0.01), and more nuclear-localized ChREBP (FIG. 16D).

The applicants have obtained direct biochemical evidence that the presence of the microbiota promotes increased monosaccharide uptake from the gut. GF mice and their conventionalized counterparts (n=4/group) were given a single gavage of 100 μl of a mixture of 5 mM glucose and 0.2 mM 2-deoxyglucose, sacrificed 15 min later, and 2-deoxyglucose 6-phosphate levels were measured in the distal intestine. Levels were 2-fold higher in CONV-D mice (1.15±0.013 versus 0.55±0.04 pmol/lg protein; p<0.001). Once taken up into the intestine, transfer of monosaccharides to the portal circulation is facilitated through an additional effect of the microbiota: we have shown previously that conventionalization results in a doubling of the density of capillaries that underlie the small intestinal villus epithelium to levels equivalent to that of age-matched CONV-R animals (T. S. Stappenbeck, et al., Proc Natl Acad Sci USA, 99, 15451 (2002)).

Together, these findings are consistent with an increase in processing of dietary polysaccharides by microbial glycosylhydrolases in CONV-D mice, increased delivery of absorbed monosaccharides (and short chain fatty acids) to their livers, and increased trans-activation of lipogenic enzymes by CHREBP and perhaps SREBP-1.

The increased hepatic triglyceride levels could not be ascribed to increased delivery of lactate generated by the microbiota, since serum lactate levels were higher in GF mice (9.22±1.61 mM; n=21) compared to their CONV-D counterparts (5.74±1.66 mM, n=16 p<0.001), and there were no detectable changes in hepatic monocarboxylate transporter-1 mRNA levels (data not shown).

### EXAMPLE 8

The DNA content of epididymal fat pads recovered from GF and CONV-D mice were not significantly different. This finding, together with histochemical studies allowed the applicants to conclude that the microbiota-induced increase in epididymal fat pad weight reflected adipocyte hypertrophy (FIG. 17A). qRT-PCR analyses of fat pad RNA revealed that neither biomarkers of lipogenesis (Acc1, Fas) or adipogenesis (aP2, Ppar-γ) were significantly changed following conventionalization (FIG. 17B).

Lipoprotein lipase (LPL) is a key regulator of fatty acid release from triglyceride-rich lipoproteins in muscle, heart, and fat (K. Preiss-Landl, et al., Curr Opin Lipidol 13, 471 (2002)). Increased adipocyte LPL activity leads to increased cellular uptake of fatty acids and adipocyte triglyceride accumulation. In white fat, LPL is regulated post-transcriptionally by nutritional status: fasting reduces and re-feeding increases enzyme activity (M. Bergo, et al, Biochem J 313, 893 (1996). Intriguingly, we found that a 14d conventionalization increased LPL activity 122% in epididymal fat pads (FIG. 17C). Moreover, the increase was not confined to fat: enzymatic assays of heart revealed a 99% increase with conventionalization (FIG. 17C). Increased insulin levels produce reductions in muscle LPL activity (H. Lithell, Atherosclerosis 30, 89 (1978)). Therefore, our findings indicated that the microbiota induces the observed general increase in LPL through another mechanism.

Fasting-induced adipose factor (Fiaf), also known as angiopoietin-like protein 4, is produced by brown and white fat, liver, as well as intestine (S. Kersten et al., J. Biol Chem 275, 28488 (2000); J. C. Yoon et al., Mol Cell Biol 20, 5343 (2000); L. V. Hooper et al., Science 291, 881 (2001)). This secreted protein is a potent inhibitor of LPL in vitro (IC₅₀=200 nM; (K. Yoshida, et al., J Lipid Res, 43, 1770 (2002)). RT-PCR analysis of intestinal Fiaf expression during postnatal period disclosed that the gene is induced in GF mice during the suckling-weaning transition. Induction does not occur in CONV-R animals, producing significantly lower levels of Fiaf mRNA in adult CONV-R versus GF intestine (FIG. 20). During the suckling-weaning transition, the diet switches from lipid/lactose-rich mother's milk to low fat/polysaccharide-rich chow, with coincident expansion of the microbiota and a shift from facultative to obligate anaerobes (e.g., Bacteroides). These developmental studies suggested that Fiaf could provide a signal that links conventionalization with a change in host fuel partitioning.

qRT-PCR assays disclosed that conventionalization of adult GF mice suppressed Fiaf expression in their small intestines (ileum), but not in their livers or white fat (FIG. 17D). Follow- up qRT-PCR studies of laser capture microdissected intestinal crypt and villus epithelium and the mesenchyme established that microbial suppression of Fiaf occurs in differentiated villus epithelial cells.

These findings suggest that the microbiota acts to stimulate hepatic triglyceride production through effects mediated by transcription factors such as ChREBP, and to promote LPL-directed incorporation of these triglycerides into adipocytes through transcriptional suppression of an intestinal epithelial gene encoding a circulating LPL inhibitor. We tested this hypothesis by generating mice with a null Fiaf allele (FIG. 17E) and re-deriving them as GF.

Eight week-old male GF Fiaf-l-mice have 67% higher epididymal fat pad LPL activity than GF littermates containing the wild-type Fiaf allele (p<0.01), confirming that Fiaf is an important inhibitor of this lipase in vivo. Conventionalization of GF knockout mice did not produce significant changes in LPL activity in fat pads (or heart) (p>0.05; n=10 animals).

GF Fiaf-l- animals have the same amount of total body fat as their age- and gender-matched CONV-D (Fiaf-suppressed) wild-type littermates (12.8±1.1 versus 14.2±1.9, p>0.05). Moreover, a 14d conventionalization of already Fiaf-deficient GF knockout animals produced only minor increases in total body fat (10±8% versus 55±16% in wild-type littermates; FIG. 17F). Fiaf+/−heterozygotes had an intermediate increase (33±12%). These results establish the importance of Fiaf as a prominent mediator of microbial regulation of peripheral fat storage.

### EXAMPLE 9

The mechanisms by which the mammalian gut microbial community influences host biology and gene expression, such as the suppression of Fiaf remain almost entirely unknown. The zebrafish, Danio rerio, has several unique features that make it an attractive model organism for analyzing these pathways. First, zebrafish larvae and their digestive tracts are transparent from the time of fertilization through early adulthood, allowing in vivo observation of the developing gut (M. S. Pack, et al., Development, 123, 321 (1996); S. A. Farber, et al., Science, 292, 1385 (2001)) and its resident microorganisms (J. M. Davis, et al., Immunity, 17, 693 (2002); A. M. van der Sar, et al., Cell Microbiol.5, 601 (2003). Second, zebrafish development occurs rapidly. Larvae hatch from their chorions at ˜3 days post-fertilization (dpf). By 5 dpf, the yolk is largely absorbed and gut morphogenesis has proceeded to a stage that supports feeding and digestion (M. Pack, et al., supra; S. A. Farber, et al., supra). Third, the organization of the zebrafish gut is similar to that of mammals. As in mice and humans, the intestinal epithelium undergoes renewal throughout life. A proliferative compartment, analogous to the mammalian crypt of Lieberkuhn, is located at the bases of intestinal villi (K. N. Wallace, et al., Mech Dev, 122, 157 (2005)). Epithelial progenitors give rise to cell types encountered in other vertebrates, including absorptive enterocytes, mucus-producing goblet cells, and an enteroendocrine lineage (M. L. Pack, et al., supra). Fourth, GF larvae of other fish species have been produced by aseptically removing gametes from adults, and treating fertilized eggs with germicidal agents while they develop in the axenic environment provided by their protective chorions (J. A. Baker, et al., Proc. Soc. Exp. Biol. Med., 51, 116 (1942); T. J. Trust, Appl. Microbiol., 28, 340 (1974); R. Lesel, R., et al., Ann Hydrobiol, 7, 21 (1976)). Finally, the capacity to perform forward genetic analyses in a vertebrate that is transparent in the postembryonic period has already led to the identification of mutants with defects in gut development (M. L. Pack, et al., supra; A. N. Mayer, et al., Development, 130, 3917 (2003)) and digestive physiology (S. A. Farber, et al., supra). Reverse genetic analysis using antisense morpholino oligonucleotides (A. Nasevicius, et al., Nat. Genet., 26, 216 (2000) or target-selected mutagenesis (E. Wienholds, et al., Genome Res., 13, 2700 (2003)), as well as chemical screens (R. T. Peterson, et al., Proc. Natl. Acad Sci. USA, 97, 12965 (2000); S. M. Khersonsky, et al., J. Am. Chem. Soc., 125, 11804 (2003)), provide additional means for identifying molecular mediators of host-microbial interactions. The imminent completion of the zebrafish genome will facilitate many of these approaches (http://www.sanger.ac.uk/Projects/D-rerio/).

To investigate the impact of indigenous microbial communities on zebrafish biology, the applicants developed procedures for producing and rearing GF zebrafish and for conventionalizing them or colonizing them with single components of the normal zebrafish or mammalian gut microbiota.

CONV-R zebrafish (C32 inbred strain) were reared through 14 dpf at a density of ˜0.4 individuals per milliliter static water that had been harvested from tanks in a recirculating zebrafish aquaculture facility. Animals were subsequently maintained at ˜0.03 individuals/mL static water through 28 dpf, and then moved to recirculating tanks. CON-R zebrafish were fed rotifers (Aquatic Biosystems) beginning at 3 dpf, followed by brine shrimp (Aquafauna Bio-Marine) beginning at 14 dpf, and then advanced to a diet of brine shrimp, TetraMin flakes (Tetra), and Hikari micropellets (Hikari) at 28 dpf.

To generate and rear GF zebrafish, adult male and female CONV-R zebrafish (C32 inbred strain) were collected, euthanized in 3-amino benzoic acid ethyl ester (Sigma; final concentration 1 mg/mL; 10 min exposure), and then immersed in a bath of 10% polyvinylpyrrolidone (PSS Select) for 2 min at room temperature. After carefully opening the abdominal walls of the males to avoid rupturing their intestines, testes were removed, placed in a sterile 1.5 mL Eppendorf tube containing 500 μl of sterile Hanks solution (4° C.), and dissociated with a sterile pestle. The abdominal walls of gravid females were opened in a similar fashion, ovaries were ruptured, and eggs removed from the body cavity with a sterile Pasteur pipette. Eggs were fertilized in vitro with the collected sperm in sterile plastic 60 mm diameter Petri dishes (10 min incubation at room temperature). Fertilized eggs were subsequently washed three times in sterile water (3 min/cycle at room temperature), and incubated for 6 h at room temperature in ˜10 mL of a sterile solution of 0.3 mg/mL marine salt (Coralife), 100 μg/mL ampicillin, 5 μg/mL kanamycin, and 250 ng/mL amphotericin B. Embryos were then washed at room temperature in 0.1% polyvinylpyrrolidone (PSS Select) for 2 min, rinsed 3 times with sterile water at room temperature, immersed in 0.003% sodium hypochlorite (Novel Wash Co.) for 20 min at room temperature, and simultaneously transferred into plastic gnotobiotic isolators (Standard Safety Equipment). Once inside the gnotobiotic isolators, zebrafish embryos were rinsed 3 times with sterile water, and then reared in these isolators in a static solution of gnotobiotic zebrafish medium [GZM; 0.3 g/L marine salt (Coralife); neutral pH buffer (Bullseye 7.0, Wardley)] at a density of 0.4 individuals/mL GZM, in 400 mL glass beakers. Each day, 50% of the GZM in each beaker was replaced with fresh media. Water temperature was maintained at 28° C. using an external K-MOD 107 heating system (Allegiance Healthcare). Beginning on 3 dpf, the solution was supplemented with dissolved autoclaved chow (ZM000, ZM Ltd; 20 mg dry weight/L). To insure that the isolators were free of contaminating bacteria or fungi, their inside surfaces were routinely swabbed, and aliquots of GZM containing dissolved food were removed from beakers, and cultured aerobically and anaerobically at 28° C. and 37° C. in three different media (nutrient broth, brain/heart infusion broth, and Sabouraud dextrose broth).

To generate conventionalized animals, water was collected from recirculating tanks in a conventional zebrafish aquaculture facility, and passed through a 5 μm pore diameter filter (Millipore). Microbial density in the filtrate was defined by culture under aerobic and anaerobic conditions at 28° C. on brain/heart infusion blood agar. 10⁴ CFU of bacteria were added per mL GZM containing 3 dpf GF zebrafish.

In some experiments, GF animals were colonized at 3 dpf with a single bacterial species. Aeromonas hydrophila (ATCC 35654) and Pseudomonas aeruginosa (strain PA01) were grown overnight under aerobic conditions in tryptic soy broth (TSB) at 30° C., and in nutrient broth at 37° C., respectively, and then added to beakers containing 3 dpf GF zebrafish at final concentrations of 10⁴ CFU/mL GZM.

GF and CONV-R zebrafish started to feed at 5 dpf and were indistinguishable macroscopically through −8 dpf (FIG. 24 A, B). At 9 dpf, GF animals began to develop a stereotyped, rapidly progressive epidermal degeneration phenotype manifested by epidermal opacity, loss of epidermal integrity, and sloughing of epidermal cells (FIG. 24 D, E). Mortality was 100% by 20 dpf (n=824 zebrafish scored). The phenotype was rescued by exposing 3 dpf or 6 dpf GF animals to the microbiota contained in water obtained from a conventional zebrafish aquaculture facility (FIG. 24F, plus data not shown). This finding indicates that the degenerative changes observed in late larval stage GF animals are not due to irreversible insults acquired earlier in development. Our observations that (i) animals conventionalized at 3 dpf and fed the same autoclaved diet can live to adulthood (≧42 dpf), and (ii) unfed GF animals do not develop this phenotype through 12 dpf (n=44 scored) suggest that this phenomenon is due to deleterious effects of exposure to autoclaved chow that are ameliorated by the presence of the microbiota. If activated carbon filters are included in the static rearing vessels, GF zebrafish do not develop this epidermal degeneration phenotype, and can be reared into adult stages.

GF zebrafish harvested at 6 dpf, and animals conventionalized at 3 dpf and sacrificed 3 days later (CONV-D), have a similar gross morphology (FIG. 24B, C). Additionally, GF zebrafish at 6 dpf exhibit no statistically significant differences in their average body length compared to age-matched CONV-D and CONV-R larvae [4.06±0.11 mm (GF); 4.09±0.11 mm (CONV-D); and 4.02±0.15 mm (CONV-R); P>0.3 for each comparison based on Students t-test]. Given the phenotype observed in GF fish ≧9 dpf, the analysis was focused of the effects of the microbiota on host biology using 6 dpf animals.

The zebrafish is a stomachless teleost: its pharynx is continuous with the proximal intestine (segment 1), which is largely responsible for lipid absorption. Segment 2 of the intestine (FIG. 24A) is involved in absorption of other macromolecules, while a short distal domain (segment 3) is postulated to participate in water and ion transport (H.W. Stroband, et al., Cell Tissue Res, 187, 181 (1978); J. Noaillac-Depeyre, et al., Tissue Cell, 8, 511 (1976); H. W. Stroband, et al., Histochemistry, 64, 235 (1979)).

The proximal intestine, liver, pancreas, and gallbladder of GF and CONV-D animals were indistinguishable, whether judged by examination of wholemount preparations (FIG. 24B, C), serial hematoxylin and eosin stained sections (e.g., FIG. 24 G, J; n=20-34 animals/treatment), or by transmission EM (data not shown).

GF mice have reduced rates of epithelial proliferation in their intestinal crypts of Lieberkuhn compared to their CONV-R or CONV-D counterparts. A similar situation occurs in zebrafish. Quantitative BrdU labeling studies disclosed that the fractional representation of S-phase cells in the intestinal epithelium was significantly greater in 6 dpf CONV-D and CONV-R zebrafish compared to GF animals (P<0.0001 in each case based on Student's t-test; n≧12 animals/condition; FIG. 25A-C). No significant differences were observed in the underlying mesenchyme/muscle (FIG. 25C). The increase in epithelial proliferation was not accompanied by a statistically significant change in apoptosis, as judged by TUNEL assays of epithelium and underlying mesenchyme/muscle in the same animals (data not shown; P>0.3 for all comparisons).

To gain additional insights about the mechanisms underlying these microbiota-associated phenotypes, as well as other aspects of host physiology affected by gut microbes, the applicants conducted a broad, functional genomics-based analysis of gene expression in the digestive tracts of 6 dpf GF, CONV-D, and CONV-R zebrafish. Comparisons were performed using DNA microarrays containing 16,228 65-mer oligonucleotides representing zebrafish genes and ESTs (Sigma-Genosys Zebrafish Oligonucleotide Library). RNA was isolated from the pooled digestive tracts of 30 animals per treatment group. Two independently generated cohorts of animals were evaluated for each condition (i.e., a total of 60 animals). These “biological duplicates”, together with Cy3- and Cy5-labeled probe dye swap controls, produced a total of four DNA microarray datasets for each of the two comparisons performed (i.e., CONV-D versus GF; CONV-R versus GF).

Each experiment consisted of pairwise competitive hybridizations from two treatment groups (CONV-D versus GF at 6 dpf, CONV-R versus GF at 6 dpf, 6 dpf versus 10 dpf CONV- R, or 10 dpf versus 20 dpf CONV-R), plus reciprocal dye-swap replicates. Since biological duplicates were generated for each treatment group, a total of four DNA microarrays were utilized per comparison of two treatment groups. Oligonucleotide elements that (i) received “present” calls in all four microarrays and (ii) displayed >1.55 mean signal-to-noise ratio across both dye channels in all four microarray replicates, were identified and all others were excluded. The log₂ ratio of median dye intensities for each remaining element was averaged across all four microarrays. To account for measurement variance among replicate microarrays within an experiment, standard deviations of the averaged log₂ ratios of all remaining elements were averaged to identify the standard deviation for the experiment (SDE) (I. V. Yang, et al. (2002) Genome Biol. 3, research0062).

When considering the results of an experiment, the applicants defined differentially expressed genes as those displaying an average log₂ ratio with an absolute value of greater than 2 SDE, providing ˜95% confidence (GF versus CONV-D 1 SDE=0.501; GF versus CONV-R 1 SDE=0.566). Differentially expressed genes identified in this manner are referred to by the names of their putative mouse or human homologs. Homologies were assigned using the following methods: (i) previous zebrafish gene name assignment, (ii) EST assembly homology (http://zfish.wustl.edu), (iii) Unigene homology (http://www.ncbi.nlm.nih.gov), or (iv) Ensembl gene prediction homology based on corresponding genomic sequence (http://www.sanger.ac.uk/Proiects/D rerio). Functional classification of genes was based in part on the Gene Ontology Consortium database (http://www.geneontology.org). For microarray image files, ScanArray output files, and other MIAME information, see http://gordonlab.wustl.edu/.

Using the criteria described above, the applicants identified 212 genes that exhibited differential expression in both GF versus CONV-D and GF versus CONV-R comparisons. In addition, the applicants referenced zebrafish genes culled from comparisons of GF versus CONV-D and/or GF versus CONV-R animals to our previous DNA microarray datasets of genes differentially expressed in the GI tracts (small intestine, colon, or liver) of adult GF mice versus ex-GF mice colonized with components of the normal mouse intestinal microbiota. Sixty-six homologous genes were identified as responsive to the microbiota in both fish and mice. Expression of 54 of these changed in the same direction (up or down) in both species. Moreover, 59 of the 66 genes were identified in the applicant's analysis of the response of the mouse intestine, and did not occur in mouse liver datasets.

For example, the increased epithelial proliferation associated with the microbiota was manifested by the increased expression of 15 genes involved in DNA replication and cell division. They include thymidylate kinase (Dtymk), four minichromosome maintenance genes (Mcm2, Mcm3, Mcm5, Mcm6), plus origin recognition complex subunit 4 (Orc4l), proliferating cell nuclear antigen (Pcna), and ribonucleotide reductase subunit M2 (Rrm2). Importantly, the zebrafish ortholog of Fiaf was suppressed by the microbiota.

While these studies reveal a wide range of conserved responses of the zebrafish digestive tract to the presence of a microbiota, the nature of this microbiota, and its degree of similarity to microbial communities that reside in the mouse or human gut, had not been previously defined. Therefore, the applicants generated and sequenced libraries of bacterial 16S rDNA amplicons produced by PCR of DNA prepared from the microdissected digestive tracts of CONV-R 6 dpf, 10 dpf, 20 dpf, 30 dpf and adult animals. Since a number of variables can affect the composition of a microbiota (e.g., nutrient supply, aquaculture conditions, as well as developmental stage), we used our sequence data only to identify genus/species that can occur within the zebrafish digestive tract.

The only genera found at all timepoints surveyed were Aeromonas and Pseudomonas. Vibrio and Lactococcus ssp. were also commonly encountered. Comparisons of the digestive tract microbiotas of 6 dpf CONV-D versus CONV-R zebrafish indicated an enrichment of Aeromonas in the former (61% of all sequenced clones in CONV-D versus 0.3% in CONV-R), and of Vibrio in the latter (57% in CONV-R versus 12% in CONV-D).

Previous culture-based enumerations of the intestinal microbiotas of freshwater and marine fish identified Pseudomonas, Aeromonas, Vibrio, and Flavobacterium genera as the most common components, with good, albeit lower, representation of Lactobacillus, Staphylococcus, Acinetobacter, Streptococcus, and Leuconostoc spp. (B. Spanggaard, et al., Environ. Microbiol,. 3, 755 (2001); M. M. Cahill, Microb. Ecol., 19, 21 (1990); E. Ringo, et al., Aquaculture Res., 26, 773 (1995)). Our results also revealed some similarities to the mammalian gut microbiota. For example, the zebrafish microbiota contained members of Bacteroidetes (e.g., Flavobacterium and Flexibacter), a major phylum in mice, humans and other mammals (D. C. Savage, Annu. Rev. Microbiol., 31, 107 (1997), components of Ralstonia and Plesiomonas genera (N. H. Salzman, et al. Microbiology, 148, 3651 (2002); T. Arai, et al., J Hyg. (London) 84, 203 (1980)), as well as a number of lactic acid bacteria (Lactococcus lactis, Lactobacillus fermentum, Leuconostoc citreum, and Weissella confusa).

To determine whether some of the observed evolutionarily conserved host responses to the microbiota exhibited microbial species specificity, the applicants colonized 3 dpf GF zebrafish with individual components of the digestive tract microbiota for 3 days. Two culturable and genetically manipulatable Gram-negative bacterial species were chosen for these monoassociation experiments as representative of the Aeromonas and Pseudomonas genera that were consistently represented in the digestive tracts of 6dpf to adult zebrafish (i.e., A. hydrophila and P. aeruginosa).

RNA was isolated from the pooled digestive tracts of 10 animals per condition at 6 dpf (n=2 groups/condition), and host transcriptional responses were quantified using qRT-PCR. Two control RNAs were used as reference standards: 6 dpf GF and 6 dpf CONV-D digestive tracts (n=30/group; two independent groups/condition to generate biological duplicates). Importantly, the average number of viable organisms recovered from the digestive tracts of CONV-D or monoassociated animals was not significantly different (4.4−8.3×10⁴ CFU/digestive tract; P≧0.26).

The qRT-PCR results showed that the response of some genes—Saal, Mpo, Apob, and Arg2—was robust whether there was colonization with an unfractionated microbiota or with either of the two individual species (FIG. 26A plus data not shown). In contrast, C3 responded to the presence of a normal microbiota and to A. hydrophila, but not to P. aeruginosa (FIG. 26B). Conversely, Fiaf responded to a normal microbiota and P. aeruginosa, but not to A. hydrophila (FIG. 26C). These findings indicate that, as in mice (L. V. Hooper, et al., Science, 291, 881 (2001), at least a subset of zebrafish genes are sensitive to factors represented in only a subset of bacterial components of the gut microbiota.

To facilitate translation of findings in the zebrafish to mammalian models, the applicants have determined whether members of the human/mouse gut microbiota could colonize the zebrafish intestine and elicit evolutionarily conserved host responses. They found that Escherichia coli can colonize the 3 dpf GF zebrafish gut at densities comparable to endogenous community members such as A. hydrophila or P. aeruginosa (i.e., 10⁴/gut at 6 dpf). Furthermore, E. coli is capable of eliciting many of the principal host responses to the gut microbiota in zebrafish (i.e., intestinal epithelial cell proliferation, innate immune response, and promotion of nutrient metabolism). For example, colonization of GF zebrafish at 3 dpf with E. coli results in downregulation of Fiaf by 6 dpf (FIG. 23B).

As noted above, 3 dpf GF zebrafish were placed in a trans-well cell culture dish containing gnotobiotic zebrafish medium (GZM) and autoclaved chow (ZM000, ZM Ltd; 20 mg dry weight per mL). Live E. coli MG1655 in GZM with a similar concentration of fish chow (initial concentration 10⁴ CFU/mL) were placed in the transwell chamber separated from the zebrafish by a filter with 0.4 μm diameter pores. qRT-PCR studies of digestive tract RNA indicated that by 6 dpf, these GF zebrafish displayed Fiaf mRNA levels similar to standard E. coli mono-associated animals raised in the same media conditions (FIG. 23D). The same result was obtained when 3 dpf GF zebrafish were immersed with heat-killed E. coli for 3 days (FIG. 23D).

These methods can be used to identify factors that mediate microbial regulation of Fiaf and host nutrient metabolism by generating transgenic zebrafish that express cyan fluorescent protein (CFP) from zebrafish Fiaf regulatory sequences. These fish can then be exposed to conditioned media, or derived fractions, or microbial extracts, or derived fractions, and the effect on host Fiaf gene expression monitored by monitoring changes in the fluorescent protein reporter using fluorescence imaging methods.

### EXAMPLE 10

Two methods were applied to identify conserved regulatory elements in the 10 kb of DNA sequence 5′ to the transcriptional start site of human, mouse, rat, zebrafish and fugu Fiaf orthologs. First, we searched for novel motifs using PhyloCon (T. Wang, et al., Bioinformatics 19, 2369 (2003)). Two statistically significant motifs were identified: one overlaps with the peroxisome proliferator-activator receptor (Ppar) binding site; the other is similar to the Heb binding site, which contains an E-box (panel A in FIG. 21). Second, we searched the TRANSFAC database (V. Matys et al., Nucleic Acids Res, 31, 374 (2003)) of 466 vertebrate specific transcription factor scoring matrices with PATSER (G. Hertz and G. Stormo, unpublished, http://ural.wustl.edu) for high-scoring binding sites that appear in all five Fiaf orthologs, and in conserved sequence blocks between the human and mouse genes. Over 40 matrices satisfied these two selection criteria (Table 2S), including sites recognized by several fork head domain-containing factors (e.g., HNF3, HNF4α, FKH8), as well as interferon-stimulated response element (ISRE) (FIG. 21).

### EXAMPLE 11

Fiaf was identified during a screen for Ppar-α targets in liver (J. F. Rawls, et al., Proc Natl Acad Sci USA, 101, 4596 (2004)). Ppar-α is an important regulator of energy metabolism in a variety of tissues including intestine, liver, heart and kidney (O. Braissant, et al., Endocrinology 137, 354 (1996)). We found that Ppar-α mRNA levels decrease modestly (1.7±0.2 fold) in the small intestines of CONV-D compared to GF animals, but remain unchanged in their livers and fat pads (p<0.05; see FIG. 22). To directly test the role of Ppar-α in regulating the microbiota-directed change in body fat content and suppression of Fiaf, B6 Ppara knockout mice were re-derived as GF. 8-10 week old male GF Ppara−/−mice had the same amount of total body fat as their age- and gender-matched GF wild-type littermates (FIG. 22). Moreover, Ppara−/−animals had no impairment in their microbiota-induced increase in body fat content (FIG. 22). Finally, qRT-PCR assays of intestinal RNAs isolated from GF and CONV-D wild-type and Ppara−/−mice indicated that the absence of Ppar-α did not prevent transcriptional suppression of Fiaf upon conventionalization (FIG. 22). We concluded that the host fat storage response to the microbiota does not require Ppar-α. A comparable analysis of the role of Ppar-γ could not be performed because Pparg−/−mice die at embryonic day 10.

### EXAMPLE 12

Finding a conserved ISRE element in the orthologous Fiaf genes was intriguing in light of our previous GeneChip analyses of intestinal RNAs which revealed that conventionalization of B6 GF mice regulates expression of a number of genes involved in B- and T-cell responses (J. F. Rawls, et al., Proc. Natl. Acad. Sci. USA, 101, 4596 (2004)). Therefore, we re-derived B6 Rag1−/−deficient mice as GF to determine whether the presence or absence of mature T- and B-cells had an effect on the capacity of the microbiota to increase body fat content or modulate Fiaf. Rag1+/+ and Rag1−/−littermates had equivalent increases in body fat content after a 14d conventionalization (59±16% versus 67±16%; p>0.05) and similar degrees of Fiaf suppression (2.8±0.3 and 3.8±0.3-fold, respectively). Thus, it appears that these cellular components of the adaptive immune system are not required to process signals or metabolic products emanating from the gut microbiota that promote fat storage. Data from Examples 5-12, is depicted in Tables S1, S2 and S3.

TABLE S1
Bacterial genera and species identified in the cecums of a conventionally raised
(CONV-R) donor mouse and four conventionalized (CONV-D) C57B1/6J recipients.

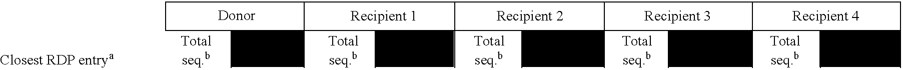

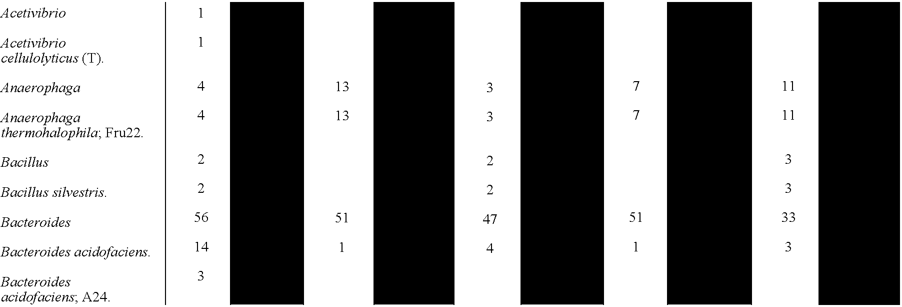

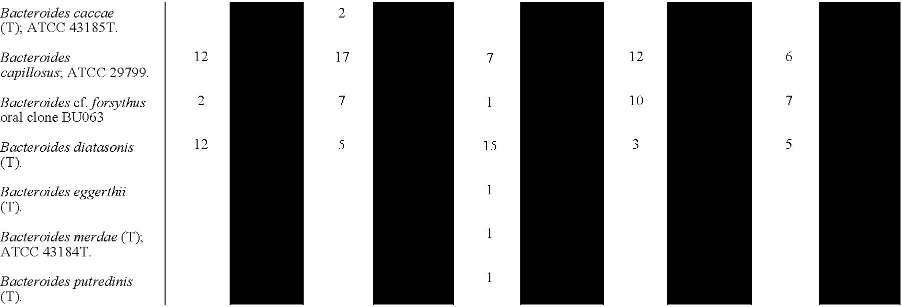

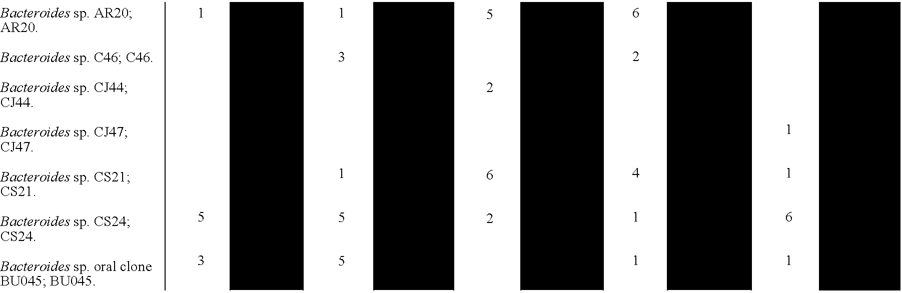

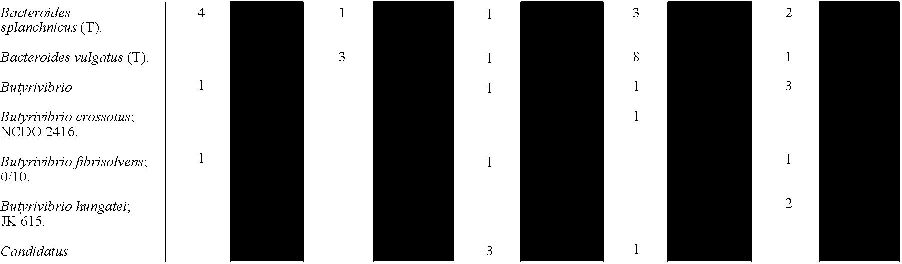

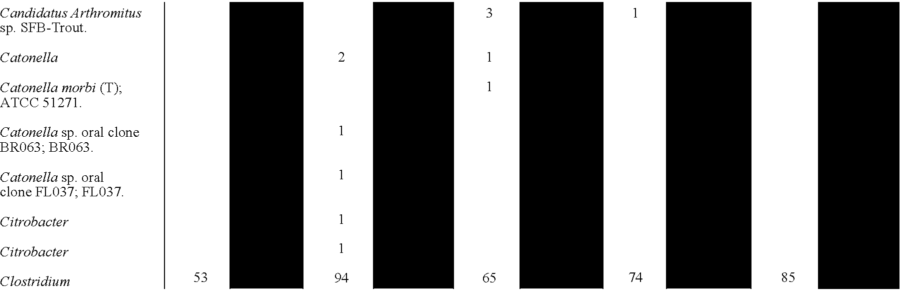

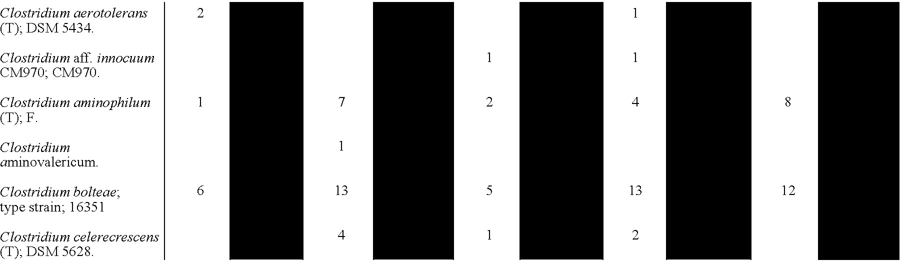

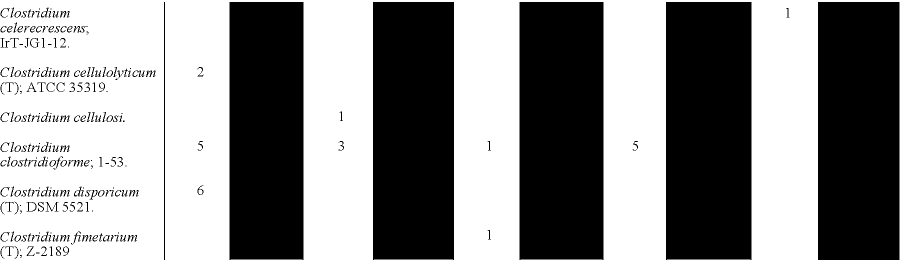

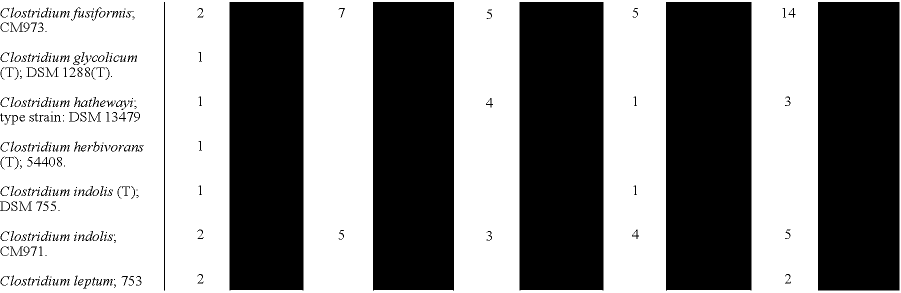

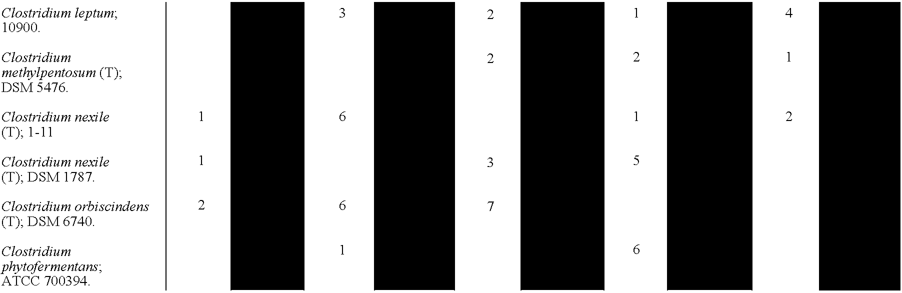

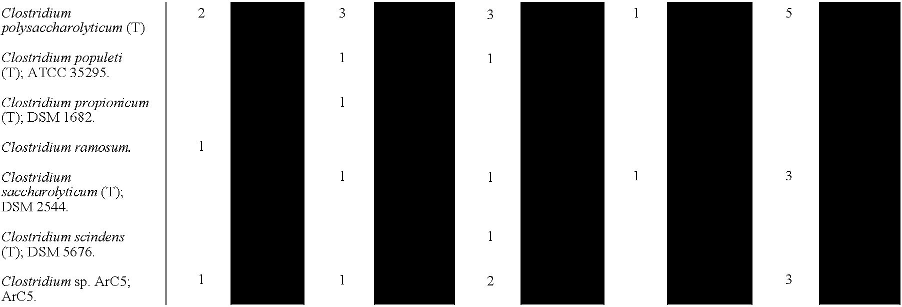

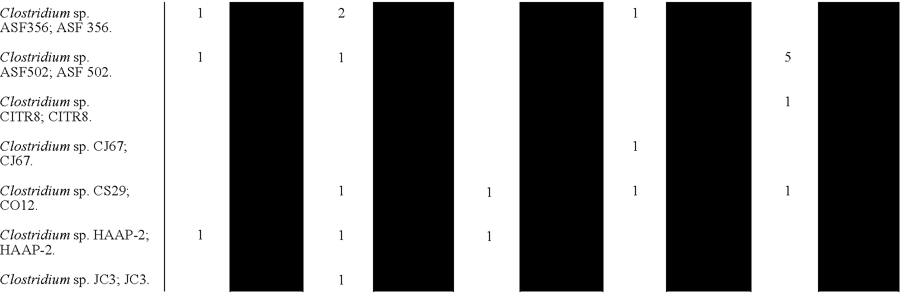

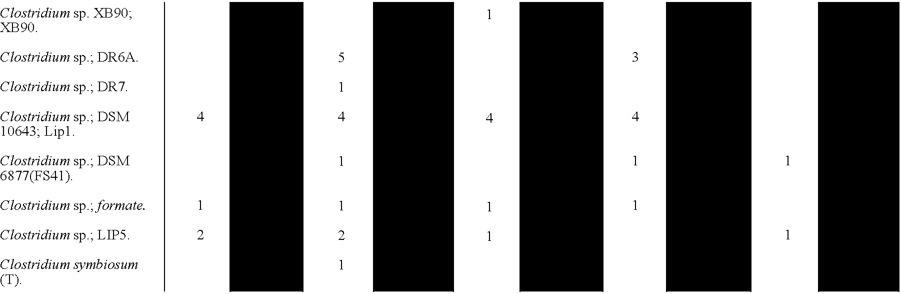

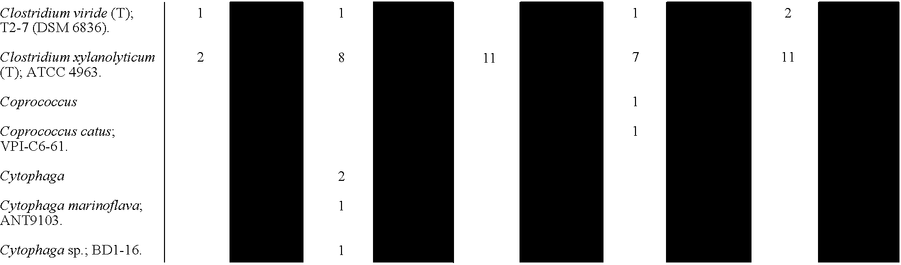

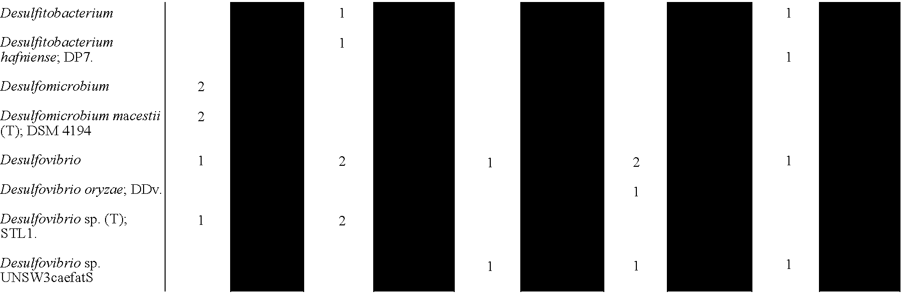

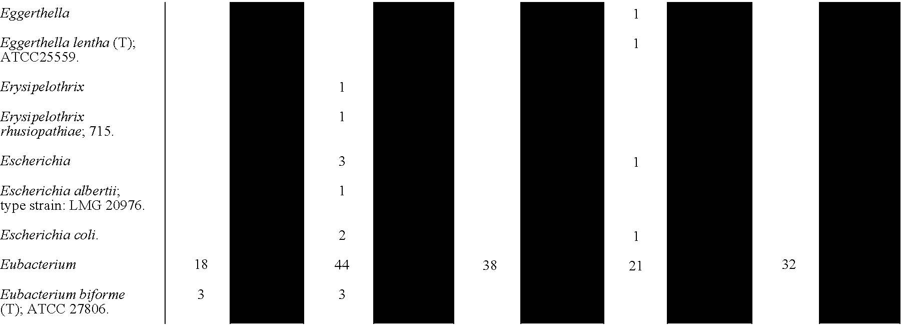

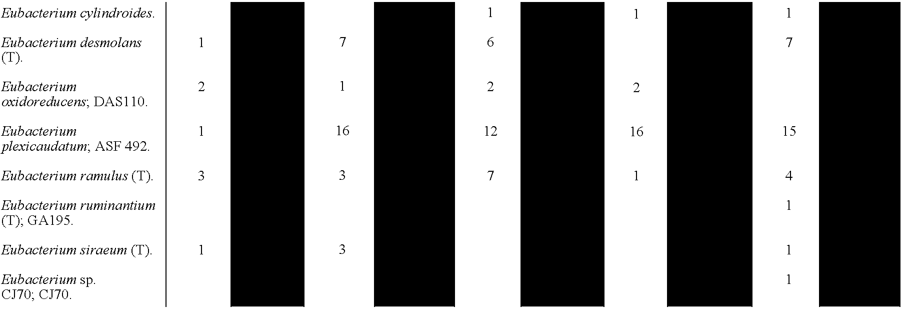

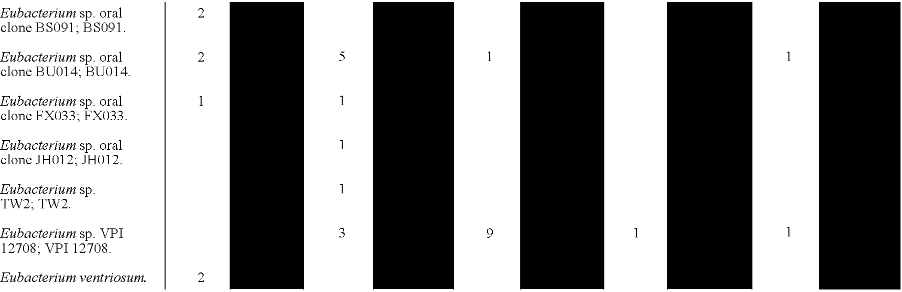

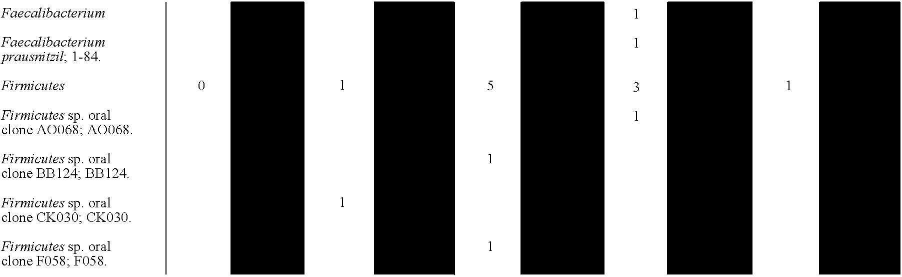

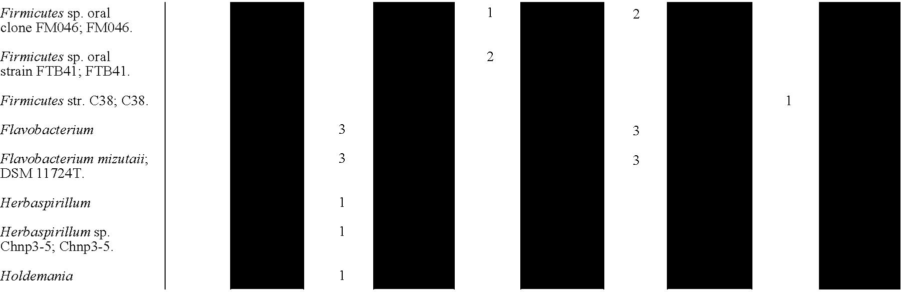

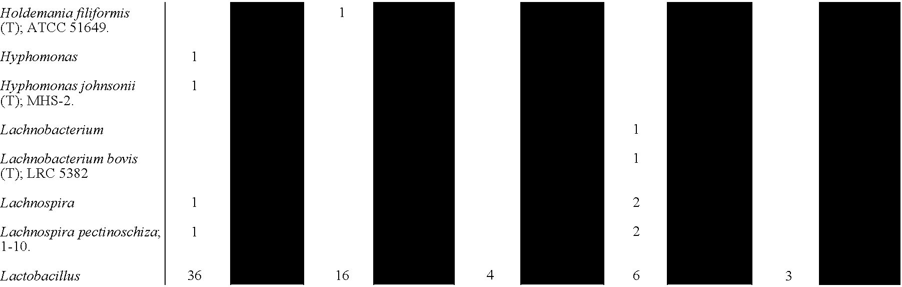

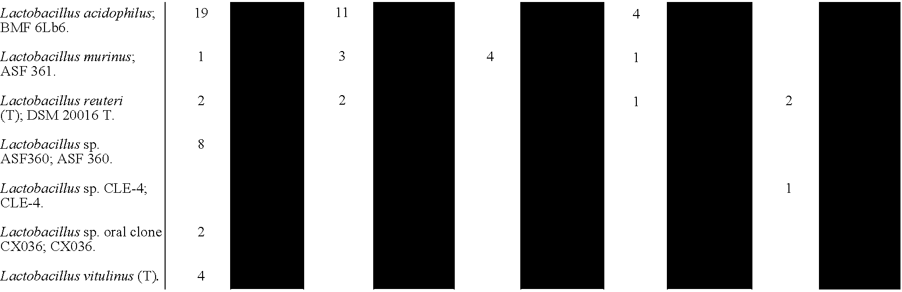

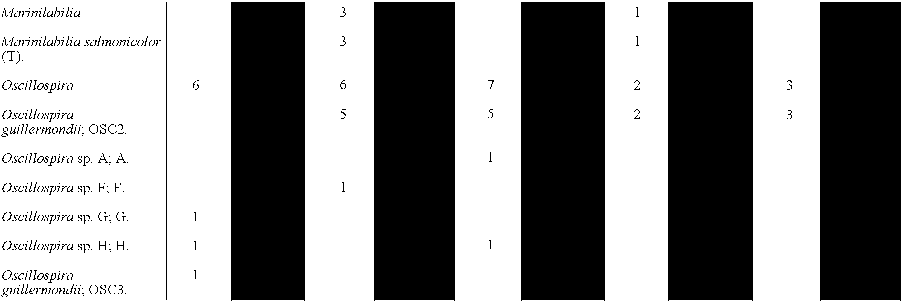

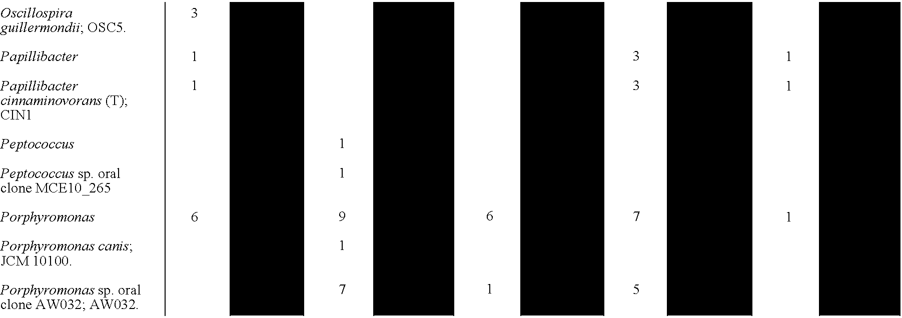

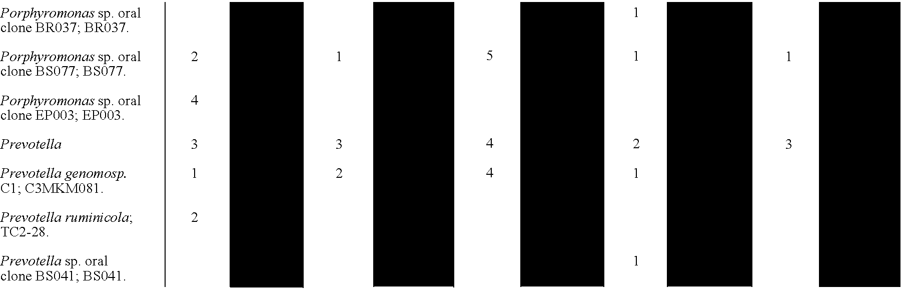

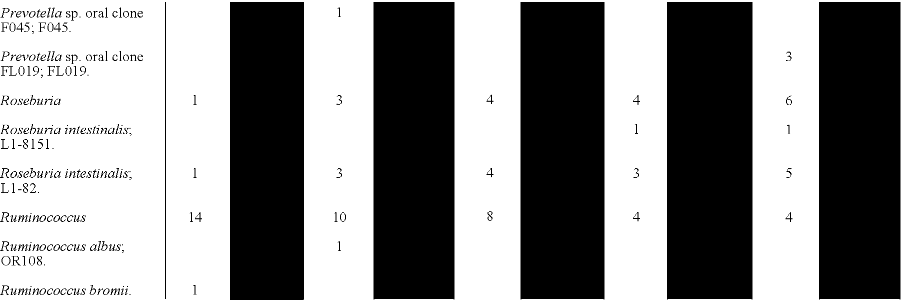

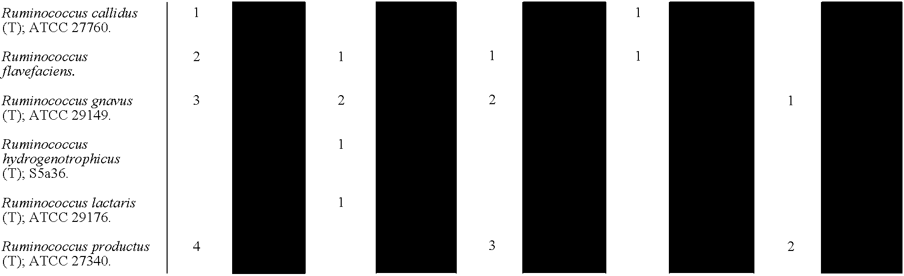

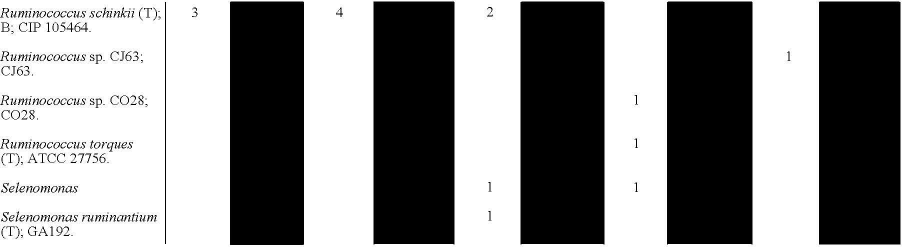

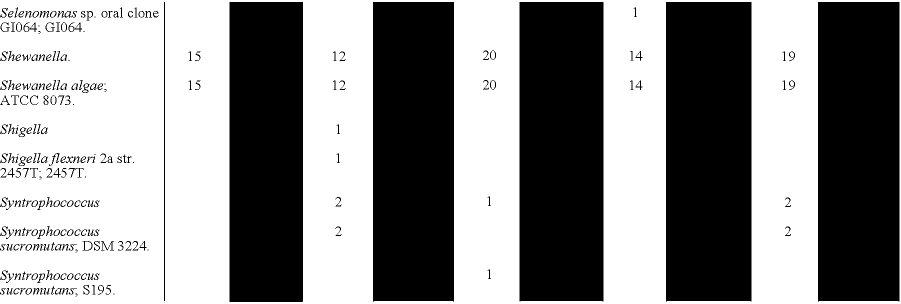

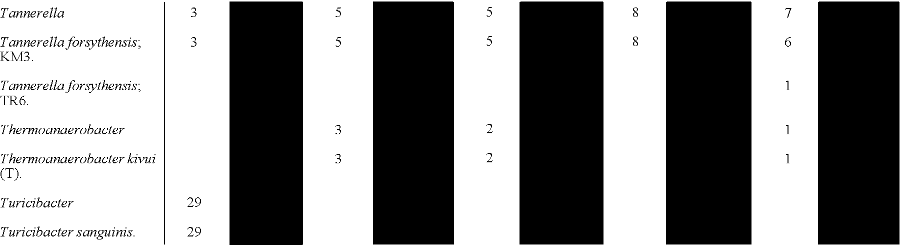

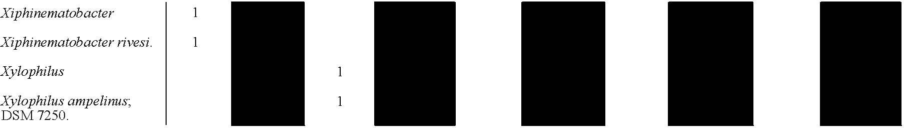

^{a}Bacterial 16S rDNA Ribosomal Database Project (RDP) entries are organized by genus (bold type) with specific RDP entries listed below each genus heading (plain type).
^{b}Total number of 16S rDNA clones that (i) passed the selection criteria described in Materials and Methods, and (ii) were homologous to the respective RDP entry with species or genus information.
^{c}16S clones that are defined as “unidentified” (shaded columns) because their closest relative in RDP is either (i) an entry without species assignment, or (ii) an entry with species or genus assignment but with less than 98% identity to the respective rDNA sequence. These clones are listed in the table according to their closest relative in RDP with species or genus assignment.
# GenBank Accession numbers for the sequences are AY667702-AY668946. Further details of homology analyses are available at http://gordonlab.wustl.edu/.

TABLE S2
Conserved transcription factor binding sites identified in orthologous Fiaf genes.

| | | Number of | | | | | | |
|---|---|---|---|---|---|---|---|---|
| | | Potential | | | | | | |
| TRANSFAC | | Sites | | | | | | |
| Matrices | consensus | H | M | R | Z | F | CHN | Notes |
| AP1_01 | NNNTGAGTCAKCN | 2 | 4 | 1 | 3 | 3 | 1 | Ap1 site, activator protein 1 |
| AP1_C | NTGASTCAG | 5 | 2 | 1 | 6 | 2 | 1 | Ap1 site, activator protein 1 |
| AP4_Q6 | CWCAGCTGGN | 4 | 2 | 2 | 2 | 4 | 1 | Ap4 site, activator protein 4 |
| CEBPGAMMA_Q6 | CTBATTTCARAAW | 1 | 1 | 5 | 9 | 4 | 1 | CCAAT enhancer binding protein |
| CREL_01 | SGGRNTTTCC | 2 | 3 | 4 | 2 | 2 | 1 | C-Rel, overlap with NFkB |
| DR1_Q3 | RGGNCAAAGGTCA | 2 | 2 | 4 | 2 | 2 | 1 | PPAR, HNF4, direct repeat |
| E12_Q6 | RRCAGGTGNCV | 3 | 3 | 2 | 4 | 6 | 1 | E-box |
| E2A_Q2 | NCACCTGYYNCNKN | 2 | 3 | 3 | 5 | 5 | 1 | E-box |
| ETS_Q4 | ANNCACTTCCTG | 3 | 3 | 3 | 4 | 4 | 1 | C-Ets, T-cell, mesodermal cell development |
| FAC1_01 | NNNCAMAACACRNA | 2 | 1 | 5 | 9 | 2 | 1 | Fac1 site, fetal Alz-50 clone 1 |
| FOXD3_01 | NAWTGTTTRTTT | 2 | 7 | 4 | 31 | 5 | 3 | Fork head box D3 |
| FOXO1_01 | NNNWAAAYAAAYANNNNN | 3 | 5 | 4 | 22 | 14 | 2 | Fork head box O1 |
| FOXO4_01 | RWAAACAANNN | 2 | 4 | 4 | 18 | 9 | 1 | Fork head box O4 |
| FOX_Q2 | KAWTGTTTRTTW | 1 | 3 | 5 | 16 | 7 | 1 | Fork head factor |
| GC_01 | NRGGGGCGGGGCNK | 8 | 4 | 4 | 2 | 3 | 2 | GC box |
| GR_Q6 | NNNNNNCNNTNTGTNCTNN | 3 | 1 | 1 | 2 | 1 | 1 | glucocorticoid receptor site |
| HFH8_01 | NNNTGTTTATNTR | 1 | 5 | 5 | 15 | 8 | 1 | HNF3, Fkh8 site |
| HNF3ALPHA_Q6 | TRTTTGYTYWN | 1 | 5 | 4 | 22 | 4 | 1 | HNF3-alpha site |
| HNF3_Q6 | NWRARYAAAYANN | 1 | 6 | 3 | 28 | 7 | 1 | HNF3 site |
| HNF4ALPHA_Q6 | VTGAACTTTGMMB | 2 | 2 | 1 | 5 | 3 | 1 | HNF4-alpha site |
| HSF_Q6 | TTCCMGARGYTTC | 1 | 3 | 3 | 1 | 1 | 3 | Heat shock factor site |
| ICSBP_Q6 | RAARTGAAACTG | 1 | 3 | 4 | 9 | 3 | 1 | ICSBP, Interferon factor binding site |
| IRF7_01 | TNSGAAWNCGAAANTNNN | 1 | 1 | 1 | 6 | 2 | 1 | interferon regulatory factor 7 |
| IRF_Q6 | BNCRSTTTCANTTYY | 4 | 4 | 6 | 11 | 7 | 1 | Interferon regulatory factors |
| ISRE_01 | CAGTTTCWCTTTYCC | 2 | 1 | 2 | 4 | 1 | 1 | Interferon stimulated response element |
| LBP1_Q6 | CAGCTGS | 2 | 3 | 4 | 5 | 8 | 2 | TATA box repressor |
| LDSPOLYA_B | NNNSTGTGTDYYCWTN | 2 | 3 | 2 | 6 | 3 | 1 | Lentiviral Poly A downstream element |
| LFA1_Q6 | GGGSTCWR | 1 | 2 | 2 | 1 | 3 | 1 | AID1; HNF-2; LFA1 site |
| LMO2COM_01 | CNNCAGGTGBNN | 2 | 2 | 3 | 2 | 10 | 1 | LIM-only protein 2 site |
| MEIS1_01 | NNNTGACAGNNN | 1 | 2 | 2 | 5 | 5 | 2 | myeloid ecotropic viral integration site 1 |
| MYOD_Q6 | NNCACCTGNY | 2 | 3 | 2 | 7 | 6 | 1 | myoblast determining factor site |
| MYOGENIN_Q6 | RGCAGSTG | 2 | 4 | 4 | 8 | 7 | 1 | Myogenin site |
| NF1_Q6 | NNTTGGCNNNNNNCCNNN | 1 | 2 | 3 | 1 | 3 | 2 | nuclear factor 1 site |
| NFE2_01 | TGCTGAGTCAY | 3 | 1 | 1 | 3 | 1 | 1 | nuclear factor erythroid 2 p45 site |
| PIT1_Q6 | NMTTCATAWWTATNNMNA | 2 | 8 | 5 | 18 | 7 | 1 | Pit1, POU1F1 binding site |
| POU1F1_Q6 | ATGAATAAWT | 2 | 5 | 2 | 15 | 3 | 1 | POU1F1 binding site |
| PPAR_DR1_Q2 | TGACCTTTGNCCY | 1 | 2 | 5 | 1 | 3 | 1 | peroxisome proliferator-activated receptor |
| | | | | | | | | binding site |
| PU1_Q6 | WGAGGAAG | 5 | 5 | 4 | 2 | 6 | 2 | Pu.1 site, interfere with erythroblast |
| | | | | | | | | differentiation |
| SP1_01 | GGGGCGGGGT | 4 | 1 | 2 | 0 | 2 | 1 | Sp1 site, stimulating protein 1 |
| SP1_Q6 | NGGGGGCGGGGYN | 8 | 3 | 5 | 2 | 4 | 2 | Sp1 site, stimulating protein 1 |
| TAL1BETAE47_01 | NNNAACAGATGKTNNN | 1 | 2 | 1 | 3 | 2 | 1 | Tal-1beta/E47 heterodimer binding site |

TABLE S3
Gene-specific primers used for qRT-PCR assays.

| | | | | | | |
|---|---|---|---|---|---|---|
| | | | | Sequence | Accession | amplicon size |
| Gene name | Abbrevation | primer | primer sequences | ID No. | Number | (bp) |

| | | | | | | |
|---|---|---|---|---|---|---|
| Acetyl-CoA | Acc 1 | forward | AAGTCCTTGGTCGGGAAGTATACA | 43 | XM_109883 | 126 |
| carboxylase | | reverse | ACTCCCTCAAAGTCATCACAAACA | 44 | | |
| aP2 | Ap2 | forward | TTAAAAACACCGAGATTTCCTTCAA | 45 | NM_024406 | 102 |
| | | reverse | GGGCCCCGCCATCTAG | 46 | | |
| Carbohydrate | Chrebp | forward | CGGGACATGTTTGATGACTATGTC | 47 | AF156604 | 105 |
| regulatory element | | reverse | CATCCCATTGAAGGATTCAAATAAA | 48 | | |
| binding protein | | | | | | |
| Fasting-induced | Fiaf | forward | CAATGCCAAATTGCTCCAATT | 49 | AF278699 | 82 |
| adipose factor | | reverse | TGGCCGTGGGCTCAGT | 50 | | |
| Fatty acid | Fas | forward | TGGTGAATTGTCTCCGAAAAGA | 51 | AF127033 | 149 |
| synthase | | reverse | CACGTTCATCACGAGGTCATG | 52 | | |
| L32 ribosomal | L32 | forward | CCTCTGGTGAAGCCCAAGATC | 53 | NM_172086 | 102 |
| protein | | reverse | TCTGGGTTTCCGCCAGTTT | 54 | | |
| Peroxisome | Ppar-α | forward | CACCTTCCTCTTCCCAAAGCT | 55 | X57638 | 105 |
| proliferator | | reverse | GCGTCGGACTCGGTCTTCT | 56 | | |
| activated receptor | | | | | | |
| α | | | | | | |
| Peroxisome | Ppar-γ | forward | ATGTCTCACAATGCCATCAGGTT | 57 | U10374 | 116 |
| proliferator | | reverse | GCTCGCAGATCAGCAGACTCT | 58 | | |
| activated receptor | | | | | | |
| γ | | | | | | |
| Sterol regulatory | Srebp-1 | forward | GCATGCCATGGGCAAGTAC | 59 | NM_011480 | 125 |
| element binding | | reverse | CCACATAGATCTCTGCCAGTGTTG | 60 | | |
| protein 1 | | | | | | |

### EXAMPLE 13

Adult germ-free male NMRI/KI mice were maintained on a standard autoclaved chow diet rich in plant polysaccharides. Gas chromatographic-mass spectrometric (GC-MS) analysis established that glucose, arabinose, xylose and galactose are the predominant neutral sugars present in this chow (mole ratio=10:8:5:1). Seven week-old mice were colonized with a single inoculum of B. thetaiotaomicron and sacrificed 10 days later (a period that spans 2-3 cycles of turnover of the intestinal epithelium and its overlying mucus layer). Colonization density ranged from 10⁷-10⁹ CFU/mL in the distal small intestine (ileum) to 10¹⁰-10¹¹ CFU/mL in the cecum and proximal colon. Scanning electron microscopic studies revealed B. thetaiotaomicron attached to small food particles and embedded in mucus (FIG. 27).

The cecum is an anatomically distinct structure, located between the distal small intestine and proximal colon that is a site of great microbial density and diversity in conventionally-raised mice (F. Backhed, et al., Proc. Natl. Acad. Sci. USA, 101, 15718 (2004)). Nutrient use by B. thetaiotaomicron in the cecum was defined initially by whole genome transcriptional profiling. Cecal contents, including the mucus layer, were removed immediately after sacrifice of non-fasted mice (n=6), and the RNA extracted. The B. thetaiotaomicron transcriptome was characterized using custom GeneChips containing probe pairs derived from 4719 of the organism's 4779 predicted genes (Table S4). The results were compared to transcriptional profiles obtained from B. thetaiotaomicron grown from early log to stationary phase in a chemostat containing a minimal medium plus glucose as the sole fermentable carbohydrate source (MM-G; FIG. 30).

TABLE 54
Features of the B. theta GeneChip

| | Naming | No. of genes | No. of | Average no. of |
|---|---|---|---|---|
| | prefix of | (probesets) | probe | probe pairs |
| Functional category | probesets | represented | pairs | per probeset |
| Control sequences | AFFX | 51 | 831 | 16.3 |
| Bt chromosomal | BT | 4719 | 61737 | 13 |
| genes^{a} | | | | |
| Bt genes on p5482^{b} | p5482 | 38 | 494 | 13 |
| Bt tRNA genes | tRNA | 36 | 468 | 13 |

^{a}Genbenk accession number AE015928
^{b}Genbenk accession number AY171301

Unsupervised hierarchical clustering of the GeneChip datasets disclosed remarkable uniformity in the in vivo transcriptional profiles of B. thetaiotaomicron harvested from individual gnotobiotic mice (panel A, FIG. 31). A total of 1237 genes were defined as significantly upregulated in vivo compared with their expression in MM-G. The finctions of these upregulated genes were classified by clusters of orthologous groups (COG) analysis. The largest upregulated group belonged to the ‘carbohydrate transport and metabolism’ COG. In contrast, the largest group of genes down-regulated in vivo belonged to the ‘amino acid transport and metabolism’ COG (FIG. 32, A).

SusC and SusD are components of a B. thetaiotaomicron outer membrane protein complex involved in binding of starch and malto-oligosaccharides during their digestion by outer membrane and periplasmic glycoside hydrolases (J. A. Shipman, et al., J. Bacteriol. 182, 5365 (2000)). Thirty-seven SusC and 16 SusD paralogs are upregulated≧10-fold in vivo by comparison to bacteria growing in MM-G (FIG. 33).

The indigestibility of xylan, pectin, and arabinose-containing polysaccharides in dietary fiber reflects the paucity of host enzymes required for their degradation. The human genome contains only one putative glycoside hydrolase represented in the nine families of enzymes known in nature with xylanase, arabinosidase, pectinase, or pectate lyase activities, while the mouse genome has none (http://afmb.cnrs-mrs.fr/CAZY/). In contrast, B. thetaiotaomicron has 64 such enzymes (Table S5; http://afmb.cnrs-mrs.fr/CAZY/), many of which were selectively upregulated 10- to 823-fold in vivo. These included five secreted xylanases, five secreted arabinosidases, plus a secreted pectate lyase (FIG. 28A-C plus FIG. 33, B).

TABLE 55
All families of glycoside hydrolases and polysoccharide lyases containing arabinosidase,
xylanase, pectinase or pectate lyase activities with at least one representative in either
the human, mouse, or B. theta genomes (http://afmbenrsfrCAZY/).

| Family | Known Activities in Family | Homo sapiens | Mus musculus | B. theta |
|---|---|---|---|---|
| Glycoside Hydrolase Family 43 | β-xylosidase (EC 3.2.1.37) | 0 | 0 | 31 |
| | α-L-arabinofuranosidase (EC 3.2.1.55) | | | |
| | arabinanase (EC 3.2.1.99) | | | |
| | xylanase (EC 3.2.1.8) | | | |
| Glycoside Hydrolase Family 3 | β-glucosidase (EC 3.2.1.21) | 1 | 0 | 10 |
| | xylan 1,4-β-xylosidase (EC 3.2.1.37) | | | |
| | β-N-ocetylhexosaminidase (EC 3.2.1.52) | | | |
| | glucan 1,3-β-glucosidase (EC 3.2.1.58) | | | |
| | glucan 1,4-β-glucosidase (EC 3.2.1.74) | | | |
| | exo-1,3-1,4-glucanase (EC 3.2.1.-) | | | |
| | α-L-arabinofuranosidase (EC 3.2.1.55) | | | |
| Glycoside Hydrolase Family 28 | polygalacturonase (EC 3.2.1.15) | 0 | 0 | 9 |
| | exo-polygalacturonase (EC 3.2.1.67) | | | |
| | exo-polygalacturonase (EC 3.2.1.82) | | | |
| | rhamnogalacturonase (EC not defined) | | | |
| Polysaccharide Lyase Family 1 | pectate lyase (EC 4.2.2.2) | 0 | 0 | 5 |
| | pectin lyase (EC 4.2.2.10) | | | |
| Glycoside Hydrolase Family 51 | α-L-arabinofuranosidase (EC 3.2.1.55) | 0 | 0 | 4 |
| | endoglucanase (EC 3.2.1.4) | | | |
| Polysaccharide Lyase Family9 | pectate lyase (EC 4.2.2.2) | 0 | 0 | 2 |
| | exopolygalacturonate lyase (EC 4.2.2.9) | | | |
| Glycoside Hydrolase Family 5 | chitosanase (EC 3.2.1.132) | 0 | 0 | 1 |
| | β-mannosidase (EC 3.2.1.25) | | | |
| | cellulose (EC 3.2.1.4) | | | |
| | glucan 1,3-β-glucosidase (EC 3.2.1.58) | | | |
| | licheninase (EC 3.2.1.73) | | | |
| | glucan endo-1,6-β-glucosidase (EC 3.2.1.75) | | | |
| | mannan endo-1,4-β-manosidase (EC 3.2.1.78) | | | |
| | endo-1,4-β-xylanase (EC 3.2.1.8) | | | |
| | cellulose 1,4-β-cellobiosidase (EC 3.2.1.91) | | | |
| | endo-1,6-β-galactanase (EC 3.2.1.-) | | | |
| | β-1,3-mannanase (EC 3.2.1.-) | | | |
| Glycoside Hydrolase Family 93 | exo-arabinanase (EC 3.2.1.55) | 0 | 0 | 1 |
| Polysaccharide Lyase Family 10 | pectate lyase (EC 4.2.2.2) | 0 | 0 | 1 |

GC-MS analysis of total cecal contents harvested from fed germn-free mice revealed that xylose, galactose, arabinose, and glucose were the most abundant monosaccharide components (FIG. 28D). After 10 days of colonization by B. thetaiotaomicron, significant reductions in cecal concentrations of three prominent hexoses (glucose, galactose, and mannose) were observed. There were no significant decreases in pentose or amino-sugars (FIG. 28D). The selective depletion of hexoses likely reflects the combined effects of microbial and host utilization. B. thetaiotaomicron colonization increased host expression of the principal sodium/glucose transporter, Sglt1, in the intestinal epithelium, reflecting an enhancement of host utilization of liberated monosaccharides (Example 1 and Table 1). Morover, of the 1237 bacterial genes upregulated in vivo, 310 were assignable to enzyme classification numbers in metabolic maps in the Kyoto Encyclopedia of Genes and Genomes (KEGG; http://www.genome.adjp/). The results of this metabolic reconstruction were consistent with active delivery of mannose, galactose and glucose to the glycolytic pathway, and arabinose and xylose to the pentose phosphate pathway (FIG. 34; see http://gordonlab.wustl.edu/metaview/bt).

Host mucus provides a ‘consistent’ endogenous source of glycans in the cecal habitat that could offer alternative nutrients to the microbiota during periods of change in the host's diet. B. thetaiotaomicron embeds itself in this mucus layer (FIG. 27D). GeneChip analysis provided evidence that the bacterium harvests glycans from mucus. For example, in vivo, B. thetaiotaomicron exhibited significant upregulation (2-10-fold; p<0.05) of (i) an operon (BT0455-BT0461) that encodes a sialidase, sialic acid-specific 9-O-acetyl esterase, mannosidase, and three b-hexosaminidases (FIG. 28A), (ii) a mucin-desulfating sulfatase (BT3051), and (iii) a chondroitin lyase (BT3350). Fucose in host glycans is an attractive source of food: it typically occupies a terminal-linked position and is constitutively produced in the cecal mucosa of NMRI mice (L. Bry, et al., Science, 273, 1380 (1996)). In B. thetaioatomicron we found that two secreted a-fucosidases (BT1842, BT3665) and a five-component fucose utilization operon (BT1272-BT1277) were also induced (FIG. 28A). Operon induction, which occurs through the interaction of L-fucose with a repressor encoded by its first open reading frame (L. V. Hooper, et al., Proc. Natl. Acad. Sci. USA, 96, 9833 (1999)), is indicative of bacterial import and utilization of this hexose.

To determine whether the absence of fermentable polysaccharides in the diet increases foraging on mucus glycans, B. thetaiotaomicron gene expression was compared in the ceca of two groups of age- and gender-matched adult gnotobiotic mice. One group received the standard polysaccharide-rich chow diet from weaning to the time of sacrifice. The other group was switched to a diet devoid of fermentable polysaccharides but rich in simple sugars (35% glucose; 35% sucrose) 14 days prior to colonization. All mice were colonized with B. thetaiotaomicron for 10 days and bacterial gene expression was defined in each of their ceca at the time of sacrifice.

The presence or absence of polysaccharides in the diet did not produce a significant effect on the density of cecal colonization (data not shown). Using the transcriptional profiles of 98 B. thetaiotaomicron genes from the “replication, recombination and repair” COG as biomarkers, the cecal bacterial populations clustered most closely to cells undergoing log phase growth in vitro, irrespective of the diet (FIG. 31, B; Table S6).

TABLE 56
B. theta genes in the Replication Recombination and
Repair COG used for hierarchical clustering of
GeneChip data shown in panel B of FIG. 25

| Gene | Annotation |
|---|---|
| BT0026 | putative transposase |
| BT0069 | conserved hypothetical protein |
| BT0070 | conserved hypothetical protein |
| BT0078 | putative DNA repair protein |
| BT0244 | putative exonuclease |
| BT0245 | ATP-dependent exonuclease abcC |
| BT0252 | transcription-repair coupling factor |
| BT0280 | transposase for insertion sequence element 15RM3 |
| BT0358 | tranposase |
| BT0419 | putative endonuclease |
| BT0570 | excinuclease ABC subunit B |
| BT0578 | excinuclease ABC subunit A |
| BT0625 | DNA helicase |
| BT0630 | exodecoxyribonudease |
| BT0657 | ATP-dependent DNA helicase |
| BT0721 | DNA repair and recombination protein putative helicase |
| BT0831 | ATP-dependent RNA helicase |
| BT0894 | DNA ligase |
| BT0899 | DNA gyrase subunit A |
| BT1054 | ATP-dependent helicase |
| BT1081 | recombination protein recR |
| BT1154 | ATP-independent RNA helicase |
| BT1205 | putative ATPase AAA family |
| BT1756 | transposase invertase |
| BT1361 | DNA repair protein recN (Recombination protein N) |
| BT1364 | DNA polymerase III beta chain |
| BT1411 | methylated-DNA-protein-cysteine methyltransferase |
| BT1497 | single-strand binding protein (SSB) |
| BT1498 | AVG-specific adenine glycosylase |
| BT1499 | DNA-binding protein HU |
| BT1516 | replicative DNA helicase |
| BT1544 | NADH pyrophosphatase, Mutl family hydrolase |
| BT1610 | DNA polymerase III subunit gammaltau |
| BT1664 | crossover junction endodeoxyribonuclease ruvC |
| BT1671 | endonuclease III |
| BT1739 | excinuclease ABC subunit A |
| BT1821 | transposase |
| BT1848 | ATP-dependent DNA helicase recO |
| BT1885 | putative ATP-dependent RNA helicase |
| BT1978 | Holiday junction DNA helicase ruvA |
| BT1980 | transposase |
| BT2056 | conserved hypothetical protein |
| BT2073 | putative helicase |
| BT2089 | DNA topoisomerase II |
| BT2130 | uracil-DNA glycosylase |
| BT2137 | transposase |
| BT2143 | chromosomal replication initiator protein dnaA |
| BT2230 | DNA polymerase III alpha subunit |
| BT2297 | putative reverse transcriptase |
| BT2355 | site-specific DNA-methyltransferance |
| BT2400 | DNA-3-methyladenine glycosylase I |
| BT2615 | reverse transcriptase |
| BT2617 | reverse transcriptase |
| BT2644 | DNA topoisomerase I |
| BT2697 | DNA mismatch repair protein mut5 |

The simple sugar diet evoked a B. thetaiotaomicron tanscriptional response predominated by genes in the ‘carbohydrate transport and metabolism’ COG (FIG. 32, B). Glycoside hydrolase and polysaccharide lyase genes upregulated ≧2.5-fold in mice compated with MM-G cultures segregated into distinct groups after unsupervised hierarchical clustering (FIGS. 29). The group of 24 genes most highly expressed on the simple sugar diet encoded enzymes required for degradation of host glycans (e.g., eight hexosaminidases, two-fucosidases, plus a sialidase), and did not include any plant polysaccharide-directed arabinosidases or pectin lyases.

In addition, all components of the fucose utilization operon (BT1272-BT1277) were expressed at greater levels in mice fed the simple sugar diet compared to those fed the polysaccharide-rich diet (average induction compared to MM-G: 12-fold versus 6-fold). The sialylated glycan degradation operon (BT0455-BT0461) exhibited a comparable augmentation of expression on the simple sugar diet.

A similar cluster analysis revealed two distinct groups of genes encoding carbohydrate binding/importing SusC/SusD paralogs: a group of 61 expressed at highest levels in B. thetaiotaomicron from the ceca of mice fed a polysaccharide-rich diet, and a group of 21 expressed at highest levels with a simple sugar diet (FIG. 35). Thirteen of the upregulated SusC/D paralogs from B. thetaiotaomicron in mice fed a polysaccharide-rich diet are components of predicted operons that also contain ORFs specifying glycoside hydrolases and polysaccharide lyases. Five pairs of the SusC/D paralogs expressed at highest levels on a simple sugar diet are part of predicted operons. No SusC/D paralogs from one diet group were found in operons containing upregulated glycoside hydrolase genes from the other diet group (FIG. 36). Together, the data indicate that subsets of B. thetaiotaomicron's genome are dedicated to retrieving either host or dietary polysaccharides, depending upon their availability, although it appears that when both sources are available, harvesting energy from the diet is preferred.

Diet-associated changes in glycan foraging behavior were accompanied by changes in the expression of B. thetaiotaomicron's capsular polysaccharide synthesis (CPS) loci (FIG. 37). Compared with growth in MM-G, CPS3 was down-regulated in vivo irrespective of host diet, CPS4 was upregulated in the ceca of mice fed a polysaccharide-rich diet, while CPS5 was upregulated with a high sugar diet (FIG. 37). The other five CPS loci did not manifest significant differences in their expression during growth in vitro versus in vivo, or with diet manipulation. These findings suggest that B. thetaiotaomicron is able to change its surface carbohydrates depending upon the nutrient glycan environment that it is accessing and perhaps also for evasion of the host's adaptive immune response.

FIG. 38 presents a schematic overview of how B. thetaiotomicron might scavenge for carbohydrates in the distal intestine. Groups of bacteria assemble on undigested or partially digested food particles, elements of the mucus gel layer, and/or exfoliated epithelial cells. Bacterial attachment to these nutrient reservoirs is directed by glycan-specific outer membrane binding proteins (exemplified by SusC/D paralogs) that are opportunistically deployed depending upon the glycan environment encountered by the bacterium. Attachment helps oppose bacterial washout from the intestinal bioreactor, promotes harvest of oligo- and monosaccharides by an adaptively expressed repertoire of bacterial glycoside hydrolases, and facilitates sharing of the products of digestion with other microbial members whose nutritional niche overlaps that of B. thetaiotaomicron. In this scheme, microbial nutrient metabolism along the length of the intestine is a summation of myriad selfish and syntrophic relationships expressed by inhabitants of these micro-habitats. Micro-habitat diversity and mutualistic cooperation among component species (including the degree to which sanctions must be applied against cheats), are reflections of a dynamic interplay between the available nutrient foundation, and the degree of flexible foraging (niche breadth) expressed by micro-habitat residents. Members of Bacteroides with broad nutritional niches, such as B. thetaiotaomicron, contribute to diversity and stability by adaptively directing their glycan foraging behavior to the mucus when polysaccharide availability from the diet is reduced. Mucus glycans, in turn, represent a point where host genotype and diet intersect to regulate the stability of the microbiota. The highly variable outer chain structures of mucus and epithelial cell surface glycans are influenced by host genotype, and by microbial regulation of host glycosyltransferase gene expression. Co-evolution of glycan structural diversity in the host, and an elaborate repertoire of nutrient-regulated glycoside hydrolase genes in gut symbionts, endows the system with flexibility in adapting to changes in diet. While the present study has focused on the glycan foraging behavior of B. thetaiotaomicron in mono-associated germ-free mice, similar analyses can now be used to assess the impact of other members of the gut microbiota on B. thetaiotaomicron and on one another.

1. A method for treating obesity or an obesity-related disorder, the method comprising: (a) diagnosing a subject in need of treatment for obesity or an obesity-related disorder; and
(b) increasing either the amount of or the activity of a Fiaf polypeptide in the subject.

2. The method of claim 1, wherein the amount of Fiaf polypeptide is increased in the subject by administering an effective amount of Fiaf polypeptide to the subject.

3. The method of claim 2, wherein the subject is selected from the group consisting of a human, a dog, a cat, a cow, a horse, a rabbit, a pig, a sheep, a goat, an avian species and a fish species.

4. The method of claim 3, wherein the obesity related disorder is selected from the group consisting of metabolic syndrome, type II diabetes, hypertension, cardiovascular disease, and nonalcoholic fatty liver disease.

5. The method of claim 4, wherein the amount of or the activity of the Fiaf polypeptide is increased by administering a PPAR agonist to the subject.

6. A method for decreasing body fat or for promoting weight loss in a subject, the method comprising increasing either the amount of or the activity of a Fiaf polypeptide in the subject.

7. The method of claim 6, wherein the amount of Fiaf polypeptide is increased in the subject by administering an effective amount of a Fiaf polypeptide to the subject.

8. The method of claim 7, wherein the subject is selected from the group consisting of a human, a dog, a cat, a cow, a horse, a rabbit, a pig, a sheep, a goat, an avian species and a fish species.

9. The method of claim 8, wherein the amount of or the activity of the Fiaf polypeptide is increased by administering a PPAR agonist to the subject.

10. A method for decreasing body fat or for promoting weight loss in a subject, the method comprising altering the microbiota population in the subject's gastrointestinal tract such that at least one microbial-mediated signaling pathway in the subject that regulates energy storage is either stimulated or substantially inhibited, whereby stimulating or inhibiting the signaling pathway causes a decrease in body fat or promotes weight loss in the subject.

11. The method of claim 10, wherein the microbiota population is altered by decreasing the presence of at least one genera of saccharolytic microbe.

12. The method of claim 10, wherein the microbiota population is altered by decreasing the presence of B. thetaiotaomicron.

13. The method of claim 11, wherein the presence of a microbe genera is decreased by administering a probiotic selected from the group consisting of Lactobacillus, Acidophilus, Bifidobacteria and other components of the gut microbiota.

14. The method of claim 10, wherein the signaling pathway regulates hepatic lipogenesis and is substantially inhibited, thereby resulting in a decrease of triglyceride storage in the adipocytes of the subject.

15. The method of claim 14, wherein the amount of at least one compound selected from the group consisting of acetyl-CoA carboxylase, fatty acid synthase, sterol response element binding protein 1 and carbohydrate response element binding protein is decreased in the subject.

16. The method of claim 14, wherein hepatic lipogenesis is substantially inhibited as a result of a decrease in microbial processing of dietary polysaccharides.

17. The method of claim 14, wherein the signaling pathway substantially decreases lipoprotein lipase activity and results in a decrease of triglyceride storage in the adipocytes of the subject.

18. The method of claim 17, wherein lipoprotein lipase activity is substantially decreased as a result of microbial-mediated transcriptional suppression of a Fiaf polypeptide.

19. The method of claim 18, wherein microbial-mediated transcriptional suppression of the Fiaf polypeptide occurs only in the gastrointestinal tract of the subject.

20. The method of claim 10, wherein the subject is selected from the group consisting of a human, a dog, a cat, a cow, a horse, a rabbit, a pig, a sheep, a goat, an avian species and a fish species.

21. The method of claim 10, further comprising administering to the subject an effective amount of a Fiaf polypeptide.

22. A method for decreasing body fat or for promoting weight loss in a subject, the method comprising altering the microbiota population in the subject's gastrointestinal tract such that microbial-mediated transcriptional suppression of a lipoprotein lipase inhibitor in the subject is decreased.

23. The method of claim 22, wherein the lipoprotein lipase inhibitor is a Fiaf polypeptide.

24. The method of claim 23, wherein microbial-mediated transcriptional suppression of the Fiaf polypeptide occurs only in the gastrointestinal tract of the subject.

25. The method of claim 22, wherein the microbiota population is altered by decreasing the presence of at least one genera of saccharolytic microbe.

26. The method of claim 22, wherein the microbiota population is altered by decreasing the presence of B. thetaiotaomicron.

27. The method of claim 25, wherein the presence of a microbe is decreased by administering a probiotic selected from the group consisting of Lactobacillus, Acidophilus, Bifidobacteria and other components of the gut microbiota.

28. The method of claim 22, wherein the subject is selected from the group consisting of a human, a dog, a cat, a cow, a horse, a rabbit, a pig, a sheep, a goat, an avian species and a fish species.

29. The method of claim 22, further comprising administering to the subject an effective amount of a Fiaf polypeptide.

30. A method for treating obesity or an obesity-related disorder, the method comprising: (a) diagnosing a subject in need of treatment for obesity or an obesity-related disorder; and
(b) altering the microbiota population in the subject's gastrointestinal tract such that microbial-mediated transcriptional suppression of a lipoprotein lipase inhibitor in the subject is decreased.

31. The method of claim 30, wherein the lipoprotein lipase inhibitor is a Fiaf polypeptide.

32. The method of claim 31, wherein microbial-mediated transcriptional suppression of the Fiaf polypeptide occurs only in the gastrointestinal tract of the subject.

33. The method of claim 30, wherein the microbiota population is altered by decreasing the presence of at least one genera of saccharolytic microbe.

34. The method of claim 30, wherein the microbiota population is altered by decreasing the presence of B. thetaiotaomicron.

35. The method of claim 33, wherein the presence of a microbe genera is decreased by administering a probiotic selected from the group consisting of Lactobacillus, Acidophilus, Bifidobacteria and other components of the gut microbiota.

36. The method of claim 30, wherein the subject is selected from the group consisting of a human, a dog, a cat, a cow, a horse, a rabbit, a pig, a sheep, a goat, an avian species and a fish species.

37. The method of claim 30, wherein the obesity related disorder is selected from the group consisting of metabolic syndrome, type II diabetes, hypertension, cardiovascular disease, and nonalcoholic fatty liver disease.

38. The method of claim 30, further comprising administering to the subject an effective amount of a Fiaf polypeptide.

39. A composition for decreasing body fat or for promoting weight loss, the composition comprising a Fiaf polypeptide and an agent that alters the microbiota population in a subject's gastrointestinal tract such that microbial-mediated transcriptional suppression of a lipoprotein lipase inhibitor in the subject is decreased.

40. The composition of claim 39, wherein the agent is a probiotic selected from the group consisting of Lactobacillus, Acidophilus, Bifidobacteria and other components of the gut microbiota.

41. The composition of claim 39, wherein the composition further comprises a compound selected from the group consisting of acarbose, Xenical, orlistat, an amphetamine and sibutramine.

42. A biomarker for use in predicting whether a subject is at risk for becoming obese or suffering from an obesity-related condition, the biomarker comprising the amount of circulating Fiaf polypeptide.
